SEQUENCE LISTING (255 sequences)

SEQ ID NO: 1 - DNA, 65 bp, Artificial
  Synthetic DNA
gtagctggagaagggtgtgtctacctccagacatccttgaaatatagtgttctcccgagggagaa

SEQ ID NO: 2 - DNA, 65 bp, Artificial
  Synthetic DNA
tccagaacccctgactggtggctttggccttagcgatactttcattctgagtggatctgcttttg

SEQ ID NO: 3 - DNA, 65 bp, Artificial
  Synthetic DNA
tgaattacttcaagccactgacagaatggctcgtcaccgagaacaggagacatggagagacactg

SEQ ID NO: 4 - DNA, 65 bp, Artificial
  Synthetic DNA
ctctatggtacgaggtgtttagtatacccaagcagataggtgtcgatcgaacaggagcagggaga

SEQ ID NO: 5 - DNA, 65 bp, Artificial
  Synthetic DNA
ccccttacccccgtccttactttgtaacctggctgataacgggccatccatttttgtagcacact

SEQ ID NO: 6 - DNA, 65 bp, Artificial
  Synthetic DNA
tttctgggcagagtgaataacccccagagtgtggtgtgaggccttgtgcctagccatggagacaa

SEQ ID NO: 7 - DNA, 65 bp, Artificial
  Synthetic DNA
gctgaagagattaattagagaggtgtccagtggctccgaggagacgttcagctactctgactttc

SEQ ID NO: 8 - DNA, 65 bp, Artificial
  Synthetic DNA
cgtctgtactgtctacatcacggttcccttagcttgctcctggtagtgcattacaggcaagcatg

SEQ ID NO: 9 - DNA, 65 bp, Artificial
  Synthetic DNA
gcctgacctgatagagcagggtgattctgctggagctatgggtcaagatatggagacctggaagg

SEQ ID NO: 10 - DNA, 65 bp, Artificial; identical to SEQ ID NO: 9
  Synthetic DNA

SEQ ID NO: 11 - DNA, 65 bp, Artificial; identical to SEQ ID NO: 9
  Synthetic DNA

SEQ ID NO: 12 - DNA, 65 bp, Artificial
  Synthetic DNA
ccaagaggtcattgatctgggtggtgaggcaattaaaggtagtgagtactttggaaatggccgtg

SEQ ID NO: 13 - DNA, 65 bp, Artificial
  Synthetic DNA
attccttcacagaagtcattacactggactgatgacatgaggggccagaagcaaagccagccttg

SEQ ID NO: 14 - DNA, 65 bp, Artificial
  Synthetic DNA
gagttaagaggatccgatagcctatctctgataattctgtgtggaagcccccactgcaacactct

SEQ ID NO: 15 - DNA, 65 bp, Artificial
  Synthetic DNA
ggtgtgccctactcagtctccagaacagccctgtaggtccagccctctctgcaaccagagaacct

SEQ ID NO: 16 - DNA, 65 bp, Artificial
  Synthetic DNA
ctgcctgtcccatattataccaggtcacctaagaccttcctggattgatgctgacctatgaggta

SEQ ID NO: 17 - DNA, 65 bp, Artificial
  Synthetic DNA
ggaaatacgcactgagagaactgtaaaccacttagtcatgttactcgcgctggagaacgcactgg

SEQ ID NO: 18 - DNA, 65 bp, Artificial
  Synthetic DNA
gccgtttggaccagatgtcaaccaatatacaggctctaatgaaggagcatgcagagtcctatccc

SEQ ID NO: 19 - DNA, 65 bp, Artificial
  Synthetic DNA
ttatcaggagatggtggtagaggggtgtggatgccgctgagatcaggcagtccagagggcggaca

SEQ ID NO: 20 - DNA, 65 bp, Artificial
  Synthetic DNA
ctgtgcagtgcccggcttctattacttcaacttccaagtgatctccaagtgggacctttgtctgt

SEQ ID NO: 21 - DNA, 65 bp, Artificial
  Synthetic DNA
ccccttgactgcctgaaacccagaccagagccctgtagatgttacagaacgaatgggtcaataaa

SEQ ID NO: 22 - DNA, 65 bp, Artificial
  Synthetic DNA
cctgcggctccagaggggcgatgaggtgtggctatcagtcaatgactacaatggcatggtgggca

SEQ ID NO: 23 - DNA, 65 bp, Artificial
  Synthetic DNA
agttggcgcggttgcttcctgtagggtgaccctgccttggggaggagtgggttaggggttggata

SEQ ID NO: 24 - DNA, 65 bp, Artificial
  Synthetic DNA
gcatggaggcagggttgatcactgagccgtgttggttattcagttactattgctaacaacatggc

SEQ ID NO: 25 - DNA, 65 bp, Artificial
  Synthetic DNA
agttcaccagtactttaatgtgggacttatccagcccgggtcggtcaaggtctactcctattaca

SEQ ID NO: 26 - DNA, 65 bp, Artificial
  Synthetic DNA
ctttgagtccaagatcacccaagtcctgcatttcagaaaggacaccatggcctccataggtcaga

SEQ ID NO: 27 - DNA, 65 bp, Artificial
  Synthetic DNA
cccaggatggggagaagtgatacactggctttacttcggcacgtatttggaagcagtgactttag

SEQ ID NO: 28 - DNA, 65 bp, Artificial
  Synthetic DNA
aacaagaaggagatgaacatccaacagctcaccacctacaggaagagtgtcatgtccttggccga

SEQ ID NO: 29 - DNA, 65 bp, Artificial
  Synthetic DNA
ccaaaaccccatgagctaggcctcaactaccacaaaacagcctatgagctctgtttccaggattc

SEQ ID NO: 30 - DNA, 65 bp, Artificial
  Synthetic DNA
ccggtggaggctgacttcctgtatgcttactctacagcacctggttactattcctggagaaattc

SEQ ID NO: 31 - DNA, 65 bp, Artificial
  Synthetic DNA
aagaactgcgtttcctaccgagatcctgtgaatggaacctggtatattcagtcactttgccagag

SEQ ID NO: 32 - DNA, 65 bp, Artificial
  Synthetic DNA
tttgctttgtgaaagtccacttgagtttagatggtagatgtgccaagccttgtttgtttattttt

SEQ ID NO: 33 - DNA, 65 bp, Artificial
  Synthetic DNA
ggcctaaaatcacatgcctttcggagccgcagatctcttagcactgaaaaagtcccgtcatccct

SEQ ID NO: 34 - DNA, 65 bp, Artificial
  Synthetic DNA
atcaggaagttggtgagctggtataagacctcagtggagtgttccagggatgccatcgtgtttct

SEQ ID NO: 35 - DNA, 65 bp, Artificial
  Synthetic DNA
cacttctgtaggagtgaccagtgtgacagtgaactagtgtgactcggactgtgatgccttaatta

SEQ ID NO: 36 - DNA, 65 bp, Artificial
  Synthetic DNA
tctcctacagccggaagattccacgccaattcatcgttgactattttgaaaccagcagcctttgc

SEQ ID NO: 37 - DNA, 65 bp, Artificial
  Synthetic DNA
agcagtctttgctccaagccagctgtggtattcctgaccaaaagaggcagacagatctgtgctaa

SEQ ID NO: 38 - DNA, 65 bp, Artificial
  Synthetic DNA
cggtcctaagggataggagctgtctgtaggaatgtgaaacagtcacgcctaaggaatggtcttta

SEQ ID NO: 39 - DNA, 65 bp, Artificial
  Synthetic DNA
gctgctttcatgtactaaagctgaagatcccccttcgggtgctgaaaagctacgagagaatcaac

SEQ ID NO: 40 - DNA, 65 bp, Artificial
  Synthetic DNA
ttgagtccctcttctgtccggggctccaaccttctcagttgccaaaacgccccagtaccttccaa

SEQ ID NO: 41 - DNA, 65 bp, Artificial
  Synthetic DNA
ttgattgactaataataatgaggaaaacctgatgtgtacattacccgattgaaagtgtgtattgg

SEQ ID NO: 42 - DNA, 65 bp, Artificial
  Synthetic DNA
ggcgcatcagaaggggataaagaggactctgttttctcactagccactcacagatttctatctca

SEQ ID NO: 43 - DNA, 65 bp, Artificial
  Synthetic DNA
gtttaataaccttgacagttttgcatgtgatgctatcactcattggctacttgacatagccaacg

SEQ ID NO: 44 - DNA, 65 bp, Artificial
  Synthetic DNA
gagctggtgtgtgaatgttccagttgtgagctgtcagttaggggcagtgactgttcacttgctta

SEQ ID NO: 45 - DNA, 65 bp, Artificial
  Synthetic DNA
ctaccgctatctaaagggaacccccatttctgacccattagtagtcttgaatgtggggctctgag

SEQ ID NO: 46 - DNA, 65 bp, Artificial
  Synthetic DNA
tggccgatttaaacccaagttgcccagttctgagtagaaaactgagactatgctgtgtgtggcgg

SEQ ID NO: 47 - DNA, 65 bp, Artificial
  Synthetic DNA
gcctgggttacctatcacaactctgtctcagagaacagaaatactttccaggccagccagagctt

SEQ ID NO: 48 - DNA, 65 bp, Artificial
  Synthetic DNA
gtcgaatttgaagcaactgtgaattcacccagggaggactgtggagaccataggtgacaggtcat

SEQ ID NO: 49 - DNA, 65 bp, Artificial
  Synthetic DNA
cctgcagcgattcctgtcgactcccttacacagtattgatcttctgactgtggaaacaaccttcc

SEQ ID NO: 50 - DNA, 65 bp, Artificial
  Synthetic DNA
ggtgacagtgccacctctggcagctcccagaacactaaaaccacaaagtgtttaggttggacatt

SEQ ID NO: 51 - DNA, 65 bp, Artificial
  Synthetic DNA
aggtgtacatcaagaatggggacaagaaagccagttgtgagagagatgctacaaaggcccaaggc

SEQ ID NO: 52 - DNA, 65 bp, Artificial
  Synthetic DNA
ccgagactcaacagggaaatgtgggcctcctccacctattgacaatggagacatcacctccttgt

SEQ ID NO: 53 - DNA, 65 bp, Artificial
  Synthetic DNA
ccttcttatccatggggcacaactgcaataacttctgacctttgggcgaaagccgagaactcctg

SEQ ID NO: 54 - DNA, 65 bp, Artificial
  Synthetic DNA
tcccagcagcttctgtttacttttcttacacccatcaccggcatatcattgtactcggcaggggc

SEQ ID NO: 55 - DNA, 65 bp, Artificial
  Synthetic DNA
aagtccaagaactatgagttcatgtggaatcctcacctgggctacatcctcacatgcccatccaa

SEQ ID NO: 56 - DNA, 65 bp, Artificial
  Synthetic DNA
cagactacaggcacttttaagaacttgaccgaccttttcttctatgcgcagttggccacgacgtg

SEQ ID NO: 57 - DNA, 65 bp, Artificial
  Synthetic DNA
gggaagacctgaacagttatgacagtgatgaccaggaaaagcagtccaagaagaaaccccgcctc

SEQ ID NO: 58 - DNA, 65 bp, Artificial
  Synthetic DNA
ctcctgcaaacgcgatttgcccgtatcttggctgaatatgagtcgatgcagcagaaactcaagca

SEQ ID NO: 59 - DNA, 65 bp, Artificial
  Synthetic DNA
ccaaacgtgtagtccttcccatatttattgaggtgaaggtcctgctctgttacccagtccagcct

SEQ ID NO: 60 - DNA, 65 bp, Artificial
  Synthetic DNA
ctccgtgtcatttcttctcatcacatgggaatctcagcacatgagagccattatggggccaagca

SEQ ID NO: 61 - DNA, 65 bp, Artificial
  Synthetic DNA
cctgaagatgacgatgacttctctgagtactctgtgtattctgtggaggactaccaggagcctga

SEQ ID NO: 62 - DNA, 65 bp, Artificial
  Synthetic DNA
ttccccaggtcaaagccactaatccaatgggttctggcggtaagagattagtcaactcccagagg

SEQ ID NO: 63 - DNA, 65 bp, Artificial
  Synthetic DNA
ctagcctatcaggtctcccttccctggctacccagagtgaaactgattaaaaatgtggatcccac

SEQ ID NO: 64 - DNA, 65 bp, Artificial
  Synthetic DNA
attaacttcttggtcacatttctgagtaggctgaagtgcctgctttctaggtaggggtgagcgcc

SEQ ID NO: 65 - DNA, 65 bp, Artificial
  Synthetic DNA
tctacaaaagcggtgtctatgacgacccctcctgtacgggcaatgtgaatcatggtgttcttgtg

SEQ ID NO: 66 - DNA, 65 bp, Artificial
  Synthetic DNA
ctgctctgatggcaccgtctggtgaacgctggcttctgacaacactatacaatttcttttgaggg

SEQ ID NO: 67 - DNA, 65 bp, Artificial
  Synthetic DNA
ggagccagaccatcctggataatgtgagaacatgcctagatttacccacaaaacacaagtctgag

SEQ ID NO: 68 - DNA, 65 bp, Artificial
  Synthetic DNA
ggatttcaatgtaatgttgtgagtaacccttggacattttatgtcttcctcgtaaggcacagtgc

SEQ ID NO: 69 - DNA, 65 bp, Artificial
  Synthetic DNA
tgcgggaggacctagaggaacttcaggcccagtatcctaatcgctttaagctctggttcactctg

SEQ ID NO: 70 - DNA, 65 bp, Artificial
  Synthetic DNA
cctccctacaagacaggcttctcaaatggatgcccttgcaggcaagtcctagttttgttttgttt

SEQ ID NO: 71 - DNA, 65 bp, Artificial
  Synthetic DNA
gccctccaccaccgttagccaatccctgaacagtcccgtcttccaacccagagtcagtgacctcc

SEQ ID NO: 72 - DNA, 65 bp, Artificial
  Synthetic DNA
aggacacaattacctaagccacacctggagactgaacagtcatcctaatgactgacatcatggct

SEQ ID NO: 73 - DNA, 65 bp, Artificial
  Synthetic DNA
gaatccacgtgacacatgactctctttaggagtcacccacagttcttgtgtgtacagattgcttt

SEQ ID NO: 74 - DNA, 65 bp, Artificial
  Synthetic DNA
gtctacaaatacatgagcatccgatctgacaggtccgtgccttcagacatcttccagatacaggc

SEQ ID NO: 75 - DNA, 65 bp, Artificial
  Synthetic DNA
ttcttgacaccgcattgaagccactcacctgtgtgcgtctgggtatgaagtccagctccttccgt

SEQ ID NO: 76 - DNA, 65 bp, Artificial
  Synthetic DNA
aaccaggctgatgatggtagagtgctacagacttggtactccagtttccacggctaatcactgct

SEQ ID NO: 77 - DNA, 65 bp, Artificial
  Synthetic DNA
ccagaccccttcactccctgctctccgatttcagcacaataaatacactacagctgtgggaaaag

SEQ ID NO: 78 - DNA, 65 bp, Artificial
  Synthetic DNA
gttccgtaatccaaaggccaaatgaggagctggagactccaggctttcacaggaaagacacaggc

SEQ ID NO: 79 - DNA, 65 bp, Artificial
  Synthetic DNA
ggggcatggggcaacagtggaaacctgctgataccagtcatggtagtacttgtcattttcactga

SEQ ID NO: 80 - DNA, 65 bp, Artificial
  Synthetic DNA
agtggtataggtgcagatcaatggtgaagtgcttcttagcatgggacgagccctagattccatcc

SEQ ID NO: 81 - DNA, 65 bp, Artificial
  Synthetic DNA
acctatgcatcagctaaatggacacacagcggaggggaaatgttcgttgccttaaatcaaaaggg

SEQ ID NO: 82 - DNA, 65 bp, Artificial
  Synthetic DNA
tcaattctgccacctgctggttgctttggtaccttggtctcttattcaaacccacaccactcaag

SEQ ID NO: 83 - DNA, 65 bp, Artificial
  Synthetic DNA
ctctttggggtctagagatgagccctgggtctctaaaatggctctcttagaagttgtatgtgcaa

SEQ ID NO: 84 - DNA, 65 bp, Artificial
  Synthetic DNA
gtgacatgtccatctgcagtattgtgtgttttgatgccaacacggggacatcttcagttgttccc

SEQ ID NO: 85 - DNA, 65 bp, Artificial
  Synthetic DNA
gaaggagaagggctcactgcatacttatatccctgaaacgacatctttagagctggcctcatcac

SEQ ID NO: 86 - DNA, 65 bp, Artificial
  Synthetic DNA
cttcacttcagtgctgattcagcccagagggttagttagttccctctggacggctgctcttgtag

SEQ ID NO: 87 - DNA, 65 bp, Artificial
  Synthetic DNA
ggattcgcccattcctgtctcttcctacccaaccactcaattaatctttccttgcctgagaccag

SEQ ID NO: 88 - DNA, 65 bp, Artificial
  Synthetic DNA
cggtgaggtttctgtgacacctgacactgccagcctacttctctaatggattcctgtgtagctca

SEQ ID NO: 89 - DNA, 65 bp, Artificial
  Synthetic DNA
gggtggctcagagcagagtccaacccctattgatcaaccatgggcaaagaatatcccatgtgagg

SEQ ID NO: 90 - DNA, 65 bp, Artificial
  Synthetic DNA
gccagcgaccctaacgcccaggttccaaggctggttctgtttagtataagaacagaaaagcttca

SEQ ID NO: 91 - DNA, 65 bp, Artificial
  Synthetic DNA
cctcctcagccacgcccaagtgtactgggatacctaaggaccaatgttttcttcctcttagacac

SEQ ID NO: 92 - DNA, 65 bp, Artificial
  Synthetic DNA
ccaagtgaccctggtaagggaactgtctgcagaatggaagaaatagcctaagagacagggatggc

SEQ ID NO: 93 - DNA, 65 bp, Artificial
  Synthetic DNA
caaaaccccaggaaggttgtatgtgtcgggggacatacttctgatgaggtgctgagcattttgtc

SEQ ID NO: 94 - DNA, 65 bp, Artificial
  Synthetic DNA
gtgggatggctctcacctccagtctatgggaacttcaagcctttattgaaacacccaccagctga

SEQ ID NO: 95 - DNA, 65 bp, Artificial
  Synthetic DNA
gagtctctgcattggccagtaactatccttgtagctatgccccgtgtttgccaggcctgggaatc

SEQ ID NO: 96 - DNA, 65 bp, Artificial
  Synthetic DNA
tttcccatggtgcagagcaccggtctacatttactgccgagtgcgatccaatgctctggactgag

SEQ ID NO: 97 - DNA, 65 bp, Artificial
  Synthetic DNA
ggagtagagagagtccctggtgtcttagtttccacagtaaggtttgcctgtgtacagacctctgt

SEQ ID NO: 98 - DNA, 65 bp, Artificial
  Synthetic DNA
ggtcagtttcacagggttatgccattttattcaagtccgttgctccgttgtgcagtctccaccat

SEQ ID NO: 99 - DNA, 65 bp, Artificial
  Synthetic DNA
tggagcgactcatccatgagtgccgaggaattcacagataccgtgtttgccaagatcgacatcaa

SEQ ID NO: 100 - DNA, 65 bp, Artificial
  Synthetic DNA
tgcttcattccacagagtgatgtctgtggtctaaatttgcctggaggtgggggaaactttgccag

SEQ ID NO: 101 - DNA, 65 bp, Artificial
  Synthetic DNA
tcactggagctgtggtggcttttgtgatgaagatgagaaggagaaacacaggtggaaaaggaggg

SEQ ID NO: 102 - DNA, 65 bp, Artificial
  Synthetic DNA
caggacccactcaactatcctccatctgttatagagtgactcctctgtcaccatgccctgacttc

SEQ ID NO: 103 - DNA, 65 bp, Artificial
  Synthetic DNA
atctgttcccattagcaggtgaaggaagctagggctgaaacaagagttttccgcgctctcaggga

SEQ ID NO: 104 - DNA, 65 bp, Artificial
  Synthetic DNA
gtgcctgttgtgtttggaggtcaggagttgctgtgtatgacagtttcggctacgcttgactaaca

SEQ ID NO: 105 - DNA, 65 bp, Artificial
  Synthetic DNA
tatcagtgttccagtagcctgggaagacatatagtctagctgcccagttccctggagatggtcat

SEQ ID NO: 106 - DNA, 65 bp, Artificial
  Synthetic DNA
cgtatgtacatggagatttgcttgaaagtagaaccctgatgctcgcacacctgacctgtggaagc

SEQ ID NO: 107 - DNA, 65 bp, Artificial
  Synthetic DNA
ctacttttgttcccaatgtcagccaccatgccttagcagctgaacaatcgagcctcatgctcatg

SEQ ID NO: 108 - DNA, 65 bp, Artificial
  Synthetic DNA
ttgctgctttaggtgtctcttttcctccctctctatctctactctccaacatgaaggcgctgagc

SEQ ID NO: 109 - DNA, 65 bp, Artificial
  Synthetic DNA
ccaggaagatgccctgctggctgtgaggaaatacttccacaggatcactgtgtacctgagagaga

SEQ ID NO: 110 - DNA, 65 bp, Artificial
  Synthetic DNA
agctggctgcactccactagaattgccacgacgtacctatcacagtgacgtctacagtgagaaat

SEQ ID NO: 111 - DNA, 65 bp, Artificial
  Synthetic DNA
ggctccatccatatctcctatcggtgtctctgtatccttaaaccttgcaaacatcatacagtgta

SEQ ID NO: 112 - DNA, 65 bp, Artificial
  Synthetic DNA
tttctaagccagtgaggttgaggtgagaggtttgccagagtttgtctacctctgggtatcccttt

SEQ ID NO: 113 - DNA, 65 bp, Artificial
  Synthetic DNA
cagcatcggactccacgttcagagatgtcgcaaataatggtgcgcttagttcttcagatgacttc

SEQ ID NO: 114 - DNA, 65 bp, Artificial
  Synthetic DNA
ctgatccagggatcttagctaacggaaacaactccttggaaaacctcgtttgtacctctctccga

SEQ ID NO: 115 - DNA, 65 bp, Artificial
  Synthetic DNA
ttcctccagaatgtgaaggtcaacctcaaagtctttaactcccttggcgcaaaagtgagctccag

SEQ ID NO: 116 - DNA, 65 bp, Artificial
  Synthetic DNA
ggaacatccttaaatcctctgagcttgacaggcatcctcacagcaggattttctaggtggtcagt

SEQ ID NO: 117 - DNA, 65 bp, Artificial
  Synthetic DNA
cattaggcagcactctctagaacagaacctagctgtcaacgtgtgggggatgaattggtcatagc

SEQ ID NO: 118 - DNA, 65 bp, Artificial
  Synthetic DNA
atctactcggcaaacctagtgcgttatgcctaagcatatcagtttgtggacattcctcactgtgg

SEQ ID NO: 119 - DNA, 65 bp, Artificial
  Synthetic DNA
gtagataccaagtccccaggagctctaagtttcccaatgctcccattcactctccagctgtcttc

SEQ ID NO: 120 - DNA, 65 bp, Artificial
  Synthetic DNA
ggtgctatcgacggtgctacttcatctgtgtacaaaagaccgacgcatggtctatgttgctacca

SEQ ID NO: 121 - DNA, 65 bp, Artificial
  Synthetic DNA
ttgactacttggtcctcagtggaaccacttgggatgactagaggtgtggggttgttggttgagtt

SEQ ID NO: 122 - DNA, 65 bp, Artificial
  Synthetic DNA
ttgccgccttacgagaacagtgtgagtttctgtagtgagtctaaccccgaggagggaatttagtg

SEQ ID NO: 123 - DNA, 65 bp, Artificial
  Synthetic DNA
aaagccccatcttttggtatgccccggagtccctatctgacaacatcttctcccgccaatctgac

SEQ ID NO: 124 - DNA, 65 bp, Artificial
  Synthetic DNA
tggtctcactaccagttaaagcaaaagactttcaaacagtggctctgtcctccaagaagtggcaa

SEQ ID NO: 125 - DNA, 65 bp, Artificial
  Synthetic DNA
gccttttaaactttgtgtgttgtgtgtctgtgcacatgggtacaggtgcctgctcacttgagagg

SEQ ID NO: 126 - DNA, 65 bp, Artificial
  Synthetic DNA
tgacctccagcttcacccgagccaggagaaagtctttgtgaactgtgaagtgaagtcaaaaagac

SEQ ID NO: 127 - DNA, 65 bp, Artificial
  Synthetic DNA
actatgaggaggggcatctcaatggcacctttgaatcccaggttcagtaacgcccaccatctggc

SEQ ID NO: 128 - DNA, 65 bp, Artificial
  Synthetic DNA
gtgggaaaggtctaccttcccaaagagagaagacacatgaattcttggcagaggggatagcatgg

SEQ ID NO: 129 - DNA, 65 bp, Artificial
  Synthetic DNA
tatttctctgctgtttcagttcttgagaaattggggaagtcctacagaggctgcccctgccctca

SEQ ID NO: 130 - DNA, 65 bp, Artificial
  Synthetic DNA
tacgagacactgcagatggcgctcagctacatcatcgcgctcacccgcatcctagccgaagccga

SEQ ID NO: 131 - DNA, 65 bp, Artificial
  Synthetic DNA
ccgcctctgccatctgctacggtgtcgtcagttgtatggcattaatttcctgccactagagatat

SEQ ID NO: 132 - DNA, 65 bp, Artificial
  Synthetic DNA
gcttctccaagttgttccaagaaagggtgccctgtacctttccactggcaggacattaccattgt

SEQ ID NO: 133 - DNA, 65 bp, Artificial
  Synthetic DNA
cctagttggctgcctcccgccactctgactaaaaggaatcttaagagtgtacatttggaggtgga

SEQ ID NO: 134 - DNA, 65 bp, Artificial
  Synthetic DNA
cctcctctgtagttaaccagccttctccttcacctggtgacttcagatttaagagggtggcttct

SEQ ID NO: 135 - DNA, 65 bp, Artificial
  Synthetic DNA
actggagggctgtatctataaatctatttgccaataagttcccaggcagaggcaggtaggagggg

SEQ ID NO: 136 - DNA, 65 bp, Artificial
  Synthetic DNA
gggcgcggctccaaccgctgcataaatattaaggtattcagttgcccctactggaaggtattatg

SEQ ID NO: 137 - DNA, 65 bp, Artificial
  Synthetic DNA
agccaatgattttgggagtctgtagtatgaatggactagttattctgtgaaaacatctagagaaa

SEQ ID NO: 138 - DNA, 65 bp, Artificial
  Synthetic DNA
tgggccagcactcttagctttgataacttgacctgtggtatctctcgtggagaggtgtggctggc

SEQ ID NO: 139 - DNA, 65 bp, Artificial
  Synthetic DNA
ccgaatttggcgtccttcttggttctgaaatgaaatgtagttgccacgcacagacggtgtctagc

SEQ ID NO: 140 - DNA, 65 bp, Artificial
  Synthetic DNA
atcctgctgccctcttcatatgctttgatggactgtagactccctttaactctctctctggagta

SEQ ID NO: 141 - DNA, 65 bp, Artificial
  Synthetic DNA
ggacccaacatttaagggggaaatgtgggtgagaggaaagtggagtactctcagctaacacacag

SEQ ID NO: 142 - DNA, 65 bp, Artificial
  Synthetic DNA
ggggcagcacatcttagaagctaaatagttccctgcctttctcagccagtaattccacattcagg

SEQ ID NO: 143 - DNA, 65 bp, Artificial
  Synthetic DNA
gagttgggagtagccacctcatcagctactgtcatctgtcctctaaggggcataataaatgggag

SEQ ID NO: 144 - DNA, 65 bp, Artificial
  Synthetic DNA
gactcaaatcccacacaaccactgtaaggcatactcaggtcaagacatgagaagaccagcaggac

SEQ ID NO: 145 - DNA, 65 bp, Artificial
  Synthetic DNA
gtgaccaaatggaaagggttggtgtggaagcaagtgagacttcctaactgtacagcgtgggatgt

SEQ ID NO: 146 - DNA, 65 bp, Artificial
  Synthetic DNA
gcctccaacctgtcccatccatcaaagctttggccatgttttacctcccttctcatctatccttg

SEQ ID NO: 147 - DNA, 65 bp, Artificial
  Synthetic DNA
aaggagcccctcgaaacgaagaagtgcgtgttgagttatacgcaaggagtgctgggtactgatat

SEQ ID NO: 148 - DNA, 65 bp, Artificial
  Synthetic DNA
ctaacccctacagtggagcacaggactggatttattctggcagctactgggacagaaactacttc

SEQ ID NO: 149 - DNA, 65 bp, Artificial
  Synthetic DNA
acacaggctgttggatcgcggatctgtgttgctcatgtggttgtttaaaggaaaccatgatcgac

SEQ ID NO: 150 - DNA, 65 bp, Artificial
  Synthetic DNA
ccttcaggggtttcctcagctgcaggatattctgcatttttcagggggatctatgcctgagatgg

SEQ ID NO: 151 - DNA, 65 bp, Artificial
  Synthetic DNA
aagaagaaggaagaagacagagtcgcagccaagaaagtgggcacagaagtttgcaatggtggtcc

SEQ ID NO: 152 - DNA, 65 bp, Artificial
  Synthetic DNA
aacactgctgtccacatgacctccatttcccaaagtcctctgctccagcaactgcccttccaggt

SEQ ID NO: 153 - DNA, 65 bp, Artificial
  Synthetic DNA
gttgctgggacagagctgtagtgtcttggtcgtctgtgttggtacccaatacagtgtagacatct

SEQ ID NO: 154 - DNA, 65 bp, Artificial
  Synthetic DNA
tttattccccactaaagaaacggtttcctaaggtctgagctgtttcccagggtgggctagagtgg

SEQ ID NO: 155 - DNA, 65 bp, Artificial
  Synthetic DNA
taccggagccaaagtggagaggatgaatcccttaaccagcctggaccaatcaaaaccacttttgc

SEQ ID NO: 156 - DNA, 65 bp, Artificial
  Synthetic DNA
caccggttgtctctgccgggactaactgccaagccagattctcttgaataaagcattctagtctg

SEQ ID NO: 157 - DNA, 65 bp, Artificial
  Synthetic DNA
catgctggttaagatgcagcctagccattgcctgagctgttcatacaatgatccggaagtgctag

SEQ ID NO: 158 - DNA, 65 bp, Artificial
  Synthetic DNA
gcctggaaatgttctttcactgacttgtggcagcctgggaagtcatctgtgagcttctatgactt

SEQ ID NO: 159 - DNA, 65 bp, Artificial
  Synthetic DNA
aacttcacccttgtttagcccctattaaggtcgctttggatgtggggaaaggcccaacggtagaa

SEQ ID NO: 160 - DNA, 65 bp, Artificial
  Synthetic DNA
gatgaagagggctgagcgtaggtaatgcgggctctccccacatcctttctgaatgggcacttcta

SEQ ID NO: 161 - DNA, 65 bp, Artificial
  Synthetic DNA
cctctactctccgtttgcatgatccgctctgttagatgcattcattgtagttcggaagcaagcgt

SEQ ID NO: 162 - DNA, 65 bp, Artificial
  Synthetic DNA
gcgtgcactcagttccgtaggattccaaagcagacccctagctggtctttgaatctgcatgtact

SEQ ID NO: 163 - DNA, 65 bp, Artificial
  Synthetic DNA
aacaagggatcttgagagaaggaactgtcgctcagctgggagcggaatcattatcgcaatcacag

SEQ ID NO: 164 - DNA, 65 bp, Artificial
  Synthetic DNA
cctcaatctcacctctaccttcctcaggaaaaaccagtgtgagaccaagatcatggtactgcagc

SEQ ID NO: 165 - DNA, 65 bp, Artificial
  Synthetic DNA
ggcaagagagttctggagactgtgggacagctaggatgaagaactctatgttataagcctctgtc

SEQ ID NO: 166 - DNA, 65 bp, Artificial
  Synthetic DNA
tacaaagtccacatcaaccccaactcgctctttgacgtccaggtgaaaaggattcatgagtacaa

SEQ ID NO: 167 - DNA, 65 bp, Artificial
  Synthetic DNA
gaggaggaattcatcgaggggaccctggccaataaggaaattctgcgactgatccagtttgaacc

SEQ ID NO: 168 - DNA, 65 bp, Artificial
  Synthetic DNA
ttgtgtgtacctgtatggtacatgtgtgggagagccttcagagatgggaaggtgtggagagccgt

SEQ ID NO: 169 - DNA, 65 bp, Artificial
  Synthetic DNA
gcaagttctatacagagtttgatcggcataacaatcgcattggattcgccttggcccgctaaggc

SEQ ID NO: 170 - DNA, 65 bp, Artificial
  Synthetic DNA
cctgcccttgaggggattatatgagatttaagggacttatgtggccagcctacttcctggcatgc

SEQ ID NO: 171 - DNA, 65 bp, Artificial
  Synthetic DNA
cgcagggaaacagggaaccaatgcgctattctcattctaccgccactctgagcttaaggaactta

SEQ ID NO: 172 - DNA, 65 bp, Artificial
  Synthetic DNA
aattcgttactcagactcagggaaggaaaggtccctggggttataggagttaagagcaagcggag

SEQ ID NO: 173 - DNA, 65 bp, Artificial
  Synthetic DNA
agggcaaaagcctgcatatctcctggttctgaatgccaaagacttgagtgaaattgccagggctg

SEQ ID NO: 174 - DNA, 65 bp, Artificial
  Synthetic DNA
gggctttgacaccctcctcctcgctgcccgaaagacagtattctggctacctttggatcaagata

SEQ ID NO: 175 - DNA, 65 bp, Artificial
  Synthetic DNA
ggtggagttttgtacggctgttaaaggtggcccttctttgctatttaaggggctgaggtctttcc

SEQ ID NO: 176 - DNA, 65 bp, Artificial
  Synthetic DNA
ggctgatggatgatctggaccgtaacaaggatcaggaagtaaacttccaggagtatgtcgccttc

SEQ ID NO: 177 - DNA, 65 bp, Artificial
  Synthetic DNA
gccttgagggaagcccccggtagactctcaaagttatgctagatatcaaagcatgagcattcctc

SEQ ID NO: 178 - DNA, 65 bp, Artificial
  Synthetic DNA
caacccaagaaactcctgggctaagtatctgacagtctcacatctcaacagtgtgaattaagtgt

SEQ ID NO: 179 - DNA, 65 bp, Artificial
  Synthetic DNA
ggcccttccaaagctgcacaatgcccctcaattacggaggctgagaatgtagtgggtatacctct

SEQ ID NO: 180 - DNA, 65 bp, Artificial
  Synthetic DNA
atgaccgcttcctttacttcagcaactggctgcatggggacattcggcagtatgacatctctaac

SEQ ID NO: 181 - DNA, 65 bp, Artificial; identical to SEQ ID NO: 180
  Synthetic DNA

SEQ ID NO: 182 - DNA, 65 bp, Artificial
  Synthetic DNA
ttatagccaagatagccaaccccaaatgagactagaactccccaagtgttgacgcttcttcccgg

SEQ ID NO: 183 - DNA, 65 bp, Artificial
  Synthetic DNA
gaatcctggaccccagtagtacttaatgtctcagtgctctacagaacccccagagggaagctgat

SEQ ID NO: 184 - DNA, 65 bp, Artificial
  Synthetic DNA
ttcctcttcctgctctgggaccagcaacacaggttcccagtcttcatgggtcgtgtatatgaccc

SEQ ID NO: 185 - DNA, 65 bp, Artificial
  Synthetic DNA
tcacggccgtctaccgctgggacaagaagaataaggagatgaagtttgcagtcaaattcatgttc

SEQ ID NO: 186 - DNA, 65 bp, Artificial
  Synthetic DNA
acctgctggggcatgagtgtcacctgggcagagcttgctaggtcattaaacagagacatgatgac

SEQ ID NO: 187 - DNA, 65 bp, Artificial
  Synthetic DNA
ccttgtccgtcactcctttgaatgctgattttgtctcaagtcctttcagttcacatagccgtggc

SEQ ID NO: 188 - DNA, 65 bp, Artificial
  Synthetic DNA
ccactggctgaaagatggaaacgcaaacagtctgaccttctgaggactacaatcaagtaagagcc

SEQ ID NO: 189 - DNA, 65 bp, Artificial
  Synthetic DNA
cacttcctcttcagtccctaatcagcccccaaagaaacgacgtcataacaccacttcctcctcat

SEQ ID NO: 190 - DNA, 65 bp, Artificial
  Synthetic DNA
aggagactcctgagttaacactgggaagacattggccagtcctagtcatctctcggtcagtaggt

SEQ ID NO: 191 - DNA, 65 bp, Artificial
  Synthetic DNA
ctgctcagactatcacctgtacctccctgaataccagcgtttaaccttcaagacatcccatgtgc

SEQ ID NO: 192 - DNA, 65 bp, Artificial
  Synthetic DNA
cacctctccccaccccctgccacttgaaaccttctactaatcaagagaaacttccaagccaacgg

SEQ ID NO: 193 - DNA, 65 bp, Artificial
  Synthetic DNA
caacaacggaaagggcagccatgagtcaagtcagctggatgaaccaagtctggaaacacacagac

SEQ ID NO: 194 - DNA, 65 bp, Artificial
  Synthetic DNA
tcaccgttttgagggatgatgttttgtggcacgtgtgtgatcacagcctgatggttctggtcgtg

SEQ ID NO: 195 - DNA, 65 bp, Artificial
  Synthetic DNA
gaacttataaactgaaagggtatttaggaaggcaaggcttgggcatttttatggctttcaatcct

SEQ ID NO: 196 - DNA, 65 bp, Artificial
  Synthetic DNA
aggtttaggcaactaagttggagttttactcctaagctagaagcttcgcccagaccggtgtgctc

SEQ ID NO: 197 - DNA, 65 bp, Artificial
  Synthetic DNA
ctcatgcaggtgccttccgtctcaactgttccttggttaagagaaaagaactggctgggagacca

SEQ ID NO: 198 - DNA, 65 bp, Artificial
  Synthetic DNA
gtccaatgaccctgtgcttgtagagtggcgttttctttgcatccagagaggcagatttagacacc

SEQ ID NO: 199 - DNA, 65 bp, Artificial
  Synthetic DNA
agctacctgggtccattcctcccccaaccccagttccttctattttccaaaagataaaaaccaaa

SEQ ID NO: 200 - DNA, 65 bp, Artificial
  Synthetic DNA
aatcatatcttgcactgcctggaattaaggacaatccgttctttctgcaactgtcttttcacctc

SEQ ID NO: 201 - DNA, 65 bp, Artificial
  Synthetic DNA
gaatgctggtccactagtgggatttctagggttcaaaagtgacttcacttccgggtcatcatcag

SEQ ID NO: 202 - DNA, 65 bp, Artificial
  Synthetic DNA
gtgaagagtttctcatcacgggccgcctaaggaacgggaaatttcacatcaatgcctgcagcttc

SEQ ID NO: 203 - DNA, 65 bp, Artificial
  Synthetic DNA
cctccctcccttactcccgtcatgccagcaactcgcaatatttcagatgacgtttacatggtagc

SEQ ID NO: 204 - DNA, 65 bp, Artificial
  Synthetic DNA
tagatctaagtcagctgtttgggttgaggaggagagaacccgaggaaatgaccatgctctgggga

SEQ ID NO: 205 - DNA, 65 bp, Artificial
  Synthetic DNA
ggggaggaagaaaggtctaacatccttttccttcatcattctcatttctggacatgccttgtgag

SEQ ID NO: 206 - DNA, 65 bp, Artificial
  Synthetic DNA
aagctcttcgactccacgactcttgaacatcagaagactttccgaacagagcgtcctgtcaactc

SEQ ID NO: 207 - DNA, 65 bp, Artificial
  Synthetic DNA
ccttcagtgtctttgctagatcaagtgcagacgctgcacacaatctctagttcctctagttctgg

SEQ ID NO: 208 - DNA, 65 bp, Artificial
  Synthetic DNA
cgaatggctaaggaggtataacgccgatatttctaccttcccgttggaaaacgcacctattggac

SEQ ID NO: 209 - DNA, 65 bp, Artificial
  Synthetic DNA
gtttgcagaagtatgtagcacttgtcctccaagctttcaggtgtaagggggaaaggtgaaccaca

SEQ ID NO: 210 - DNA, 65 bp, Artificial
  Synthetic DNA
tgatcccttgctgaatgcaaggagctaaccagaaaagttctgcttgacaagtccccatcgttgaa

SEQ ID NO: 211 - DNA, 65 bp, Artificial
  Synthetic DNA
ctagcttgtcctgagaagatatttaattttgctaacactcagctctgccctcccttgtccccacc

SEQ ID NO: 212 - DNA, 65 bp, Artificial
  Synthetic DNA
aacaattgtcaaggaacctcatgtctcacctcaggggccagggtactctctcacttaaccaccct

SEQ ID NO: 213 - DNA, 65 bp, Artificial
  Synthetic DNA
ccaagtctgtgtttattgaaccatgtggattactgcgggagaggactggcactcatgtgcaaaaa

SEQ ID NO: 214 - DNA, 65 bp, Artificial
  Synthetic DNA
gacaaatgacttgtagcttcagatgtctttgcgccatcagcactcagaaaggaaggggtctgagg

SEQ ID NO: 215 - DNA, 65 bp, Artificial
  Synthetic DNA
cgctgtcttagtactgtcactgttgtaaaggcacatgttggaacacatccagtcctgctgacctg

SEQ ID NO: 216 - DNA, 65 bp, Artificial
  Synthetic DNA
gcgtctatttcttctctggagacaaatactaccgagtcaaccttagaacccggcgagtggactct

SEQ ID NO: 217 - DNA, 65 bp, Artificial
  Synthetic DNA
ccatcgtgcaccgcaagtgcttctaggcggactgttactgagctgcgttttacaccctttctttg

SEQ ID NO: 218 - DNA, 65 bp, Artificial
  Synthetic DNA
gggctgccatttgcagtggcaaagtggagattgttgccatcaacgaccccttcattgacctcaac

SEQ ID NO: 219 - DNA, 65 bp, Artificial
  Synthetic DNA
atcagatgaggatatgggattcggtctcttcgactaatcccgccaaagcaaccaagtcagcctgc

SEQ ID NO: 220 - DNA, 65 bp, Artificial
  Synthetic DNA
gcatgttgactcgtcctctgaaccaaagcacggacaggattaagagtgatccaactttcaagtcg

SEQ ID NO: 221 - DNA, 65 bp, Artificial
  Synthetic DNA
gcaggacctgtggccaagttcttagtagctgtttatctgtgtgtaggactgtagaactgtagagg

SEQ ID NO: 222 - DNA, 65 bp, Artificial
  Synthetic DNA
acttgattgatgaatggacctgagtttttacccggaggaccttagcacaagaagaactgttgtcc

SEQ ID NO: 223 - DNA, 65 bp, Artificial
  Synthetic DNA
gtccctgtgcccacagattgcacgtgtagggaggtgagtgcttgtatcccaaattggttctaggt

SEQ ID NO: 224 - DNA, 65 bp, Artificial
  Synthetic DNA
ccccaactttcaaacctctttaatttcctagctagaatcccacaagcagcatagacctgaaagtg

SEQ ID NO: 225 - DNA, 65 bp, Artificial
  Synthetic DNA
cctgctcttcgtgttctctgccctgctggagtacgccgccgtcaactttgtgtctcggcaacaca

SEQ ID NO: 226 - DNA, 65 bp, Artificial
  Synthetic DNA
ctgtcgctacatcactgaacctctgctccccacgggagccgtgactgtaatcgccctacgggtca

SEQ ID NO: 227 - DNA, 65 bp, Artificial
  Synthetic DNA
cccgtgcagagctgcgcttgcagagattaaaatcaagtgtggagcaacatgtggaactctaccag

SEQ ID NO: 228 - DNA, 65 bp, Artificial
  Synthetic DNA
aatcctctggaaccccacacaggtctctgttgaagactctgccaaccacgaggatcagtacgaaa

SEQ ID NO: 229 - DNA, 65 bp, Artificial
  Synthetic DNA
ttcaaggaccattctgctagatgccctactccctgtgggtgaaaagtgggcaaaggtctctaagg

SEQ ID NO: 230 - DNA, 65 bp, Artificial
  Synthetic DNA
gctggtacacacatgactcaaaacaacagatacatcattccctagcagtctggccagccccgttg

SEQ ID NO: 231 - DNA, 65 bp, Artificial
  Synthetic DNA
gtcaaggcagtaaggtgttcttgggagccacacttagactctttccaaagatgtggagggaacag

SEQ ID NO: 232 - DNA, 65 bp, Artificial
  Synthetic DNA
ggctgttggaatttacgcataaagcagactgcatagatccaatattgactgacccaagcatgtta

SEQ ID NO: 233 - DNA, 65 bp, Artificial
  Synthetic DNA
ctaggcttcttcaaatggagctccagaatgtgcacgggacacatccgatctatatttagagacct

SEQ ID NO: 234 - DNA, 65 bp, Artificial
  Synthetic DNA
tgaacgttgcagtgtgaagtctgtactggctagggatggtagcccagcgtgagttcaggcatgaa

SEQ ID NO: 235 - DNA, 65 bp, Artificial
  Synthetic DNA
aagcagcttccggctggtagatttttgtcttgatgggctgtccataagaactcccagttctttcc

SEQ ID NO: 236 - DNA, 65 bp, Artificial
  Synthetic DNA
aaataaagtggcagcccttagacactgtgacggtcgtgtgcattgtgggagtggacttttatgga

SEQ ID NO: 237 - DNA, 65 bp, Artificial
  Synthetic DNA
cctattcatcttgttggaaccccaccccattttccctgacagaacattcctaagtctcaagggct

SEQ ID NO: 238 - DNA, 65 bp, Artificial
  Synthetic DNA
ccctgtctgatttgtaggaatagataccctgcatgctatcattggctcatactctctcccccggc

SEQ ID NO: 239 - DNA, 65 bp, Artificial
  Synthetic DNA
aaacacgtcttccctcgaaggttcccgtcgacctagggaggaccttacctgttcgtgaaacacac

SEQ ID NO: 240 - DNA, 65 bp, Artificial
  Synthetic DNA
ttttgtgcctcaaagttcggttggggctaccttgggatcccaaacagctaatatggtcaccaact

SEQ ID NO: 241 - DNA, 65 bp, Artificial
  Synthetic DNA
gtggggggcagtttatttgttgagagtgtgaccaaaagttacatgtttgcacctttctagttgaa

SEQ ID NO: 242 - DNA, 65 bp, Artificial
  Synthetic DNA
taggcgcggagatgggggtgcccgaacgtcccaccctgctgcttttactctccttgctactgatt

SEQ ID NO: 243 - DNA, 65 bp, Artificial
  Synthetic DNA
gtagtgaatgaccaagaataaggagaaaagccaactccttcatcaggcatagagagctgcagcaa

SEQ ID NO: 244 - DNA, 65 bp, Artificial
  Synthetic DNA
cttgataaccgcagccacaaagccttagtgactttcctctacctggtaagacagagctcttcatg

SEQ ID NO: 245 - DNA, 65 bp, Artificial
  Synthetic DNA
cagaacacttagatggtgcagataaatcttgggaccacattcctctaagcacggtttctagagtg

SEQ ID NO: 246 - DNA, 65 bp, Artificial
  Synthetic DNA
gcctccgggaactttccaggttggcacctgaatgccttactctcagcagtctgaggctcgcttgc

SEQ ID NO: 247 - DNA, 65 bp, Artificial
  Synthetic DNA
atagtgcacaaggatggtgggaataaatgaagcatcccacactcaacctcccaacatccataact

SEQ ID NO: 248 - DNA, 65 bp, Artificial
  Synthetic DNA
gctgtggcattgatgatacctgcctcagtcaataaatactgaatatcaaagcagtggattgcgta

SEQ ID NO: 249 - DNA, 65 bp, Artificial
  Synthetic DNA
agcctagcaccccaaagtcatgcacccagtatcctcttgtatgactgtatatgtctatgtctggg

SEQ ID NO: 250 - DNA, 65 bp, Artificial
  Synthetic DNA
gcaagccagaataaagactgtgccagctggtcagtcgcccttttgagaccactcctttgtgctcc

SEQ ID NO: 251 - DNA, 65 bp, Artificial
  Synthetic DNA
ggacgacattgcaggcattcagaagttatatggaaagaggaacacgctgtgatagatgcagacag

SEQ ID NO: 252 - DNA, 65 bp, Artificial
  Synthetic DNA
ccagatggtactaggggacacagaagatctggaacagagccaagacgtcacatctgactgcagac

SEQ ID NO: 253 - DNA, 65 bp, Artificial
  Synthetic DNA
gtcctggcactgaagccagcatgagatccatcattcttatgtcagctcaagggtcaaaaggactt

SEQ ID NO: 254 - DNA, 65 bp, Artificial
  Synthetic DNA
cccgatgctgctttcccctgtggggatctcaggagctgtttgaggatatattttcactttgtgat

SEQ ID NO: 255 - DNA, 65 bp, Artificial
  Synthetic DNA
gtgtcggcataagccgaagatatcggtagagttatattgagcagatcccccggtgaaggatttaa

(19) United States
(12) Patent Application Publication
(10) Pub. No.: US 2016/0024582 A1
(43) Pub. Date: Jan. 28, 2016

(54) MICROARRAY FOR EVALUATING EYE DISEASE, AND EVALUATION METHOD OF EYE DISEASE

(71) Applicant: MITSUBISHI RAYON CO., LTD., Tokyo (JP)

(72) Inventors: Kenjiro IKUTA, Kanagawa (JP); Susumu ISHIDA, Hokkaido (JP); Atsuhiro KANDA, Hokkaido (JP)

(73) Assignee: MITSUBISHI RAYON CO., LTD., Tokyo (JP)

(21) Appl. No.: 14/774,989

(22) PCT Filed: Apr. 8, 2014
(86) PCT No.: PCT/JP14/60223

§ 371 Date: Sep. 11, 2015

(30) Foreign Application Priority Data
Aug. 4, 2013 (JP) 2013-080395

(51) Int. Cl. C12Q 1/68

(52) U.S. Cl. CPC C12Q 1/6883; C12Q 2600/158; C12Q 2600/112; G06F 19/24

(57) ABSTRACT

The purpose of the present invention is to provide a method for objectively evaluating the state of eye disease in a test organism. Provided is a microarray for evaluating the state of eye disease. Further, this method for evaluating the state of eye disease in a test organism is characterized by detecting, from a sample taken from the test organism, at least one gene from a prescribed gene group and comparing the obtained detection result with a control.

### FIELD OF THE INVENTION

The present invention relates to a method for evaluating conditions of an eye disease in a subject organism, and to a method for evaluating inhibitory or restorative functions on the eye disease of the subject organism by using the method for evaluating the conditions.

### BACKGROUND ART

Glaucoma, diabetic retinopathy, age-related macular degeneration, retinitis pigmentosa and the like are listed as the current leading causes of blindness. Those eye diseases are often caused by degenerated retinal cells. Therefore, to unravel the cause of an eye disease and establish its treatment, it is essential to examine what causes degeneration of retinal cells and how to unravel the mechanism.

However, much of the detail in how an eye disease develops is unknown, and there are few markers available that are effective in evaluating symptoms and developing therapeutic drugs, while not many methods are established for efficiently evaluating such markers. Conventionally employed diagnostic methods are ophthalmoscopy (fundus photography, radiography with contrast medium), measurement by optical coherence tomography, vision testing, Amsler testing and the like (non-patent publication 1). Also, genetic polymorphisms are compared to determine risk factors (patent publication 1). In addition, to develop therapeutic drugs, part of the gene is evaluated as a marker. However, for unraveling disease mechanisms and developing drugs, no method is available that enables efficient evaluation of molecular markers all at once. Thus, conventional methods such as Western Blotting and PCR have been employed, but such methods take a long time to conduct.

### PRIOR ART PUBLICATION

### Patent Publication

Patent publication 1: JP2007-528371A

### Non-Patent Publication

Non-patent publication 1: “Age-Related Macular Degeneration” [online], Japan Ophthalmological Society [Internet search conducted Jun. 6, 2012] (URL: http://www.nichigan.or.jp/public/disease/momaku_karei.jsp)

### SUMMARY OF THE INVENTION

### Problems to be Solved by the Invention

The present invention was carried out in consideration of the aforementioned problems. For evaluating conditions of eye diseases, the objective of the present invention is to provide a method that is capable of collectively analyzing molecular markers related to treatment or prevention of eye diseases such as age-related macular degeneration so that the genes essential to unraveling eye diseases are specified and efficient treatment and preventive methods are established.

### Solutions to the Problems

The inventors of the present invention have studied intensively to solve the above problems and have found that conditions of an eye disease in a subject organism are evaluated by focusing on how a particular gene is expressed. Accordingly, the present invention has been completed.

Namely, the present invention is described as follows.

(1) Equipped with probes to detect at least one type of gene selected from among gene clusters 1 below, a microarray for evaluating the condition of an eye disease.
(2) The microarray described in (1) above to evaluate the presence of a disorder in the periphery of a retina.
(3) The microarray described in (1) above to evaluate the presence of a disorder in the retina.
(4) The microarray described in (1) above to evaluate the presence of light damage to the retina.
(5) The microarray described in (1) above to evaluate the presence of inflammation in the retina.
(6) The microarray described in (1) above to evaluate the presence of a disorder in the retinal pigment epithelium or choroid.
(7) The microarray described in (1) above to evaluate the presence of light damage to the retinal pigment epithelium or choroid.
(8) The microarray described in (1) above to evaluate the presence of inflammation in the retinal pigment epithelium or choroid.
(9) Equipped with probes to detect at least one type of gene selected from among gene clusters below, a microarray to evaluate the presence of a disorder in the eye.
<gene clusters>
Hspa1b, Il1a, Gsk3a, H2-K1, IL1b
   (10) Equipped with probes to detect at least one type of gene selected from among gene clusters below, a microarray to evaluate the presence of a disorder in the periphery of a retina.
<gene clusters>
Hspa1b, Il1a, Gsk3a, H2-K1, IL1b
   (11) Equipped with probes to detect at least one type of gene selected from among gene clusters below, a microarray to evaluate the presence of a disorder in the retina.
<gene clusters>
Il1a, H2-K1, Il1b, Il6, Hspa2
   (12) Equipped with probes to detect at least one type of gene selected from among gene clusters below, a microarray to evaluate the presence of light damage to the retina.
<gene clusters>
Gsk3a, Rpe65, Gpr143, Hfe, Trip1
   (13) Equipped with probes to detect at least one type of gene selected from among gene clusters below, a microarray to evaluate the presence of inflammation in the retina.
<gene clusters>
H2-K1, Il6, Cxcl1, Hfe, Trip1
   (14) Equipped with probes to detect at least one type of gene selected from among gene clusters below, a microarray to evaluate the presence of a disorder in the retinal pigment epithelium or choroid.
<gene clusters>
Hspa1b, Il1a, Gsk3a, H2-K1, IL1b
   (15) Equipped with probes to detect at least one type of gene selected from among gene clusters below, a microarray to evaluate the presence of light damage to the retinal pigment epithelium or choroid.
<gene clusters>
Hspa1b, Gsk3a, Il1b, Icam1, Hspa2
   (16) Equipped with probes to detect at least one type of gene selected from among gene clusters below, a microarray to evaluate the presence of inflammation in the retinal pigment epithelium or choroid.
<gene clusters>
Hspa1b, Il1a, H2-K1, Il1b, Icam1
   (17) A microarray equipped with probes having at least one base sequence selected from among SEQ ID Nos. 1˜219 and 221˜254.
   (18) A method for evaluating the condition of an eye disease in a subject organism, characterized by using a control to compare the detection results of the gene obtained from a sample taken from the subject organism by using the microarray according to any of microarrays in (1)˜(17) above.
   (19) A method for evaluating inhibitory or restorative functions of a test substance for an eye disease, characterized by using a control to compare the detection results of the gene obtained from a sample taken from a subject organism that has contacted, ingested or been administered the test substance by using the microarray according to any of (1)˜(17) above.
   (20) A method for evaluating the condition of an eye disease in a subject organism, characterized by using a control to compare through a multivariate analysis the detection results of the gene obtained from a sample taken from the subject organism by using the microarray according to any of (1)˜(17) above.
   (21) A method for evaluating inhibitory or restorative functions of a test substance for an eye disease, characterized by using a control to compare through a multivariate analysis the detection results of the gene obtained from a sample taken from a subject organism that has contacted, ingested or been administered the test substance by using the microarray according to any of (1)˜(17) above.
<gene clusters 1>
A2m, Abca4, Ace, Adam17, Adam19, Adam28, Adam9, Adipor1, Adipor2, Agt, Aif1, Akt3, Amy2a2, Amy2a3, Amy2a4, Amy2a5, Angpt-1, Angpt-2, Aoc3, Apbb1, apln, Arr3, At1r, At2r, Atp6ap2, Best1, Bmp4, C1qa, C1qb, C1qc, Clql1, C1s, C3, C4, Calb1, Calb2, Casp14, Casp3, Casp8, Casp9, Cckbr, Ccl17, Ccl2, Ccl3, Ccl4, Ccl7, Ccl8, Ccnd3, Ccr2, Cd44, Cd45, Cd55, Cd59a, Cd74, Cdh1, Cdh3, Cdh5, Cdr2, Cebpd, Cfb, Cfh, Chga, Chrna7, Ckb, Cldn5, Clip1, Cnga1, Cnga3, Cntf, Col7a1, Col8a2, Cp, Cp, Crx, Ctgf, Ctnna1, Ctss, Cxcl1, Cxcl12, Cxcl2, cxcr4, Cyb5r1, Darc, Doc2b, Edn2, Ednrb, Efemp1, Efna5, Egf, egln3, Elovl4, Eno3, Epo, Erap1, esm1, Fgf16, Fgf7, Fgfr1, Flt1, Fos, Furin, Gabrb3, Gem, Gfap, Glut1, Gnao1, Gnat1, Gnat2, Gngt2, Gpnmb, Gpr143, Grem2, Grm2, Grm6, Gsk3a, Gsk3b, Guca1a, Guk1, H2-K1, Hfe, Hif1a, Hspa1a, Hspa1b, Hspa2, Icam1, Id3, Ifna1, Ifnr, Igf1, Igf1r, Igfbp3, Il10, Il17a, Il1a, Il1b, Il6, Il6r, Il6st, Irf6, Irs1, Isgf3g, Itgav, Jak3, Jun, Kdr, Lgals3, Lipc, Lmo1, Lox11, Mapk1, Mapk3, Mark2, Math5, Mef2c, Mkks, Mmp1, Mmp14, Mmp2, Mmp7, Mmp8, Mmp9, Msr1, Nes, Nfkb1, Nos3, Np, Nr2e3, Nr1, Nrp1, Nt5e, Opa1, Opn1mw, Opn1sw, Osbpl1a, Pax6, Pde6a, Pde6b, Pdgfb, Pdpn, Pecam1, Pex1, Pgf, Pig7, Pkia, Pla2g5, Polg2, Ppara, Prkca, Prnp, Prom1, Ptgds, Ptgs1, Ptgs2, Pxmp3, Pygm, Rcv1, Rdh9, Ren, Rho, Robo4, Rom1, Rpe65, Rs1, Rxrg, S100a6, Sag, Scd1, Sdc2, Sele, Selenbp1, Selenbp2, Se1p, Serpina3n, Serpinf1, Serping1, Sfrp5, Sil1, Slc16a1, Slc16a4, Slc1a3, Socs3, Sox9, Sparc, Spp1, Stat1, Stat3, Stat5a, Stat6, Synpr, Tgfb1, Tgfb2, Tgfb3, Tgfbr2, Timp1, Timp2, Timp3, Tlr3, Tlr4, Tnfa, Tnfrsf1a, Trip1, Ttpa, Tyrp1, Usp9x, Vcam1, Vegfa, Vegfb, Vegfc, Vegfd, Vldlr, Vtn
   (22) A method for evaluating the condition of an eye disease by using a microarray to measure the expression variation of a gene that is classified at least as (a)˜(c) or (d)˜(f) below respectively:
(a) a gene associated with both light damage and retinal inflammation caused by an inflammation-inducing substance in the retina;
(b) a gene associated with light damage to the retina, but excluding genes classified as (a) above;
(c) a gene associated with retinal inflammation caused by an inflammation-inducing substance, but excluding genes classified as (a) above;
(d) a gene associated with both light damage and inflammation caused by an inflammation-inducing substance in the retinal pigment epithelial cells and choroid;
(e) a gene associated with light damage to the retinal pigment epithelial cells and choroid, but excluding genes classified as (d) above;
(f) a gene associated with inflammation of the retinal pigment epithelial cells and choroid caused by an inflammation-inducing substance, but excluding genes classified as (d) above
   (23) A method for evaluating inhibitory or restorative functions of a test substance for an eye disease by using a microarray to measure the expression variation of a gene that is classified at least as (a)˜(c) or (d)˜(f) below respectively:
(a) a gene associated with both light damage to the retina and retinal inflammation caused by an inflammation-inducing substance;
(b) a gene associated with light damage to the retina, but excluding genes classified as (a) above;
(c) a gene associated with retinal inflammation caused by an inflammation-inducing substance, but excluding genes classified as (a) above;
(d) a gene associated with both light damage and inflammation caused by an inflammation-inducing substance in the retinal pigment epithelial cells and choroid;
(e) a gene associated with light damage to the retinal pigment epithelial cells and choroid, but excluding genes classified as (d) above;
(f) a gene associated with inflammation of the retinal pigment epithelial cells and choroid caused by an inflammation-inducing substance, but excluding genes classified as (d) above
   (24) The method described in (22) or (23) above, in which a gene classified as (a) is at least one cluster selected from a group of At1r, Jak3, Ccnd3, H2-K1, C1ql1, Id3, Tgfb2, Ckb, Gnat1, Efna5, Crx, Rom1, Arr3, Gsk3a, Adipor1, Hspa1b, Guk1, Abca4, egln3, Gngt2, Clip1, Prnp, Sparc, Elovl4, Gsk3b, Itgav, Vegfa, Vegfb, Vegfc, Vegfd, Sdc2 and Trip1;
a gene classified as (b) is at least one cluster selected from a group of At2r, Pig7, Pgf, Rxrg, Col7a1, Casp9, Pecam1, Rpe65, Cckbr, Cd59a, Opn1mw, Grm6, Pkia, Darc, Apbb1, Prom1, Adam9, Cyb5r1, Gpr143, Atp6ap2, Nr2e3, Pde6a, Nr1, Cnga1, Hif1a, Gnat2 and Mmp2;
a gene classified as (c) is at least one cluster selected from a group of Cxcl1, Il6, Selenbp2, Nfkb1, Cldn5, Sox9, Cp, Grm2, Pax6, Prkca, Mark2, Ppara, Gem, Opn1sw, Robo4, Rho, Glut1 and Pex1;
a gene classified as (d) is at least one cluster selected from a group of cxcr4, At2r, Vcam1, Mef2c, Pkia, Scd1, Loxl1, H2-K1, Pxmp3, Erap1, Pgf, Tgfb3, Pdpn, Gsk3a, Fgf7, Ccl2, Cntf, Col8a2, Pecam1, Cd59a, Rxrg, C1s, Ccl7, Osbpl1a, Glut1, Selenbp2, Serpinf1, Gpnmb, Hspa2, Nes, Stat1, Egf, C1qb, Mmp14, Timp2, Lmo1, Lgals3, Mmp8, Flt1, Cldn5, Mmp2, Vegfa, Vegfb, Vegfc, Vegfd, Hspa1b, Kdr and Stat6;
a gene classified as (e) is at least one cluster selected from a group of Cfb, Mmp9, Calb2, Robo4, Gpr143, Cd44, Pig7, Il1b, C3, Gem, Cd55, Cebpd, Stat3 and Cnd3; and a gene classified as (f) is at least one cluster selected from a group of Cxcl1, Timp1, Cxcl2, Il6, Igf1, Icam1, Lipc, At1r, Spp1, Ctss, C1qc, Nfkb1, Grem2, Abca4, Apbb1, Chrna7, Cyb5r1, Pdgfb, Isgf3g, Nos3 and Clip1.
   (25) The method described in any of (22)˜(24) above, characterized by using the determination formula below to evaluate the condition of an eye disease.

M=(Xli×η1/β1+ . . . +Xki×ηk/(βk)(/η1+ . . . + [symbol] k) determination formula

When M1 max+σₘ₁<M2 min is satisfied, the condition of an evaluation sample cluster is the same as that in each disease model.

[in the formula, “M” represents the Mahalanobis distance to show the distance from the base space, “Xni (n=1˜k)” represents a gene expression level or gene expression ratio, “ηn (n=1˜k)” represents a signal-to-noise ratio (S/N) in each sample, and “βn (n=1˜k) represents the sensitivity of each sample.

“M1” represents a normal sample cluster or a control sample cluster. “M1 max” indicates the maximum value of “M1” and “σ m1” represents the standard deviation from “M1”. “M2” represents an evaluation sample cluster, and “M2 min” indicates the minimum value of “M2”.

(26) A microarray to evaluate the condition of an eye disease in which the nucleic acids or part of the nucleic acids described in (i), (ii) or (iii) below are mounted.
(i) nucleic acids containing a gene selected from (a)˜(c) or (d)˜(f) below:
(a) a gene associated with both light damage to the retina and retinal inflammation caused by an inflammation-inducing substance;
(b) a gene associated with light damage to the retina, but excluding genes classified as (a) above;
(c) a gene associated with retinal inflammation caused by an inflammation-inducing substance, but excluding genes classified as (a) above;
(d) a gene associated with both light damage and inflammation caused by an inflammation-inducing substance in the retinal pigment epithelial cells and choroid;
(e) a gene associated with light damage to the retinal pigment epithelial cells and choroid, but excluding genes classified as (d) above;
(f) a gene associated with inflammation of the retinal pigment epithelial cells and choroid caused by an inflammation-inducing substance, but excluding genes classified as (d) above
   (ii) nucleic acids composed of a base sequence complementary to the base sequence of the nucleic acids in (i) above; and
   (iii) nucleic acids capable of hybridizing under stringent conditions with nucleic acids composed of a base sequence complementary to that in the nucleic acids in (i) or (ii) above.
   (27) A microarray described in (26) above, in which a gene classified as (a) is at least one cluster selected from a group of At1r, Jak3, Ccnd3, H2-K1, Clql1, Id3, Tgfb2, Ckb, Gnat1, Efna5, Crx, Rom1, Arr3, Gsk3a, Adipor1, Hspa1b, Guk1, Abca4, egln3, Gngt2, Clip1, Prnp, Sparc, Elovl4, Gsk3b, Itgav, Vegfa, Vegfb, Vegfc, Vegfd, Sdc2 and Trip1;
a gene classified as (b) is at least one cluster selected from a group of At2r, Pig7, Pgf, Rxrg, Col7a1, Casp9, Pecam1, Rpe65, Cckbr, Cd59a, Opn1mw, Grm6, Pkia, Darc, Apbb1, Prom1, Adam9, Cyb5r1, Gpr143, Atp6ap2, Nr2e3, Pde6a, Nr1, Cnga1, Hif1a, Gnat2 and Mmp2;
a gene classified as (c) is at least one cluster selected from a group of Cxcl1, Il6, Selenbp2, Nfkb1, Cldn5, Sox9, Cp, Grm2, Pax6, Prkca, Mark2, Ppara, Gem, Opn1sw, Robo4, Rho, Glut1 and Pex1;
a gene classified as (d) is at least one cluster selected from a group of cxcr4, At2r, Vcam1, Mef2c, Pkia, Scd1, Loxl1, H2-K1, Pxmp3, Erap1, Pgf, Tgfb3, Pdpn, Gsk3a, Fgf7, Ccl2, Cntf, Col8a2, Pecam1, Cd59a, Rxrg, C1s, Ccl7, Osbpl1a, Glut1, Selenbp2, Serpinf1, Gpnmb, Hspa2, Nes, Stat1, Egf, C1qb, Mmp14, Timp2, Lmo1, Lgals3, Mmp8, Flt1, Cldn5, Mmp2, Vegfa, Vegfb, Vegfc, Vegfd, Hspa1b, Kdr and Stat6;
a gene classified as (e) is at least one cluster selected from a group of Cfb, Mmp9, Calb2, Robo4, Gpr143, Cd44, Pig7, Il1b, C3, Gem, Cd55, Cebpd, Stat3 and Ccnd3; and
a gene classified as (f) is at least one cluster selected from a group of Cxcl1, Timp1, Cxcl2, Il6, Igf1, Icam1, Lipc, At1r, Spp1, Ctss, C1qc, Nfkb1, Grem2, Abca4, Apbb1, Chrna7, Cyb5r1, Pdgfb, Isgf3g, Nos3 and Clip1.
   (27) Equipped with a probe to detect at least one type of gene selected from among the clusters of Hspa1b, Gsk3a and H2-K1, a microarray to evaluate the condition of an eye disease.

### Effects of the Invention

The embodiments of the present invention are capable of conducting objective evaluation of the condition of an eye disease in a subject organism. Also, the methods according to the embodiments are capable of evaluating inhibitory or restorative functions of foods or drugs on eye diseases.

### BRIEF DESCRIPTION OF THE DRAWINGS

FIG. 1 is a view schematically showing an array fixing device to be used in the embodiments of the present invention;

FIG. 2A is a view showing measurement results of the gene expression variation in a retina;

FIG. 2B is a view showing measurement results of the gene expression variation in a retina;

FIG. 2C is a view showing measurement results of the gene expression variation in a retina;

FIG. 2D is a view showing measurement results of the gene expression variation in a retina;

FIG. 2E is a view showing measurement results of the gene expression variation in a retina;

FIG. 2F is a view showing measurement results of the gene expression variation in a retina;

FIG. 2G is a view showing measurement results of the gene expression variation in a retina;

FIG. 2H is a view showing measurement results of the gene expression variation in a retina;

FIG. 2I is a view showing measurement results of the gene expression variation in a retina;

FIG. 2J is a view showing measurement results of the gene expression variation in a retina;

FIG. 2K is a view showing measurement results of the gene expression variation in a retina;

FIG. 2L is a view showing measurement results of the gene expression variation in a retina;

FIG. 3A is a view showing measurement results of the gene expression variation in a retinal pigment epithelium and choroid;

FIG. 3B is a view showing measurement results of the gene expression variation in a retinal pigment epithelium and choroid;

FIG. 3C is a view showing measurement results of the gene expression variation in a retinal pigment epithelium and choroid;

FIG. 3D is a view showing measurement results of the gene expression variation in a retinal pigment epithelium and choroid;

FIG. 3E is a view showing measurement results of the gene expression variation in a retinal pigment epithelium and choroid;

FIG. 3F is a view showing measurement results of the gene expression variation in a retinal pigment epithelium and choroid;

FIG. 3G is a view showing measurement results of the gene expression variation in a retinal pigment epithelium and choroid;

FIG. 3H is a view showing measurement results of the gene expression variation in a retinal pigment epithelium and choroid;

FIG. 3I is a view showing measurement results of the gene expression variation in a retinal pigment epithelium and choroid;

FIG. 3J is a view showing measurement results of the gene expression variation in a retinal pigment epithelium and choroid;

FIG. 3K is a view showing measurement results of the gene expression variation in a retinal pigment epithelium and choroid;

FIG. 3L is a view showing measurement results of the gene expression variation in a retinal pigment epithelium and choroid; and

FIG. 3M is a view showing measurement results of the gene expression variation in a retinal pigment epithelium and choroid.

### DETAILED DESCRIPTION OF THE EMBODIMENTS

In the following, the present invention is described in detail. The embodiments below are examples to describe the present invention. The present invention is not limited to those embodiments. Thus, various modifications are possible within a scope that does not deviate from the gist of the present invention. Also, the present application is based upon and claims the benefit of priority to Japanese Patent Application No. 2013-080395, filed Apr. 8, 2013. The entire contents of the specification and accompanying drawings are incorporated herein by reference.

### 1. Summary

The present invention relates to a microarray used to evaluate the condition of an eye disease, and to a method for evaluating the condition of an eye disease in the subject organism by detecting a gene in a sample taken from a subject organism using the microarray. In addition, the present invention relates to a method for evaluating inhibitory or restorative functions of a test substance for an eye disease by retrieving a sample from a subject organism that has contacted, ingested or been administered the test substance, and by detecting the gene in the sample and measuring the gene expression variations.

The inventors of the present invention have conducted intensive studies about genes associated with many kinds of diseases and identified genes associated with eye diseases, especially age-related macular degeneration in the retina. The inventors have found that the conditions of eye diseases are evaluated by focusing on gene expression variations, and have completed the present invention.

Namely, the present invention relates to a method for evaluating the condition of an eye disease in a subject organism by using as an index the expression level of at least one type of gene selected from among predetermined gene clusters, and to a method for evaluating the functions (effects) of a test substance for the eye disease.

### 2. Gene

The gene to be detected in the present embodiment is at least one type of gene selected from among the gene clusters below. Each gene is denoted according to official symbols specified by NCBI (the National Center for Biotechnology Information). Each gene below is denoted by using an official symbol specified for the mouse species. However, when genes are common to other animal species, denoted symbols do not specify any particular animal species.
<gene clusters>
A2m, Abca4, Ace, Adam17, Adam19, Adam28, Adam9, Adipor1, Adipor2, Agt, Aif1, Akt3, Amy2a2, Amy2a3, Amy2a4, Amy2a5, Angpt-1, Angpt-2, Aoc3, Apbb1, apin, Arr3, At1r, At2r, Atp6ap2, Best1, Bmp4, C1qa, C1qb, C1qc, C1ql1, C1s, C3, C4, Calb1, Calb2, Casp14, Casp3, Casp8, Casp9, Cckbr, Ccl17, Ccl2, Ccl3, Ccl4, Ccl7, Ccl8, Ccnd3, Ccr2, Cd44, Cd45, Cd55, Cd59a, Cd74, Cdh1, Cdh3, Cdh5, Cdr2, Cebpd, Cfb, Cfh, Chga, Chrna7, Ckb, Cldn5, Clip1, Cnga1, Cnga3, Cntf, Col7a1, Col8a2, Cp, Cp, Crx, Ctgf, Ctnna1, Ctss, Cxcl1, Cxcl12, Cxcl2, cxcr4, Cyb5r1, Darc, Doc2b, Edn2, Ednrb, Efemp1, Efna5, Egf, egln3, Elovl4, Eno3, Epo, Erap1, esm1, Fgf16, Fgf7, Fgfr1, Flt1, Fos, Furin, Gabrb3, Gem, Gfap, Glut1, Gnao1, Gnat1, Gnat2, Gngt2, Gpnmb, Gpr143, Grem2, Grm2, Grm6, Gsk3a, Gsk3b, Guca1a, Guk1, H2-K1, Hfe, Hif1a, Hspa1a, Hspa1b, Hspa2, Icam1, Id3, Ifna1, Ifnr, Igf1, Igf1r, Igfbp3, Il10, Il17a, Il11a, Il1b, Il6, Il6r, Il6st, Irf6, Irs1, Isgf3g, Itgav, Jak3, Jun, Kdr, Lgals3, Lipc, Lmo1, Loxl1, Mapk1, Mapk3, Mark2, Math5, Mef2c, Mkks, Mmp1, Mmp14, Mmp2, Mmp7, Mmp8, Mmp9, Msr1, Nes, Nfkb1, Nos3, Np, Nr2e3, Nr1, Nrp1, Nt5e, Opa1, Opn1mw, Opn1sw, Osbpl1a, Pax6, Pde6a, Pde6b, Pdgfb, Pdpn, Pecam1, Pex1, Pgf, Pig7, Pkia, Pla2g5, Polg2, Ppara, Prkca, Prnp, Prom1, Ptgds, Ptgs1, Ptgs2, Pxmp3, Pygm, Rcv1, Rdh9, Ren, Rho, Robo4, Rom1, Rpe65, Rs1, Rxrg, S100a6, Sag, Scd1, Sdc2, Sele, Selenbp1, Selenbp2, Se1p, Serpina3n, Serpinf1, Serping1, Sfrp5, Sil1, Slc16a1, Slc16a4, Slc1a3, Socs3, Sox9, Sparc, Spp1, Stat1, Stat3, Stat5a, Stat6, Synpr, Tgfb1, Tgfb2, Tgfb3, Tgfbr2, Timp1, Timp2, Timp3, Tlr3, Tlr4, Tnfa, Tnfrsf1a, Trip1, Ttpa, Tyrp1, Usp9x, Vcam1, Vegfa, Vegfb, Vegfc, Vegfd, Vldlr, Vtn

The expression levels of a gene included in the above gene clusters vary depending on the condition of an eye disease in a subject organism. The genes to be detected in the present embodiment are classified as shown in Table 4.
(i) genes that show variation in the expression level when light damage occurs in the retina
(ii) genes that show variation in the expression level when inflammation is caused in the retina
(iii) genes that show variation in the expression level when light damage occurs in the retinal pigment epithelium or choroid
(iv) genes that show variation in the expression level when inflammation is caused in the retinal pigment epithelium or choroid

According to the embodiments of the present invention, by detecting at least one type of gene selected from among gene clusters described in (i)˜(iv) above respectively and by comparing the detection results with a control, the condition of an eye disease in a subject organism is evaluated. For example, by detecting at least one type of gene selected from among gene clusters in (i) and by comparing the detected results with a control, whether or not light damage has occurred in the retina of the subject organism is objectively evaluated. Also, the method is capable of evaluating the condition of retinal disorder such as light damage to the retina or age-related macular degeneration caused by light damage. The same applies to other gene clusters.

In addition, genes subject to being detected in another embodiment of the present invention are classified as (a)˜(c) or (d)˜(f) below.
(a) a gene associated with both light damage to the retina and retinal inflammation caused by an inflammation-inducing substance;
(b) a gene associated with light damage to the retina, but excluding genes classified as (a) above;
(c) a gene associated with retinal inflammation caused by an inflammation-inducing substance, but excluding genes classified as (a) above;
(d) regarding retinal pigment epithelial cells and choroid, a gene associated with both light damage and inflammation caused by an inflammation-inducing substance; (e) a gene associated with light damage to the retinal pigment epithelial cells and choroid, but excluding genes classified as (d) above;
(f) a gene associated with inflammation of the retinal pigment epithelial cells and choroid caused by an inflammation-inducing substance, but excluding genes classified as (d) above

A gene classified as (a) above is at least one type selected from among At1r, Jak3, Ccnd3, H2-K1, Clql1, Id3, Tgfb2, Ckb, Gnat1, Efna5, Crx, Rom1, Arr3, Gsk3a, Adipor1, Hspa1b, Guk1, Abca4, egln3, Gngt2, Clip1, Prnp, Sparc, Elovl4, Gsk3b, Itgav, Vegfa, Vegfb, Vegfc, Vegfd, Sdc2 and Trip1; preferably at least one type selected from among At1r, Jak3, Ccnd3, H2-K1, C1ql1, Id3, Tgfb2, Ckb, Gnat1, Efna5, Crx, Rom1, Arr3, Gsk3a, Adipor1, Hspa1b, Guk1, Abca4, egln3, Vegfa, Vegfb, Vegfc and Vegfd; more preferably at least one type selected from among At1r, Jak3, Ccnd3, H2-K1, C1ql1, Id3, Tgfb2, Vegfa, Vegfb, Vegfc and Vegfd;
a gene classified as (b) above is at least one type selected from among At2r, Pig7, Pgf, Rxrg, Col7a1, Casp9, Pecam1, Rpe65, Cckbr, Cd59a, Opn1mw, Grm6, Pkia, Darc, Apbb1, Prom1, Adam9, Cyb5r1, Gpr143, Atp6ap2, Nr2e3, Pde6a, Nr1, Cnga1, Hif1a, Gnat2 and Mmp2; preferably at least one type selected from among At2r, Pig7, Pgf, Rxrg, Col7a1, Casp9, Pecam1, Rpe65, Cckbr, Cd59a, Opn1mw, Grm6, Pkia, Darc, Apbb1, Prom1, Adam9, Cyb5r1, Gpr143 and Atp6ap2; more preferably at least one type selected from among At2r, Pig7, Pgf, Rxrg, Col7a1 and Casp9;
a gene classified as (c) above is at least one type selected from among Cxcl1, Il6, Selenbp2, Nfkb1, Cldn5, Sox9, Cp, Grm2, Pax6, Prkca, Mark2, Ppara, Gem, Opn1sw, Robo4, Rho, Glut1 and Pex1; preferably at least one type selected from among Cxcl1, Il6, Selenbp2, Nfkb1, Cldn5, Sox9, Cp, Grm2, Pax6, Prkca, Mark2, Ppara and Gem; more preferably at least one type selected from among Cxcl1, Il6, Selenbp2 and Nfkb1;
a gene classified as (d) above is at least one type selected from among cxcr4, At2r, Vcam1, Mef2c, Pkia, Scd1, Loxl1, H2-K1, Pxmp3, Erap1, Pgf, Tgfb3, Pdpn, Gsk3a, Fgf7, Ccl2, Cntf, Col8a2, Pecam1, Cd59a, Rxrg, C1s, Ccl7, Osbpl1a, Glut1, Selenbp2, Serpinf1, Gpnmb, Hspa2, Nes, Stat1, Egf, C1qb, Mmp14, Timp2, Lmo1, Lgals3, Mmp8, Flt1, Cldn5, Mmp2, Vegfa, Vegfb, Vegfc, Vegfd, Hspa1b, Kdr and Stat6; preferably at least one type selected from among cxcr4, At2r, Vcam1, Mef2c, Pkia, Scd1, Loxl1, H2-K1, Pxmp3, Erap1, Pgf, Tgfb3, Pdpn, Gsk3a, Fgf7, Ccl2, Cntf, Col8a2, Pecam1, Cd59a, Rxrg, C1s, Ccl7, Osbpl1a, Glut1, Selenbp2, Serpinf1, Gpnmb, Hspa2, Vegfa, Vegfb, Vegfc and Vegfd; more preferably at least one type selected from among cxcr4, At2r, Vcam1, Mef2c, Pkia, Scd1, Loxl1, H2-K1, Pxmp3, Erap1, Pgf, Tgfb3, Pdpn, Gsk3a, Fgf7, Vegfa, Vegfb, Vegfc and Vegfd;
a gene classified as (e) above is at least one type selected from among Cfb, Mmp9, Calb2, Robo4, Gpr143, Cd44, Pig7, Il1b, C3, Gem, Cd55, Cebpd, Stat3 and Ccnd3; preferably at least one type selected from among Cfb, Mmp9, Calb2, Robo4, Gpr143, and Cd44; more preferably at least one type selected from among Cfb, Mmp9, Calb2, and Robo4; and
a gene classified as (f) above is at least one type selected from among Cxcl1, Timp1, Cxcl2, Il6, Igf1, Icam1, Lipc, At1r, Spp1, Ctss, C1qc, Nfkb1, Grem2, Abca4, Apbb1, Chrna7, Cyb5r1, Pdgfb, Isgf3g, Nos3, and Clip1; preferably at least one type selected from among Cxcl1, Timp1, Cxcl2, Il6, Igf1, Icam1, Lipc, At1r, Spp1, Ctss, C1qc, and Nfkb1; and more preferably at least one type selected from among Cxcl1, Timp1, Cxcl2, Il6, and Igf1.

A gene classified as any of (a)˜(c) above and a gene classified as any of (d)˜(f) above may be combined freely to be used for evaluating the condition of an eye disease. More specifically, the following combinations may be used:
a combination of a gene classified as (a) and a gene classified as (d);
a combination of a gene classified as (a) and a gene classified as (e);
a combination of a gene classified as (a) and a gene classified as (f);
a combination of a gene classified as (b) and a gene classified as (d);
a combination of a gene classified as (b) and a gene classified as (e);
a combination of a gene classified as (b) and a gene classified as (f);
a combination of a gene classified as (c) and a gene classified as (d);
a combination of a gene classified as (c) and a gene classified as (e); and a combination of a gene classified as (c) and a gene classified as (f).

Also, to distinguish light damage to the retina from retinal inflammation caused by an inflammation-inducing substance, a combination of Cxcl1, At2r, Vegfa, Cxcl1, Cfb and Fgf7 may be used for purposes of making such distinction. Regarding disorder in the retinal pigment epithelium and choroid, to distinguish light damage from inflammation caused by an inflammation-inducing substance, a combination of Il6, Pig7, Vegfa, Timp1, Mmp9 and Vegfc may be used for such purposes of distinction.

In yet another embodiment, it is preferred to detect at least one type of gene selected from among Hspa1b, Gsk3a and H2-K1 when evaluating the condition of an eye disease.

In the embodiments of the present invention, an “associated gene” indicates such a gene that shows variation in its expression level in the case of light damage or inflammation caused by an inflammation-inducing substance in the retina, retinal pigment epithelial cells and/or choroid. In the present invention, the condition of an eye disease is evaluated by detecting the associated gene (by determining its expression level). Also, in the embodiments of the present invention, “nucleic acids” include RNA and DNA (cDNA or the like). Moreover, in the embodiments of the present invention, “part of nucleic acids” indicates a polynucleotide having part of the base sequence of nucleic acids. Such nucleic acids may be used as later-described probes.

In the embodiments of the present invention, “at least one type selected from among” gene clusters is defined to include “all the genes” of gene clusters and “combinations of all the genes” of gene clusters.

### 3. Probe

In the embodiments of the present invention, “to detect a gene” means to determine the expression level of the gene, that is, to measure the level of mRNA or the level of nucleic acids amplified from DNA or mRNA. A probe to detect a gene means the nucleic acids to be hybridized under stringent conditions with its mRNA, cDNA or their antisense strand.

Stringent conditions for hybridization are, for example, “0.12M Tris-HCl/0.12M NaCl/0.5% Tween-20, 50° C.”, “0.12M Tris.HCl/0.12M NaCl/0.5% Tween-20, 42° C.” or “0.12M Tris.HCl/0.12M NaCl/0.5% Tween-20, 37° C.”; more stringent conditions are, for example, “0.12M Tris.HCl/0.12M NaCl/0.5% Tween-20, 65° C.,” “0.12M Tris-HCl/0.12M NaCl/0.5% Tween-20, 68° C.” or “0.06M Tris-HCl/0.06M NaCl/0.5% Tween-20, 65° C.”. More specifically, the temperature is maintained at 65° C. for at least an hour after a probe is added so as to hybridize the nucleic acids. Then, washing is conducted at 65° C. for 20 minutes 2˜4 times in a buffer of 0.12M Tris-HCl/0.12M NaCl/0.5% Tween-20, and a final washing is conducted at 65° C. for 10 minutes in a buffer of 0.12M Tris-HCl/0.12M NaCl. More stringent conditions may be set by increasing the temperature for hybridization or washing. In addition to the conditions of a buffer such as base concentration and temperature, it is an option for a person in the art to add several more conditions such as probe concentration, probe length and reaction time. “Molecular Cloning, A Laboratory Manual, 2nd ed.” (Cold Spring Harbor Press, 1989), “Current Protocols in Molecular Biology” (John Wiley & Sons, 1987-1997) or the like may be referred to for detailed procedures of hybridization methods.

Also, the embodiments of the present invention provide probes having base sequences shown in SEQ ID Nos. 1˜219 and 221˜254. Probes having base sequences shown in SEQ ID Nos. 1˜219 and 221˜254 (probe numbers 1˜219 and 221˜254) are shown in Table 1 below. In addition, as for a microarray, it is sufficient if it is mounted with probes having base sequences listed in SEQ ID Nos. 1˜219 and 221˜254. The microarray may further mount negative control (N.C.) probes. As for N.C. probes, they are not limited specifically as long as no gene of the target animal species is detected under stringent conditions. For example, probes mounting YPL088W-713 and OmpA shown in Table 1 below (SEQ ID Nos. 220 and 255 respectively) may be used. However, that is not the only option.

TABLE 1-1
Probe Sequence List

| Probe # | Symbol | sequence |
|---|---|---|
| 1 | A2m | gtagctggagaagggtgtgtctacctccagacatccttgaaatatagtgttctcccgagggagaa |
| 2 | Abca4 | tccagaacccctgactggtggctttggccttagcgatactttcattctgagtggatctgcttttg |
| 3 | Ace | tgaattacttcaagccactgacagaatggctcgtcaccgagaacaggagacatggagagacactg |
| 4 | Adam9 | ctctatggtacgaggtgtttagtatacccaagcagataggtgtcgatcgaacaggagcagggaga |
| 5 | Adipor1 | ccccttacccccgtccttactttgtaacctggctgataacgggccatccatttttgtagcacact |
| 6 | Agt | tttctgggcagagtgaataacccccagagtgtggtgtgaggccttgtgcctagccatggagacaa |
| 7 | Aif1 | gctgaagagattaattagagaggtgtccagtggctccgaggagacgttcagctactctgactttc |
| 8 | Akt3 | cgtctgtactgtctacatcacggttcccttagcttgctcctggtagtgcattacaggcaagcatg |
| 9 | Amy2a2 | gcctgacctgatagagcagggtgattctgctggagctatgggtcaagatatggagacctggaagg |
| 10 | Amy2a3 | gcctgacctgatagagcagggtgattctgctggagctatgggtcaagatatggagacctggaagg |
| 11 | Amy2a4 | gcctgacctgatagagcagggtgattctgctggagctatgggtcaagatatggagacctggaagg |
| 12 | Amy2a5 | ccaagaggtcattgatctgggtggtgaggcaattaaaggtagtgagtactttggaaatggccgtg |
| 13 | Apbb1 | attccttcacagaagtcattacactggactgatgacatgaggggccagaagcaaagccagccttg |
| 14 | Arr3 | gagttaagaggatccgatagcctatctctgataattctgtgtggaagcccccactgcaacactct |
| 15 | At1r | ggtgtgccctactcagtctccagaacagccctgtaggtccagccctctctgcaaccagagaaact |
| 16 | At2r | ctgcctgtcccatattataccaggtcacctaagaccttcctggattgatgctgacctatgaggta |
| 17 | Atp6ap2 | ggaaatacgcactgagagaactgtaaaccacttagtcatgttactcgcgctggagaacgcactgg |
| 18 | Best1 | gccgtttggaccagatgtcaaccaatatacaggctctaatgaaggagcatgcagagtcctatccc |
| 19 | Bmp4 | ttatcaggagatggtggtagaggggtgtggatgccgctgagatcaggcagtccagagggcggaca |
| 20 | C1qa | ctgtgcagtgcccggcttctattacttcaacttccaagtgatctccaagtgggacctttgtctgt |
| 21 | C1qb | ccccttgactgcctgaaacccagaccagagccctgtagatgttacagaacgaatgggtcaataaa |
| 22 | C1qc | cctgcggctccagaggggcgatgaggtgtggctatcagtcaatgactacaatggcatggtgggca |
| 23 | C1ql1 | agttggcgcggttgcttcctgtagggtgaccctgccttggggaggagtgggttaggggttggata |
| 24 | C1s | gcatggaggcagggttgatcactgagccgtgttggttattcagttactattgctaacaacatggc |
| 25 | C3 | agttcaccagtactttaatgtgggacttatccagcccgggtcggtcaaggtctactcctattaca |
| 26 | C4 | ctttgagtccaagatcacccaagtcctgcatttcagaaaggacaccatggcctccataggtcaga |
| 27 | Calb1 | cccaggatggggagaagtgatacactggctttacttcggcacgtatttggaagcagtgactttag |
| 28 | Calb2 | aacaagaaggagatgaacatccaacagctcaccacctacaggaagagtgtcatgtccttggccga |
| 29 | Casp14 | ccaaaaccccatgagctaggcctcaactaccacaaaacagcctatgagctctgtttccaggattc |
| 30 | Casp3 | ccggtggaggctgacttcctgtatgcttactctacagcacctggttactattcctggagaaattc |
| 31 | Casp8 | aagaactgcgtttcctaccgagatcctgtgaatggaacctggtatattcagtcactttgccagag |
| 32 | Casp9 | tttgctttgtgaaagtccacttgagtttagatggtagatgtgccaagccttgtttgtttattttt |
| 33 | Cckbr | ggcctaaaatcacatgcctttcggagccgcagatctcttagcactgaaaaagtcccgtcatccct |
| 34 | Ccl17 | atcaggaagttggtgagctggtataagacctcagtggagtgttccagggatgccatcgtgtttct |
| 35 | Ccl2 | cacttctgtaggagtgaccagtgtgacagtgaactagtgtgactcggactgtgatgccttaatta |
| 36 | Ccl3 | tctcctacagccggaagattccacgccaattcatcgttgactattttgaaaccagcagcctttgc |
| 37 | Ccl4 | agcagtctttgctccaagccagctgtggtattcctgaccaaaagaggcagacagatctgtgctaa |
| 38 | Ccl7 | cggtcctaagggataggagctgtctgtaggaatgtgaaaccgtcacgcctaaggcatggtcttta |
| 39 | Ccl8 | gctgctttcatgtactaaagctgaagatcccccttcgggtgctgaaaagctacgagagaatcaac |
| 40 | Ccnd3 | ttgagtccctcttctgtccggggctccaaccttctcagttgccaaaacgccccagtaccttccaa |
| 41 | Cd44 | ttgattgactaataataatgaggaaaacctgatgtgtacattacccgattgaaagtgtgtattgg |
| 42 | Cd45 | ggcgcatcagaaggggataaagaggactctgttttctcactagccactcacagatttctatctca |
| 43 | Cd55 | gtttaataaccttgacagttttgcatgtgatgctatcactcattggctacttgacatagccaacg |
| 44 | Cd59a | gagctggtgtgtgaatgttccagttgtgagctgtcagttaggggcagtgactgttcacttgctta |
| 45 | Cd74 | ctaccgctatctaaagggaacccccatttctgacccattagtagtcttgaatgtggggctctgag |
| 46 | Cdh1 | tggccgatttaaacccaagttgcccagttctgagtagaaaactgagactatgctgtgtgtggcgg |
| 47 | Cdh3 | gcctgggttacctatcacaactctgtctcagagaacagaaatactttccaggccagccagagctt |
| 48 | Cdh5 | gtcgaatttgaagcaactgtgaattcacccagggaggactgtggagaccataggtgacaggtcat |
| 49 | Cdr2 | cctgcagcgattcctgtcgactcccttacacagtattgatcttctgactgtggaaacaaccttcc |
| 50 | Cebpd | ggtgacagtgccacctctggcagctcccagaacactaaaaccacaaagtgtttaggttggacatt |
| 51 | Cfb | aggtgtacatcaagaatggggacaagaaagccagttgtgagagagatgctacaaaggcccaaggc |
| 52 | Cfh | ccgagactcaacagggaaatgtgggcctcctccacctattgacaatggagacatcacctccttgt |
| 53 | Chga | ccttcttatccatggggcacaactgcaataacttctgacctttgggcgaaagccgagaactcctg |
| 54 | Chrna7 | tcccagcagcttctgtttacttttcttacacccatcaccggcatatcattgtactcggcaggggc |
| 55 | Ckb | aagtccaagaactatgagttcatgtggaatcctcacctgggctacatcctcacatgcccatccaa |

TABLE 1-2

| | | |
|---|---|---|
| 56 | Cldn5 | cagactacaggcacttttaagaacttgaccgaccttttcttctatgcgcagttggccacgacgtg |
| 57 | Clip1 | gggaagacctgaacagttatgacagtgatgaccaggaaaagcagtccaagaagaaaccccgcctc |
| 58 | Cnga1 | ctcctgcaaacgcgatttgcccgtatcttggctgaatatgagtcgatgcagcagaaactcaagca |
| 59 | Cnga3 | ccaaacgtgtagtccttcccatatttattgaggtgaaggtcctgctctgttacccagtccagcct |
| 60 | Cntf | ctccgtgtcatttcttctcatcacatgggaatctcagcacatgagagccattatggggccaagca |
| 61 | Col7a1 | cctgaagatgacgatgacttctctgagtactctgtgtattctgtggaggactaccaggagcctga |
| 62 | Col8a2 | ttccccaggtcaaagccactaatccaatgggttctggcggtaagagattagtcaactcccagagg |
| 63 | Crx | ctagcctatcaggtctcccttccctggctacccagagtgaaactgattaaaaatgtggatcccac |
| 64 | Ctnna1 | attaacttcttggtcacatttctgagtaggctgaagtgcctgctttctaggtaggggtgagcgcc |
| 65 | Ctss | tctacaaaagcggtgtctatgacgacccctcctgtacgggcaatgtgaatcatggtgttcttgtg |
| 66 | Cxcl1 | ctgctctgatggcaccgtctggtgaacgctggcttctgacaacactatacaatttcttttgaggg |
| 67 | Cxcl12 | ggagccagaccatcctggataatgtgagaacatgcctagatttacccacaaaacacaagtctgag |
| 68 | Cxcl2 | ggatttcaatgtaatgttgtgagtaacccttggacattttatgtcttcctcgtaaggcacagtgc |
| 69 | Cyb5r1 | tgcgggaggacctagaggaacttcaggcccagtatcctaatcgctttaagctctggttcactctg |
| 70 | Darc | cctccctacaagacaggcttctcaaatggatgcccttgcaggcaagtcctagttttgttttgttt |
| 71 | Doc2b | gccatccaccaccgttagccaatccctgaacagtcccgtcttccaacccagagtcagtgacctcc |
| 72 | Edn2 | aggacacaattacctaagccacacctggagactgaacagtcatcctaatgactgacatcatggct |
| 73 | Ednrb | gaatccacgtgacacatgactctatttaggagtcacccacagttcttgtgtgtacagattgcttt |
| 74 | Efemp1 | gtctacaaatacatgagcatccgatctgacaggtccgtgccttcagacatcttccagatacaggc |
| 75 | Efna5 | ttcttgacaccgcattgaagccactcacctgtgtgcgtctgggtatgaagtccagctccttccgt |
| 76 | Egf | aaccaggctgatgatggtagagtgctacagacttggtactccagtttccacggctaatcactgct |
| 77 | Elovl4 | ccagaccccttcactccctgctctccgatttcagcacaataaatacactacagctgtgggaaaag |
| 78 | Eno3 | gttccgtaatccaaaggccaaatgaggagctggagactccaggctttcacaggaaagacacaggc |
| 79 | Erap1 | ggggcatggggcaacagtggaaacctgctgataccagtcatggtagtacttgtcattttcactga |
| 80 | Fgf16 | agtggtataggtgcagatcaatggtgaagtgcttcttagcatgggacgagccctagattccatcc |
| 81 | Fgf7 | acctatgcatcagctaaatggacacacagcggaggggaaatgttcgttgccttaaatcaaaaggg |
| 82 | Fgfr1 | tcaattctgccacctgatggttgctttggtaccttggtctcttattcaaacccacaccactcaag |
| 83 | Flt1 | ctatttggggtctagagatgagccctgggtctctaaaatggctctcttagaagttgtatgtgcaa |
| 84 | Gabrb3 | gtgacatgtccatctgcagtattgtgtgttttgatgccaacacggggacatcttcagttgttccc |
| 85 | Gem | gaaggagaagggctcactgcatacttatatccctgaaacgacatctttagagctggcctcatcac |
| 86 | Gfap | cttcacttcagtgctgattcagcccagagggttagttagttccctctggacggctgctcttgtag |
| 87 | Glut1 | ggattcgcccattcctgtctcttcctacccaaccactcaattaatctttccttgcctgagaccag |
| 88 | Gnao1 | cggtgaggtttctgtgacacctgacactgccagcctacttctctaatggattcctgtgtagctca |
| 89 | Gnat1 | gggtggctcagagcagagtccaacccctattgatcaaccatgggcaaagaatatcccatgtgagg |
| 90 | Gnat2 | gccagcgaccctaacgcccaggttccaaggctggttctgtttagtataagaacagaaaagcttca |
| 91 | Gngt2 | cctcctcagccacgcccaagtgtactgggatacctaaggaccaatgttttcttcctcttagacac |
| 92 | Gpnmb | ccaagtgaccctggtaagggaactgtctgcagaatggaagaaatagcctaagagacagggatggc |
| 93 | Gpr143 | caaaaccccaggaaggttgtatgtgtcgggggacatacttctgatgaggtgctgagcattttgtc |
| 94 | Grem2 | gtgggatggctctcacctccagtctatgggaacttcaagcctttattgaaacacccaccagctga |
| 95 | Grm2 | gagtctctgcattggccagtaactatccttgtagctatgccccgtgtttgccaggcctgggaatc |
| 96 | Grm6 | tttcccatggtgcagagcaccggtctacatttactgccgagtgcgatccaatgctctggactgag |
| 97 | Gsk3a | ggagtagagagagtccctggtgtcttagtttccacagtaaggtttgcctgtgtacagacctctgt |
| 98 | Gsk3b | ggtcagtttcacagggttatgccattttattcaagtccgttgctccgttgtgcagtctccaccat |
| 99 | Guca1a | tggagcgactcatccatgagtgccgaggaattcacagataccgtgtttgccaagatcgacatcaa |
| 100 | Guk1 | tgcttcattccacagagtgatgtctgtggtctaaatttgcctggaggtgggggaaactttgccag |
| 101 | H2-K1 | tcactggagctgtggtggcttttgtgatgaagatgagaaggagaaacacaggtggaaaaggaggg |
| 102 | Hfe | caggacccactcaactatcctccatctgttatagagtgactcctctgtcaccatgccctgacttc |
| 103 | Hif1a | atctgttcccattagcaggtgaaggaagstagggctgaaacaagagttttccgcgctctcaggga |
| 104 | Hspa1a | gtgcctgttgtgtttggaggtcaggagttgctgtgtatgacagtttcggctacgcttgactaaca |
| 105 | Hspa1b | tatcagtgttccagtagcctgggaagacatatagtctagctgcccagttccctggagatggtcat |
| 106 | Hspa2 | cgtatgtacatggagatttgcttgaaagtagaaccctgatgctcgcacacctgacctgtggaagc |
| 107 | Icam1 | ctacttttgttcccaatgtcagccaccatgccttagcagctgaacaatcgagcctcatgctcatg |
| 108 | Id3 | ttgctgctttaggtgtctcttttcctccctctctatctctactctccaacatgaaggcgctgagc |
| 109 | Ifna1 | ccaggaagatgccctgctggctgtgaggaaatacttccacaggatcactgtgtacctgagagaga |
| 110 | Ifnr | agctggctgcactccactagaattgccacgacgtacctatcacagtgacgtctacagtgagaaat |

TABLE 1-3

| | | |
|---|---|---|
| 111 | Igf1 | ggctccatccatatctcctatcggtgtatctgtatccttaaaccttgcaaacatcatacagtgta |
| 112 | Igf1r | tttctaagccagtgaggttgaggtgagaggtttgccagagtttgtctacctctgggtatcccttt |
| 113 | Igfbp3 | cagcatcggactccacgttcagagatgtcgcaaataatggtgcgcttagttcttcagatgacttc |
| 114 | Il10 | ctgatccagggatcttagctaacggaaacaactccttggaaaacctcgtttgtacctctctccga |
| 115 | Il17a | ttcctccagaatgtgaaggtcaacctcaaagtctttaactcccttggcgcaaaagtgagctccag |
| 116 | Il1a | ggaacatccttaaatcctctgagcttgacaggcatcctcacagcaggattttctaggtggtcagt |
| 117 | Il1b | cattaggcagcactctctagaacagaacctagctgtcaacgtgtgggggatgaattggtcatagc |
| 118 | Il6 | atctactcggcaaacctagtgcgttatgcctaagcatatcagtttgtggacattcctcactgtgg |
| 119 | Irf6 | gtagatacccagtccccaggagctctaagtttcccaatgctcccattcactctccagctgtcttc |
| 120 | Irs1 | ggtgctatcgacggtgctacttcatctgtgtacaaaagaccgacgcatggtctatgttgctacca |
| 121 | lsgf3g | ttgactacttggtcctcagtggaaccacttgggatgactagaggtgtggggttgttggttgagtt |
| 122 | Itgav | ttgccgccttacgagcacagtgtgagtttctgtagtgagtctaaccccgaggagggaatttcgtg |
| 123 | Jak3 | aaagccccatattttggtatgccccggagtccctatctgacaacatcttctcccgccaatctgac |
| 124 | Kdr | tggtctcactaccagttaaagcaaaagactttcaaacagtggctctgtcctccaagaagtggcaa |
| 125 | Lgals3 | gccttttaaactttgtgtgttgtgtgtctgtgcacatgggtacaggtgcctgctcacttgagagg |
| 126 | Lipc | tgacctccagcttcacccgagccaggagaaagtctttgtgaactgtgaagtgaagtcaaaaagac |
| 127 | Lmo1 | actatgaggaggggcatctcaatggcacctttgaatcccaggttcagtaacgcccaccatctggc |
| 128 | Loxl1 | gtgggaaaggtctaccttcccaaagagagaagacacatgcattcttggcagaggggatagcatgg |
| 129 | Mark2 | tatttctctgctgtttcagttcttgagaaattggggaagtcctacagaggctgcccctgccctca |
| 130 | Math5 | tacgagacactgcagatggcgctcagctacatcatcgcgctcacccgcatcctagccgaagccga |
| 131 | Mef2c | ccgcctctgccatctgctccggtgtcgtcagttgtatggcattaatttcctgccactagagatat |
| 132 | Mkks | gcttctccaagttgttccaagaaagggtgccctgtacctttccactggcaggacattaccattgt |
| 133 | Mmp14 | cctagttggctgcctcccgccactctgactaaaaggaatcttaagagtgtacatttggaggtgga |
| 134 | Mmp2 | cctcctctgtagttaaccagccttctccttcacctggtgacttcagatttaagagggtggcttct |
| 135 | Mmp8 | actggagggctgtatctataaatctatttgccaataagttcccaggcagaggcaggtaggagggg |
| 136 | Mmp9 | gggcgcggctccaaccgctgcataaatattaaggtattcagttgcccctactggaaggtattatg |
| 137 | Msr1 | agccaatgattttgggagtctgtcgtatgaatggactagttattctgtgaaaacatctagagaaa |
| 138 | Nes | tgggccagcactcttagctttgataacttgacctgtggtatctctcgtggagaggtgtggctggc |
| 139 | Nfkb1 | ccgcatttggcgtccttcttggttctgaaatgaaatgtagttgccacgcacagacggtgtctagc |
| 140 | Nos3 | atcctgctgccctcttcatatgctttgatggactgtagactccctttaactctctctctggcgta |
| 141 | Np | ggacccaccatttaagggggaaatgtgggtgagaggaaagtggagtactctcagctaacacacag |
| 142 | Nr2e3 | ggggcagcacatcttagaagctaaatagttccctgcctttctcagccagtaattccacattcagg |
| 143 | Nrl | gagttgggagtcgccacctcatcagctactgtcatctgtcctctaaggggcataataaatgggag |
| 144 | Nt5e | gactcaaatcccacacaaccactgtaaggcatactcaggtcaagacatgagaagaccagcaggcc |
| 145 | Opa1 | gtgaccaaatggaaagggttggtgtggaagcacgtgagacttcctaactgtacagcgtgggatgt |
| 146 | Opn1mw | gcctccaacctgtcccatccatcaaagctttggccatgttttacctcccttctcatctatccttg |
| 147 | Opn1sw | aaggagcccctcgacacgaagaagtgcgtgttgagttatacgcaaggcgtgctgggtactgatat |
| 148 | Osbpl1a | ctaacccctacagtggagcacaggactggatttattctggcagctactgggacagaaactacttc |
| 149 | Pax6 | acacaggctgttggatcgcggatctgtgttgctcatgtggttgtttaaaggaaaccatgatcgac |
| 150 | Pde6a | ccttcaggggtttcctcagctgcaggatattctgcatttttcagggggctctatgcctgagatgg |
| 151 | Pde6b | cagaagaaggaagcagacagagtcgcagccacgaaagtgggcacagcagtttgcaatggtggtcc |
| 152 | Pdgfb | cacactgctgtccccatgacctccatttcccaaagtcctctgctccagcaactgcccttccaggt |
| 153 | Pdpn | gttgctgggacagcgctgtagtgtcttggtcgtctgtgttggtacccaatacagtgtagacatct |
| 154 | Pecam1 | tttattccccactaaagaaacggtttcctaaggtctgagctgtttcccagggtgggctagagtgg |
| 155 | Pex1 | taccggagccaaagtggagaggatgaatcccttaaccagcctggaccaatcaaaaccacttttgc |
| 156 | Pgf | caccggttgtctctgccgggactaactgccaagccagattctcttgaataaagcattctagtctg |
| 157 | Pig7 | catgctggttaagatgcagcctagccattgcctgagctgttcatcccatgatccggaagtgctcg |
| 158 | Pkia | gcctggaaatgttctttcactgacttgtggcagcctgggaagtcatctgtgagcttctatgactt |
| 159 | Polg2 | aacttcacccttgtttagcccctattaaggtcgctttggatgtggggaaaggcccaacggtagaa |
| 160 | Ppara | gatgcagagggctgagcgtcggtcatgcgggctctccccacatcctttctgaatgggcacttcta |
| 161 | Prkca | cctctactctccgtttgcatgatccgctctgttagatgcattcattgtagttcggaagcaagcgt |
| 162 | Prnp | gcgtgcactcagttccgtaggattccaaagcagacccctagctggtctttgaatctgcatgtcct |
| 163 | Prom1 | cacaagggatcttgagagaaggaactgtcgctcagctgggagcggaatcattatcgcaatcacag |
| 164 | Ptgds | cctcaatctcacctctaccttcctcaggaaaaaccagtgtgagaccaagatcatggtactgcagc |
| 165 | Pxmp3 | ggcaagagagttctggagactgtgggacagctaggatgaagaactctatgttataagcctctgtc |

TABLE 1-4

| | | |
|---|---|---|
| 166 | Pygm | tacaaagtccacatcaaccccaactcgctctttgacgtccaggtgaaaaggattcatgagtacaa |
| 167 | Rcv1 | gaggaggaattcatcgaggggaccctggccaataaggaaattctgcgactgatccagtttgaacc |
| 168 | Rdh9 | ttgtgtgtacctgtatggtacatgtgtgggagagccttcagagatgggaaggtgtggagagccgt |
| 169 | Ren | gcaagttctatacagagtttgatcggcataacaatcgcattggattcgccttggcccgctaaggc |
| 170 | Rho | cctgcccttgaggggattatatgagatttaagggacttatgtggccagcctacttcctggcatgc |
| 171 | Robo4 | cgcagggaaacagggaaccaatgcgctattctcattctaccgccactctgagcttaaggcactta |
| 172 | Rom1 | aattcgttactcagactcagggaaggaaaggtccctggggttataggagttaagagcaagcggag |
| 173 | Rpe65 | agggcaaaagcctgcatatctcctggttctgaatgcccaagacttgagtgaaattgccagggctg |
| 174 | Rs1 | gggctttgacaccctcctcctcgctgcccgaaagacagtcttctggctacctttggatcaagata |
| 175 | Rxrg | ggtggagttttgtacggctgttaaaggtggcccttctttgctatttaaggggctgaggtatttcc |
| 176 | S100a6 | ggctgatggatgatctggaccgtaacaaggatcaggaagtaaacttccaggagtatgtcgccttc |
| 177 | Sag | gccttgagggaagcccccggtagactctcaaagttatgctagatatcaaagcatgagcattcctc |
| 178 | Scd1 | caacccaagaaactcctgggctaagtatctgacagtctcacatctcaacagtgtgaattaagtgt |
| 179 | Sdc2 | ggcccttccaaagctgcacaatgcccctcaattacggaggctgagaatgtagtgggtatacctct |
| 180 | Selenbp1 | atgaccgcttcctttacttcagcaactggctgcatggggacattcggcagtatgacatctctaac |
| 181 | Selenbp2 | atgaccgcttcctttacttcagcaactggctgcatggggacattcggcagtatgacatctctaac |
| 182 | Serpina3n | ttatagccaagatagccaaccccaaatgagactagaactccccaagtgttgacgcttcttcccgg |
| 183 | Serpinf1 | gaatcctggaccccagtagtacttaatgtctcagtgctctacagaacccccagagggaagctgat |
| 184 | Serping1 | ttcctcttcctgctctgggaccagcaacacaggttcccagtcttcatgggtcgtgtatatgaccc |
| 185 | Sfrp5 | tcacggccgtctaccgctgggacaagaagaataaggagatgaagtttgcagtcaaattcatgttc |
| 186 | Sil1 | acctgctggggcatgagtgtcacctgggcagagcttgctaggtcattaaacagagacatgatgac |
| 187 | Slc16a1 | ccttgtccgtcactcctttgaatgctgattttgtctcaagtcctttcagttcacatagccgtggc |
| 188 | Slc16a4 | ccactggctgaaagatggaaacgcaaacagtctgaccttctgaggactacaatcaagtaagagcc |
| 189 | Slc1a3 | cacttcctcttcagtccctaatcagcccccaaagaaacgacgtcataacaccacttcctcctcat |
| 190 | Socs3 | aggagactcctgagttaacactgggaagacattggccagtcctagtcatctctcggtcagtaggt |
| 191 | Sox9 | ctgctcagactatcacctgtacctccctgaataccagcgtttaaccttcaagacatcccatgtgc |
| 192 | Sparc | cacctctccccaccccctgccacttgaaaccttctactaatcaagagaaacttccaagccaacgg |
| 193 | Spp1 | caacaacggaaagggcagccatgagtcaagtcagctggatgaaccaagtctggaaacacacagac |
| 194 | Statl | tcaccgttttgagggatgatgttttgtggcacgtgtgtgatcacagcctgatggttctggtcgtg |
| 195 | Stat3 | gaacttataaactgaaagggtatttaggaaggcaaggcttgggcatttttatggctttcaatcct |
| 196 | Stat5a | aggtttaggcaactaagttggagttttactcctaagctagaagcttcgcccagaccggtgtgctc |
| 197 | Stat6 | ctcatgcaggtgccttccgtctcaactgttccttggttaagagaaaagaactggctgggagacca |
| 198 | Synpr | gtccaatgaccctgtgcttgtagagtggcgttttctttgcatccagagaggcagatttagacacc |
| 199 | Tgfb2 | agctacctgggtccattcctcccccaaccccagttccttctattttccaaaagataaaaaccaaa |
| 200 | Tgfb3 | aatcatatcttgcactgcctggaattaaggacaatccgttctttctgcaactgtcttttcacctc |
| 201 | Tgfbr2 | gaatgctggtccactcgtgggatttctagggttcaaaagtgacttcacttccgggtcatcatcag |
| 202 | Timp1 | gtgaagagtttctcatcacgggccgcctaaggaacgggaaatttcacatcaatgcctgcagcttc |
| 203 | Timp2 | cctccctcccttactcccgtcatgccagcaactcgcaatatttcagatgacgtttacatggtagc |
| 204 | Timp3 | tagatctaagtcagctgtttgggttgaggaggagagaacccgaggaaatgaccatgctctgggga |
| 205 | Tlr4 | ggggaggaagaaaggtctaacatccttttccttcatcattctcatttctggacatgccttgtgag |
| 206 | Trip1 | aagctcttcgactccacgactcttgaacatcagaagactttccgaacagagcgtcctgtcaactc |
| 207 | Ttpa | ccttcagtgtctttgctagatcaagtgcagacgctgcacacaatctctagttcctctagttctgg |
| 208 | Tyrp1 | cgaatggctaaggaggtataacgccgatatttctaccttcccgttggaaaacgcacctattggac |
| 209 | Usp9x | gtttgcagaagtatgtagcacttgtcctccaagctttcaggtgtaagggggaaaggtgaaccaca |
| 210 | Vcam1 | tgatcccttgctgaatgcaaggagctaaccagaaaagttctgcttgacaagtccccatcgttgaa |
| 211 | Vegfa | ctagcttgtcctgagaagatatttaattttgctaacactcagctctgccctcccttgtccccacc |
| 212 | Vegfb | aacaattgtcaaggaacctcatgtctcacctcaggggccagggtactctctcacttaaccaccct |
| 213 | Vegfc | ccaagtctgtgtttattgaaccatgtggattactgcgggagaggactggcactcatgtgcaaaaa |
| 214 | Vegfd | gacaaatgacttgtagcttcagatgtctttgcgccatcagcactcagaaaggaaggggtctgagg |
| 215 | Vldlr | cgctgtcttagtactgtcactgttgtaaaggcacatgttggaacacatccagtcctgctgacctg |
| 216 | Vtn | gcgtctatttcttctctggagacaaatactaccgagtcaaccttagaacccggcgagtggactct |
| 217 | Actb | ccatcgtgcaccgcaagtgcttctaggcggactgttactgagctgcgttttacaccctttctttg |
| 218 | GAPDH | gggctgccatttgcagtggcaaagtggagattgttgccatcaacgaccccttcattgacctcaac |
| 219 | Arbp | atcagatgaggatatgggattcggtctcttcgactaatcccgccaaagcaaccaagtcagcctgc |
| 220 | YPL088W-7 | gcatgttgactcgtcctctgaaccaaagcacggacaggattaagagtgatccaactttcaagtcg |

TABLE 1-5

| | | |
|---|---|---|
| 221 | cxcr4 | gcaggacctgtggccaagttcttagtagctgtttatctgtgtgtaggactgtagaactgtagagg |
| 222 | esm1 | acttgattgatgaatggacctgagtttttacccggaggaccttagcacaagaagaactgttgtcc |
| 223 | apln | gtccctgtgcccacagattgcacgtgtagggaggtgagtgcttgtatcccaaattggttctaggt |
| 224 | egln3 | ccccaactttcaaacctctttaatttcctagctagaatcccacaagcagcatagacctgaaagtg |
| 225 | Cp | cctgctcttcgtgttctctgccctgctggagtacgccgccgtcaactttgtgtctcggcaacaca |
| 226 | Tnfa | ctgtcgctacatcactgaacctctgctccccacgggagccgtgactgtaatcgccctacgggtca |
| 227 | Tgfb1 | cccgtgcagagctgcgcttgcagagattaaaatcaagtgtggagcaacatgtggaactctaccag |
| 228 | IL6r | aatcctctggaaccccacacaggtctctgttgaagactctgccaaccacgaggatcagtacgaaa |
| 229 | Tnfrsf1a | ttcaaggaccattctgctagatgccctactccctgtgggtgaanagtgggcaaaggtctctaagg |
| 230 | Aoc3 | gctggtacacacatgactcaaaacaacagatacatcattccctagcagtctggccagccccgttg |
| 231 | Ptgs1 | gtcaaggcagtaaggtgttcttgggagccacacttagactctttccaaagatgtggagggaacag |
| 232 | Ptgs2 | ggctgttggaatttacgcataaagcagactgcatagatccaatattgactgacccaagcatgtta |
| 233 | Pla2g5 | ctaggcttcttcaaatggagctccagaatgtgcacgggacacatccgatctatatttagagacct |
| 234 | Il6st | tgaacgttgcagtgtgaagtctgtactggctagggatggtagcccagcgtgagttcaggcatgaa |
| 235 | Nrp1 | aagcagcttccggctggtagatttttgtcttgatgggctgtccataagcactcccagttctttcc |
| 236 | Mapk1 | aaataaagtggcagccattagacactgtgacggtcgtgtgcattgtgggagtggacttttatgga |
| 237 | Mapk3 | cctattcatcttgttggcaccccaccccattttccctgacagaacattcctaagtctcaagggct |
| 238 | Jun | ccctgtctgatttgtaggaatagataccctgcatgctatcattggctcatactctctcccccggc |
| 239 | Fos | aaacacgtcttccctcgaaggttcccgtcgacctagggaggaccttacctgttcgtgaaacacac |
| 240 | Tlr3 | ttttgtgcctcaaagttcggttggggctaccttgggatcccaaacagctaatatggtcaccaact |
| 241 | Ctgf | gtggggggcagtttatttgttgagagtgtgaccaaaagttacatgtttgcacctttctagttgaa |
| 242 | Epo | taggcgcggagatgggggtgcccgaacgtcccaccctgctgcttttactctccttgctactgatt |
| 243 | Ccr2 | gtagtgaatgaccaagaataaggagaaaagccaactccttcatcaggcatagagagctgcagcaa |
| 244 | Angpt-1 | cttgataaccgcagccacaaagccttagtgactttcctctacctggtaagacagagctcttcatg |
| 245 | Angpt-2 | cagaacacttagatggtgcagataaatcttgggaccacattcctctaagcacggtttctagagtg |
| 246 | Adipor2 | gcctccgggaactttccaggttggcacctgaatgccttactctcagcagtctgaggctcgcttgc |
| 247 | Adam28 | atagtgcacaaggatggtgggaataaatgaagcatcccacactcaacctcccaacatccataact |
| 248 | Adam17 | gctgtggcattgatgatacctgcctcagtcaataaatactgaatatcaaagcagtggattgcgta |
| 249 | Adam19 | agcctagcaccccaaagtcatgcacccagtatcctcttgtatgactgtatatgtctatgtctggg |
| 250 | Mmp1 | gcaagccagaataaagactgtgccagctggtcagtcgcccttttgagaccactcctttgtgatcc |
| 251 | Mmp7 | ggacgacattgcaggcattcagaagttatatggaaagaggaacacgctgtgatagatgcagacag |
| 252 | Selp | ccagatggtactaggggacacagaagatctggaacagagccaagacgtcacatctgactgcagac |
| 253 | Sele | gtcctggcactgaagccagcatgagatccatcattcttatgtcagctcaagggtcaaaaggactt |
| 254 | Furin | cccgatgctgctttcccctgtggggatctcaggagctgtttgaggatatattttcactttgtgat |
| N.C. | OmpA | gtgtcggcataagccgaagatatcggtagagttatattgagcagatcccccggtgaaggatttaa |

### 4. Microarray

A microarray is where numerous probes are immobilized densely but independent of each other on a carrier. An embodiment of the present invention provides a microarray with mounted probes to detect at least one type of a gene selected from the gene clusters above. Also, the embodiment provides a microarray to evaluate disorders in the periphery of a retina, disorders in the retina, light damage to the retina, inflammation in the retina, disorders in the retinal pigment epithelium or choroid, light damage to the retinal pigment epithelium or choroid, or inflammation in the retinal pigment epithelium or choroid. The microarray related to the present invention is not limited specifically as long as the probes of the embodiment (for example, oligonucleotide probes) are immobilized on a carrier. More specifically, the embodiments of the present invention provide a microarray with mounted probes having at least one type of base sequence shown in SEQ ID Nos. 1˜291 and 221˜254.

The carrier of a microarray is not limited to any specific shape, and a flat plate, a rod shape or beads may be used. When a flat plate is used as a carrier, predetermined probes are immobilized by type at certain intervals (for example, spotting methods or the like; refer to Science 270, 467˜470 (1995), for example). In addition, predetermined probes may be synthesized by type at specific positions of the flat-plate carrier (photolithographic methods or the like; refer to Science 251, 767˜773 (1991), for example).

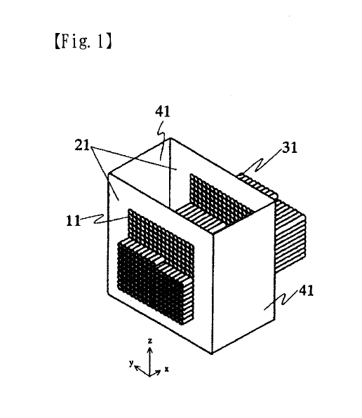

Another preferred example of a carrier is one that uses hollow fibers. When hollow fibers are used as a carrier, oligonucleotide probes are immobilized by type in hollow fibers, and all the hollow fibers are gathered and immobilized. Then the fibers are cut repeatedly in a longitudinal direction to obtain a nucleic-acid microarray. Such a nucleic-acid microarray is described as a type with oligonucleotide probes immobilized in a substrate with through holes, and is referred to as a so-called “through-hole type microarray” (refer to FIG. 1 in Japanese Patent No. 3510882, for example).

The method for immobilizing probes on a carrier is not limited specifically, and any binding method may be employed. Also, it is not always necessary to immobilize probes directly on a carrier. For example, a carrier is coated in advance with a polymer such as polylysine, and probes may be immobilized on the coated carrier. Moreover, when a tubular body such as one having hollow fibers is used as a carrier, probes may be immobilized on a gelatinous material held in the tubular body.

In the following, producing a microarray is described in detail by referring to a through-hole type nucleic-acid microarray using hollow fibers. The microarray is prepared by the steps (i)˜(iv) below.

(i) a step for producing an array by arranging multiple hollow fibers in a three-dimensional formation in such a way that the hollow fibers have the same longitudinal direction;
(ii) a step for producing a block body by embedding the array;
(iii) by introducing a gel precursor polymerizable solution containing an oligonucleotide probe into the hollow portion of each hollow fiber of the block body and initiating polymerization reactions, a step for holding the gelatinous material containing the oligonucleotide probe in the hollow portion;
(iv) a step for slicing the block body by cutting in a direction that intersects the longitudinal direction of the hollow fibers.

The material for the hollow fibers is not limited specifically, but the material described in JP2004-163211A or the like is preferred.

The hollow fibers are three-dimensionally arranged to have the same longitudinal lengths (step (i)). Such methods are, for example, arranging in parallel multiple hollow fibers at certain intervals on a sheet material such as an adhesive sheet, and having them set as a sheet, and then the sheet is rolled into a spiral shape (refer to JP H11-108928A); and laminating two porous plates each having multiple holes at certain intervals in such a way that those holes correspond to each other, passing hollow fibers through the holes, preliminarily fixing the two porous plates to have a distance between them, filling curable resin material around hollow fibers between two porous plates and curing the resin material (refer to JP2001-133453A).

The arrayed body is embedded so as not to disturb the array (step (ii)). Examples of such a method are filling polyurethane resin or epoxy resin into spaces among fibers, adhering fibers to each other by thermally fusing fibers, and the like.

In the hollow portion of each hollow fiber of the embedded array, a polymerizable gel precursor solution (gel-forming solution) containing a probe is filled and polymerization reactions are initiated in the hollowed portion (step (iii)). Accordingly, a gelatinous material with the immobilized probe is held in the hollowed portion of each hollow fiber.

A polymerizable gel precursor solution is a type of solution containing reactive substance such as gel forming polymerizable monomers, and when the monomer is polymerized or crosslinked, the solution becomes a gel. Examples of such monomers are acrylamide, dimethyl acrylamide, vinyl pyrrolidone, methylene bisacrylamide and the like. The solution may include a polymerization initiator or the like. After the probe is immobilized in the hollow fibers, the block body is thinly sliced by cutting in a direction that intersects the longitudinal direction of the hollow fibers (preferably at right angles) (step (iv)). The thinly sliced pieces obtained above are used as a nucleic-acid microarray. The thickness of the array is preferred to be approximately 0.01 to 1 mm. The block body may be cut by using a microtome, a laser or the like. Preferred examples of a through-hole microarray are a nucleic-acid microarray (Genopal) made by Mitsubishi Rayon Co., Ltd. and the like. In the evaluation method of the present embodiment, those microarrays may be used.

### 5. Eye Disease

In the embodiments of the present invention, “eye disease” means a disease associated with the eye. For example, diseases of the eyelid and lacrimal apparatus such as hordeolums, chalazions and neonatal dacryocystitis; diseases of the conjunctiva such as conjunctivitis and pterygium; diseases of the cornea such as corneal infections and corneal endothelial disorders; diseases of the uvea (iris, corpus ciliare, choroid) such as uveitis and Behcet's disease; diseases of the retina such as diabetic retinopathy, retinal detachment, retinal vein occlusion, central serous chorioretinopathy, age-related macular degeneration and retinitis pigmentosa; cataracts, glaucoma, optic neuropathy or the like. However, those are not the only examples. In the present embodiment, preferred examples of an eye disease are disorders of the retina, retinal pigment epithelium and choroid, more preferably age-related macular degeneration.

The above eye diseases include disorders of the retinal periphery. “The retinal periphery” indicates portions that include the retina composed of retinal arteries, retinal veins, macula flava, optic disk and central fossa, and portions that include the retinal pigment epithelium, choroid and sclera. “Disorder” includes damage and dysfunctions observed in those portions. They include, for example, cell death, inflammation, angiogenesis, accumulated waste products containing protein and lipids, and dysfunction of cells with light-sensitive receptors. Light damage is one of the disorders caused by light, and includes cell death, inflammation and dysfunction of cells with light-sensitive receptors. Also, since angiogenesis is observed when oxygen deficiency occurs in the retina caused by bleeding, the above eye diseases include those associated with angiogenesis. Examples of eye diseases associated with angiogenesis are diabetic retinopathy, age-related macular degeneration and the like. The “condition” of an eye disease indicates a particular condition associated with the eye disease. Examples of such conditions include the disorders listed above.

### 6. Subject Organism

In the embodiments of the present invention, a “subject organism” indicates a target organism that contacts, ingests or is administered a test substance, and includes individual animals, animal tissues and animal cells (including cultured cells). The animals are preferred to be mammals; and the mammals are preferred to be rodents such as mice, rats, rabbits and hamsters, monkeys, hogs, cats, dogs, cows, horses and humans. However, when subject organisms are used for experimental purposes, they should be nonhuman mammals, preferably rodents. The cells used in the embodiments are not limited specifically, and examples are cell lines from mammals such as humans, mice and rats as well as cell lines derived from those cell lines, primary cell lines from various tissues, stem cells, ES cells, iPS cells, and the like. The cells to be used in the embodiments are preferred to be those related to the eyes. Examples are retinal cells (visual cells, nerve cells), pigment epithelial/choroid cells and the like.

### 7. Method for Evaluating Condition of Eye Disease in Subject Organism

The present invention relates to a method for evaluating the condition of an eye disease in the subject organism by detecting a particular gene in a sample taken from a subject organism and comparing the detection results with a control. The aforementioned microarrays may be used in the methods of the embodiments.

In the embodiments of the present invention, regarding a gene in a sample taken from a subject organism, the condition of an eye disease in the subject organism is evaluated by comparing the expression level of the gene of a normal subject organism (a control) and the expression level of the gene of a subject organism affected by the eye disease, and by checking to see if variations (increase or decrease) are observed in the gene expression level or if the gene itself is expressed or not.

Also, the same method may be used to evaluate disorders, and conditions of inflammation, angiogenesis and age-related macular degeneration in the retina and retinal pigment epithelium/choroid of the eye of the subject organism.

7-1 Sample Taken from Subject Organism

A sample taken from a subject organism means a bio-sample isolated or taken from the subject organism. Samples include part of the eye such as the retina, retinal pigment epithelium and choroid, bodily fluids such as blood, other tissues or parts of organs, homogenates, cell homogenates, or nucleic acids retrieved from such samples. As a sample for continuous measurement, cell homogenates and body fluids such as blood are preferred. When conditions of an eye disease are directly checked, the retina, retinal pigment epithelium and choroid are preferred.

In the embodiments of the present invention, a control means a measurement result obtained from a control organism to be compared with a measurement result obtained from a subject organism and to have a determination made accordingly. For example, a control to be compared with measurement results regarding the condition of an eye disease in a subject organism is the result obtained from a subject organism that is not affected by the eye disease. Also, a control in the method for evaluating inhibitory or restorative functions of a test substance for the eye disease is a measurement result in a subject organism that has not contacted, ingested or been administered the test substance.

### 7-2 Extraction of Nucleic Acids

The level of mRNA in a sample is measured by extracting nucleic acids from the sample. Extraction of nucleic acids and treatment of the extracted nucleic acids are conducted by using a method that is suitable for measuring the expression level of the gene.

Extraction of mRNA is conducted by the method below, for example; however, that is not the only option.

First, to an organ or cells frozen at −80° C. or by using liquid nitrogen, a 4M guanidine thiocyanate solution is added to make a total amount of 0.5 g/10 mL, and homogenized. Then, 1 mL of 2M sodium acetate, 10 mL of phenol, and 10 mL of chloroform are added and stirred well. The mixture is centrifuged for 10 minutes at 10000 rpm, and the aqueous phase is recovered. Next, an equal volume of isopropanol is added to the mixture, further centrifuged for 10 minutes at 10000 rpm to precipitate RNA. The mixture is dissolved again in 10 mL of 4M guanidine thiocyanate solution, to which 1 mL of 2M sodium acetate, 5 mL of phenol, and 1 mL of chloroform are added and stirred well. The aqueous phase is recovered in the same manner as above, and an equal volume of isopropanol is added to precipitate RNA. To the precipitate, 20 mL of 70% ethanol is added to form a suspension, which is then centrifuged to precipitate RNA. The precipitate is dissolved in 5 mL of a TNES buffer (0.1M Tris-HCl (pH7.4), 50 mM NaCl, 10 mM EDTA, 0.2% SDS), and Proteinase K is added to make a total amount of 200 μg/mL The mixture is reacted at 37° C. for 30 minutes, extracted by using acidified phenol and chloroform, and the extract is precipitated with ethanol. As for other methods, a commercially available reagent kit such as an RNeasy mini kit (QIAGEN) may be used. In that case, the RNA is extracted according to the supplied protocol.

### 7-3 Measuring Nucleic Acids

In the analysis method using the microarray, the mRNA level of a gene is determined as follows: mRNA, cDNA or cRNA (aRNA) derived from a bio-sample is hybridized in a microarray in which probes capable of hybridizing with a particular gene are densely synthesized and immobilized on a chip. The aforementioned microarrays may be used in the present embodiment. Especially a nucleic-acid microarray manufactured by Mitsubishi Rayon Co., Ltd. (Genopal™) is preferred because it is capable of densely immobilizing oligonucleotide probes by using polymer gels.

In the nucleic-acid microarray made by Mitsubishi Rayon (Genopal™), the oligonucleotide probes complementary to each corresponding sequence are densely immobilized. Using a T7 oligo dT primer from the RNA derived from a bio-sample, the double-strand cDNA is synthesized, and then aRNA is synthesized by in vitro Transcription. Biotin is incorporated at the time of aRNA synthesis, and the sample is labeled. The biotin-labeled aRNA is fragmented and hybridized to a DNA array. After the hybridization, aRNA is detected by streptavidin or the like modified with a fluorescent dye. The fluorescence is identified using a fluorescence detector, the resulting array images are quantified, and the value is set as the expression level of mRNA. Moreover, nucleic acid microarrays to be used are not limited to the above, and the same analysis may also be conducted using several other nucleic-acid microarrays available for a person skilled in the art.

### 7-4 Analysis of Measurement Results

As data to be used to evaluate the measurement results, values of the expression level having at least a determination value are used. As the determination value, an average value X of a negative control may be used. In addition, a value obtained by adding the standard deviation a to X is preferred, more preferably a value obtained by adding X+2a, even more preferably a value obtained by adding X+3a. A negative control is a gene that should not be detected in the sample from the test organism. For example, a negative control is a gene of an organism which is a different species from the subject organism, and is a probe with a sequence that will not hybridize under stringent conditions with the sequence of a complementary strand mounted in other probes (N.C. in Table 1). The error between the data of the obtained samples is corrected by the expression level of housekeeping genes (gapdh, actin, arbp, etc.). Variations in the expression level are determined by the corrected data. Variations in the expression level are statistically determined through calibration. From each subject organism, at least n=3 samples are acquired, and t-calibration is carried out. If value P is 0.05 or less, or if value P is even lower, such as 0.01 or less, it is determined that the level shows significant variations (increase or decrease).

### 7-5. Evaluation of Condition of Eye Disease

In the embodiments of the present invention, to assess the condition of an eye disease, the gene expression data in a subject organism that is not affected by the eye disease are compared with the gene expression data in a subject organism that is affected by the eye disease. The gene expression data to be compared may be obtained from the same subject organism or from multiple different subject organisms. Alternatively, data stored previously in a database may also be used. In the present embodiment, the expression level may be measured by using samples derived from multiple subject organisms. Thus, the expression level is measured in a predetermined number of test organisms (primary population), which is set as basic data values so as to be compared with the expression level obtained from the samples of one or more subject organisms. Making comparisons with a control not only includes comparing the increase or decrease in expression level, but also includes comparing the presence or absence of values. For example, it includes comparing samples regarding a gene that is not expressed in the basic data but is expressed in a subject organism.

Also, data on expression levels are processed again (to obtain average value, etc.) by incorporating the data from the subject organism into the values of the primary population so that the number of cases in the subject organisms (primary population) is increased. By increasing the number of cases, accuracy in the critical value of the expression level is improved, and detection accuracy is also improved by properly modifying the critical value.

Furthermore, a database is created by storing the measurement results of gene expression levels of the same type of subject organisms so that changes in the gene expression levels may be evaluated as a pattern. Such a method can be carried out by using a multivariate analysis, for example, principal component analysis, factor analysis, discriminant analysis, quantification theory (type I, type II, type III, type IV), cluster analysis, multi-dimensional scaling method (MDS), multiple regression analysis, conjoint analysis, comparison of the pattern using the Mahalanobis-Taguchi system (MT method), and a prediction of the effects.

The embodiments of the present invention are also capable of providing a database for storing such data as well as providing an analysis device that can read data and a program necessary for comparative analysis to execute analysis. Using such an analysis device, the condition of the eye disease in a subject organism is evaluated by a simplified method such as retrieving the stored data from the database and comparing the stored data with the measured data obtained from the subject organism.

In the embodiments of the present invention, the following formula is used to evaluate the condition of an eye disease.

By using the following formula, variations in the gene expression and the condition of a disease are determined quantitatively. Thus, it is possible to evaluate whether the condition of retinal epithelial cells and choroid is normal or not, and furthermore, what is the condition of the disease. Namely, by using the following formula, variations in the gene expression level and the condition of a disease can be associated.

M=(Xli×η1/β1+ . . . +Xki×ηk/(βk)(/η1+ . . . + [symbol] k) determination formula

When M1 max+σm1<M2 min is satisfied, the condition of the evaluation sample cluster is the same as that in each disease model.

In the formula, “M” represents the Mahalanobis distance to show the distance from the base space, “Xni (n=1˜k)” is input data for each sample and represents the gene expression level or gene expression ratio, and “ηn (n=1˜k)” indicates “η=(Sβ−Ve)/Ve/r, representing the (S/N) ratio in each sample. Each term is as follows: Sβ=L2/r; variation in a proportional term, Se=ST−Sβ; error variation, Ve=Se(/1−1); error variance, ST=X2i1+X2i2+ . . . +X2i1; total variation.

“βn (n=1˜k)” indicates β=L/r, representing the sensitivity of each sample. Each term is as follows: L=M1Xi1+M2Xi2+ . . . +M1Xi1; linear equation, r=M21+M22+ . . . +M21; valid divisor.

“M1” represents a normal sample cluster or a control cluster. “M1 max” indicates the maximum value of “M1” and “σm1” represents standard deviation from “M1”. “M2” represents an evaluation sample cluster, and “M2 min” indicates the minimum value of “M2”.

### 8. Method for Evaluating Inhibitory or Restorative Function of Test Substance for Eye Disease

The present invention relates to a method for evaluating inhibitory or restorative functions of a test substance on an eye disease in a subject organism by detecting a particular gene that is present in a sample taken from the subject organism that has contacted, ingested or been administered the test substance, and by comparing the detection results with a control result. The method related to the present invention can be carried out by using the above-described microarrays.

In the present invention, to evaluate the inhibitory functions of a test substance on a gene included in the gene clusters, the gene expression level obtained from a subject organism that has contacted, ingested or been administered the test substance in advance and is affected by an eye disease is compared with the gene expression level obtained from a subject organism that has not contacted, ingested or been administered any test substance and is affected by the eye disease.

In addition, to evaluate the restorative effects of a test substance on a gene included in the gene clusters, the gene expression level obtained from a disease-affected subject organism after it contacts, ingests or is administered the test substance is compared with the gene expression level obtained from a disease-affected subject organism that contacts, ingests or is administered no test substance.

Furthermore, inhibitory or restorative functions of a test substance on disorders of the retina and retinal pigment epithelium and choroid, and on inflammatory conditions and angiogenesis, may be evaluated in the same manner as above, and inhibitory or restorative functions of a test substance on age-related macular degeneration may also be evaluated.

In the embodiments of the present invention, contact, ingestion or administration means local administration (to the eye), oral administration, intraperitoneal administration or intravenous administration, if the subject is an animal. If the subject is cells, the test substance is added to the culture liquid and the cells are cultivated accordingly.

In the embodiments of the present invention, a “test substance” means a substance that a subject organism contacts, ingests or is administered, including food and drugs.

Food means all food and drinks—fresh food, processed food, beverages, condiments, processing materials such as food additives, homogenized plants, extracts from plants, mixtures thereof, food ingredients and the like. Pharmaceuticals include drugs, quasi-drugs, drug candidates, drug mixtures, cosmetics, cosmetic ingredients, fragrances, coloring agents and the like.

The concentration level when a test substance is added or the concentration level for a test substance to be ingested can be selected at any concentration, but it is preferred to be carried out at a concentration that does not cause toxicity in the subject organism so as to avoid any impact for extraction of nucleic acids. No toxicity indicates, in the case of animals, that death, partial necrosis, inflammation and the like are not observed; in the case of cells, release of inflammatory cytokines is not observed, or the cell survival rate is 90% or greater.

In the present invention, if the subject organism is a cell, the concentration of cells is not limited specifically, but it is preferred to be such a level at which sufficient nucleic acids are collected at the time of cell harvest, that is, final concentration of at least 1.0×10⁵ cells/well, more preferably a final concentration of at least 5.0×10⁵ cells/well.

The inhibitory or restorative functions of a test substance for an eye disease are evaluated by measuring variations in the expression level of each gene among a sample taken from a subject organism that has contacted, ingested or been administered the test substance, and a sample taken from a subject organism before it contacts, ingests or is administered the test substance or a sample taken from a subject organism that has contacted, ingested or been administered a blank (placebo or substance containing only a solvent).

Data pertaining to which gene of which group showed a change in expression level are analyzed, and the analysis results are used to determine the effects of a test substance on inflammation in the retina, angiogenesis or the like. If the test substance is observed to be effective, effects that can be expected from the test substance are also evaluated and predicted. It is preferred to create a database from the measurement results of the known effects of the components or the known conditions of a mouse model by using the same type of cells or animals so that changes in the gene expression levels are evaluated as a pattern. Such methods include those using a multivariate analysis, as described above.

Association with the detection results of a gene and functions (effects) of a test substance can be carried out as follows. The gene detection results are compared with a control, and if a change is observed in a gene expression level only when a subject organism has contacted, ingested or been administered the test substance, the test substance is determined to have been effective on the gene and to have had inhibitory or restorative effects on the condition of an eye disease.

Alternatively, an MT method as a multivariate analysis is carried out as follows: the control data are set as a base space, and the data from a subject organism affected by an eye disease are set as a signal space, and the distance with the base space is shown by the Mahalanobis distance to determine the value (the distance set as a determination base). Then, the data from a subject organism that has contacted, ingested or been administered a test substance is set as another signal space and the distance from the base space is shown by the Mahalanobis distance. When it is below the determination value described above, the test substance is determined to have inhibitory or restorative effects on the condition of the eye disease (reference: Basic Offline Quality Engineering, Genichi Taguchi, Yoshiko Yokoyama, Japanese Standards Association).

For such determination, the determination formula described above in “7-5 Evaluation of Conditions of Eye Disease” may be used.

By using the method related to the present invention, a substance (food, drugs, etc.) having inhibitory or restorative functions on the condition of an eye disease may be screened.

In the following, examples of the present invention are described in detail. However, the present invention is not limited to those examples.

### Example 1

### 1. Nucleic-Acid Microarray

Example 1 was conducted by using a nucleic-acid microarray (Genopal™, Mitsubishi Rayon Co., Ltd.) that mounts the probes described in Table 1 (SEQ ID Nos.: 1254).

### 2. Mouse Model with Eye Disease

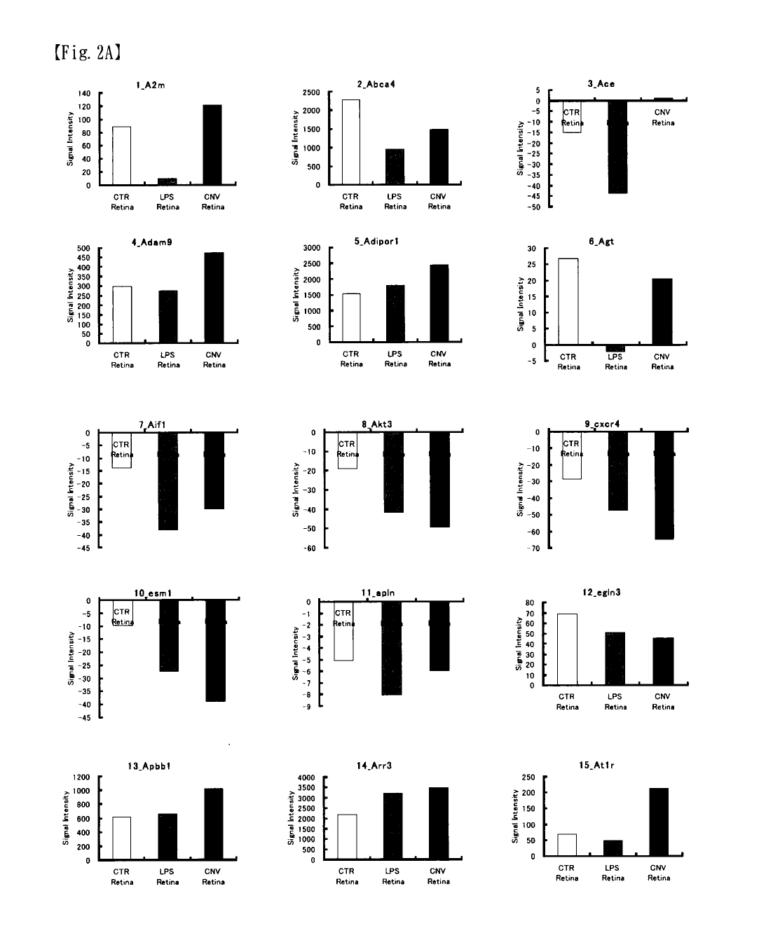

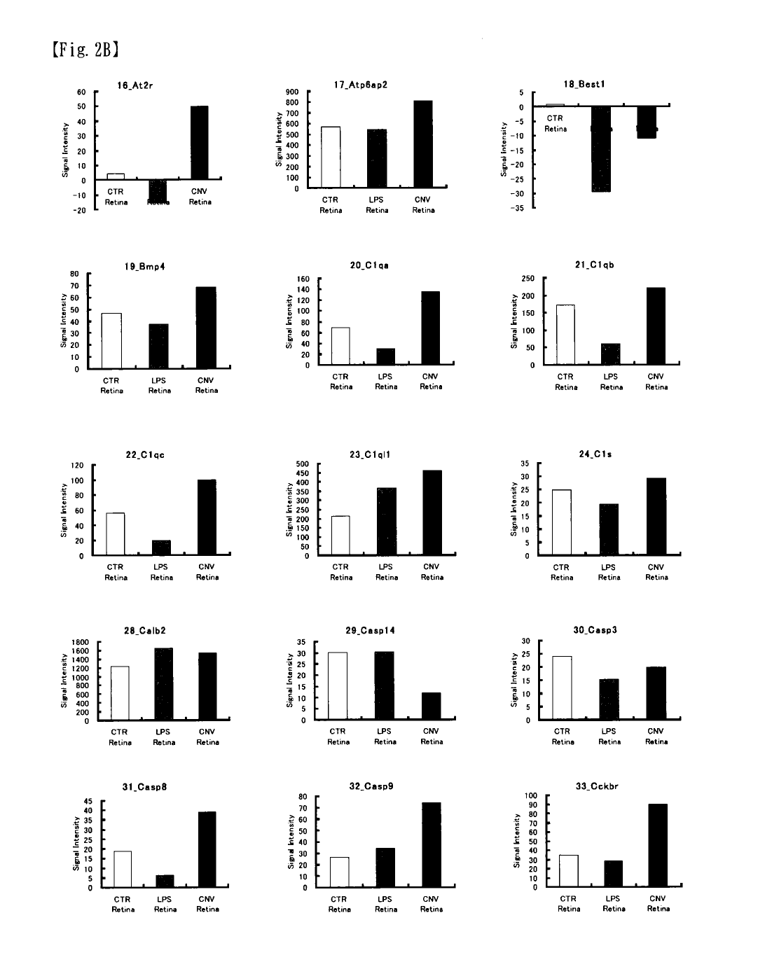

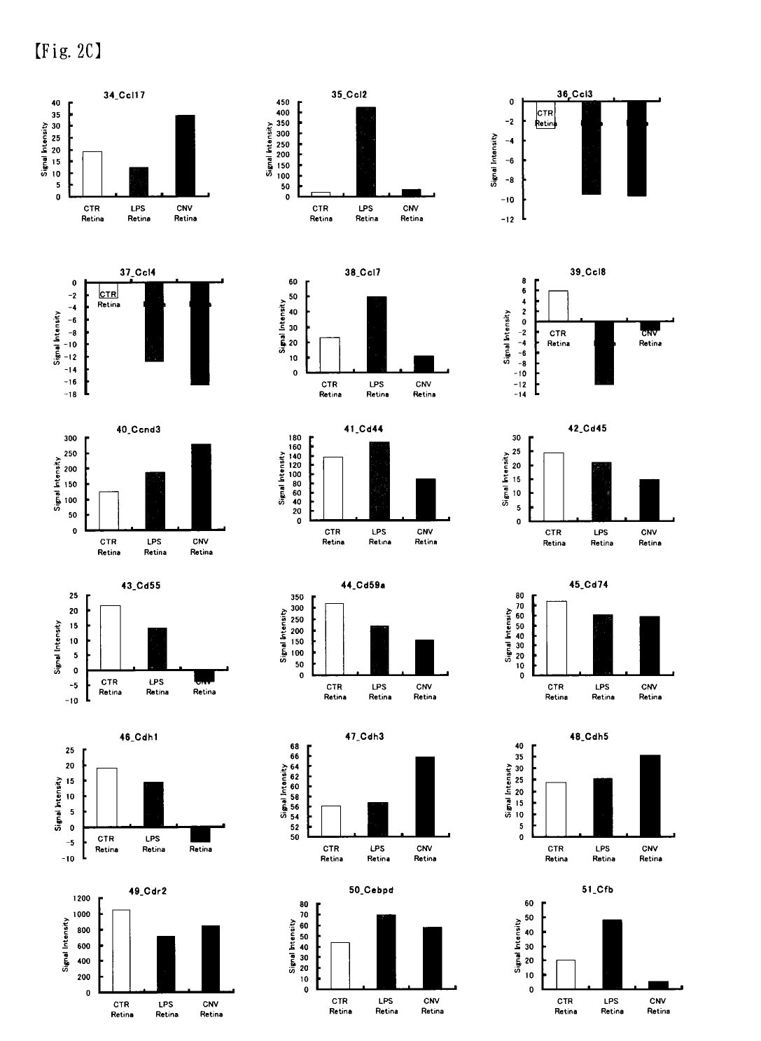

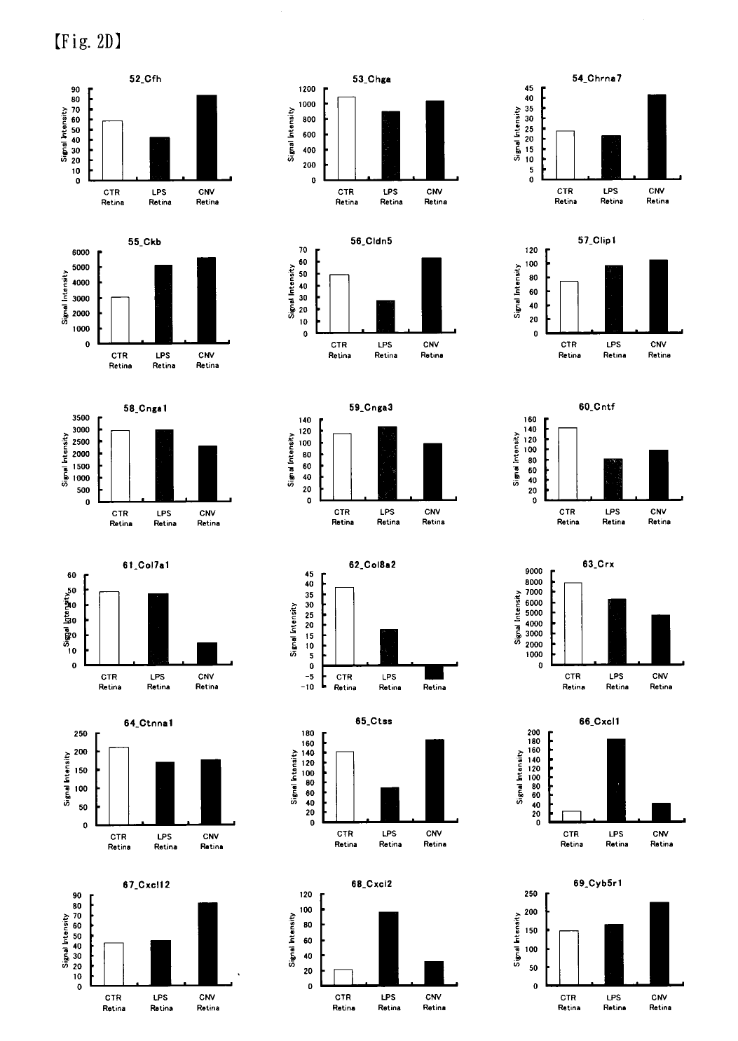

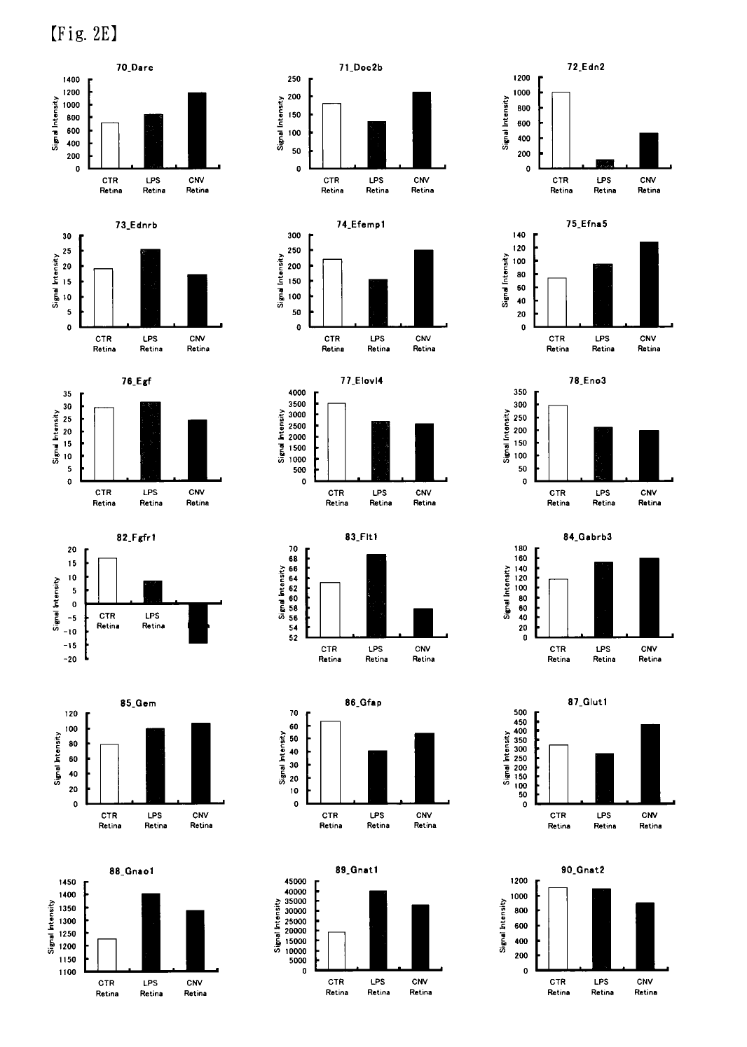

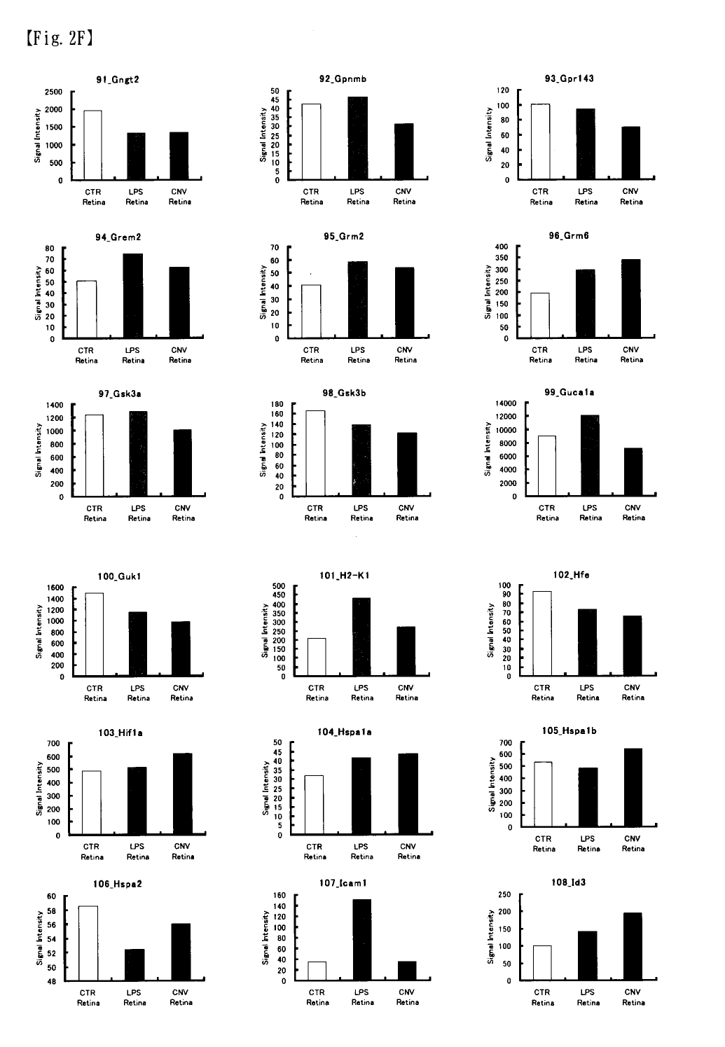

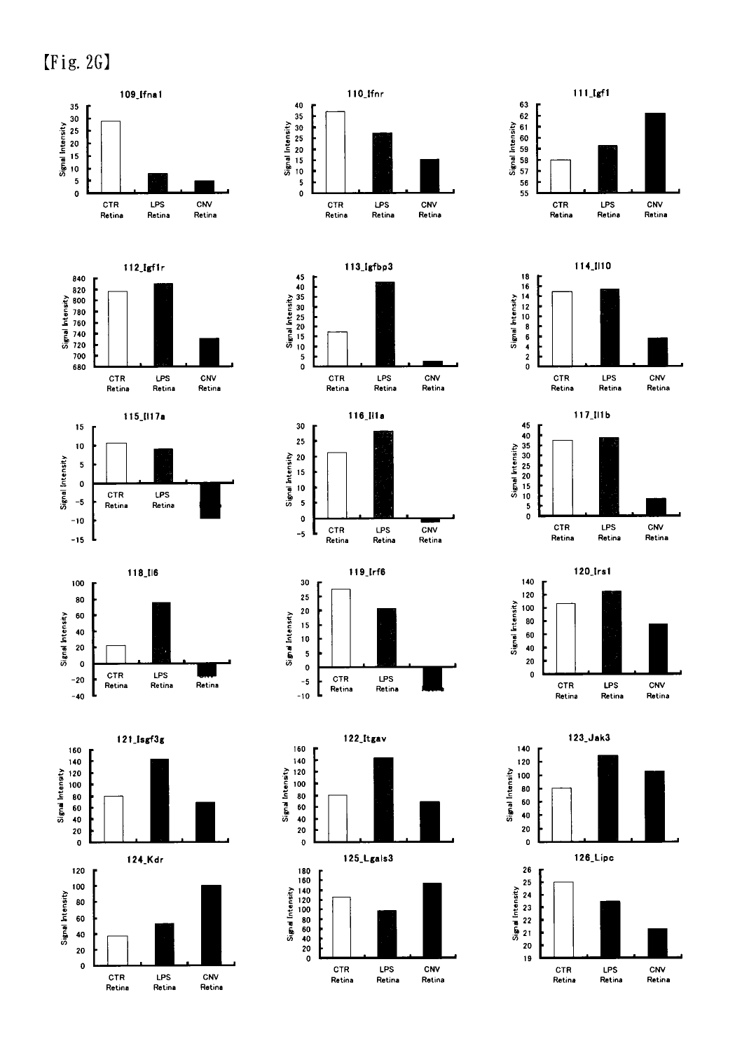

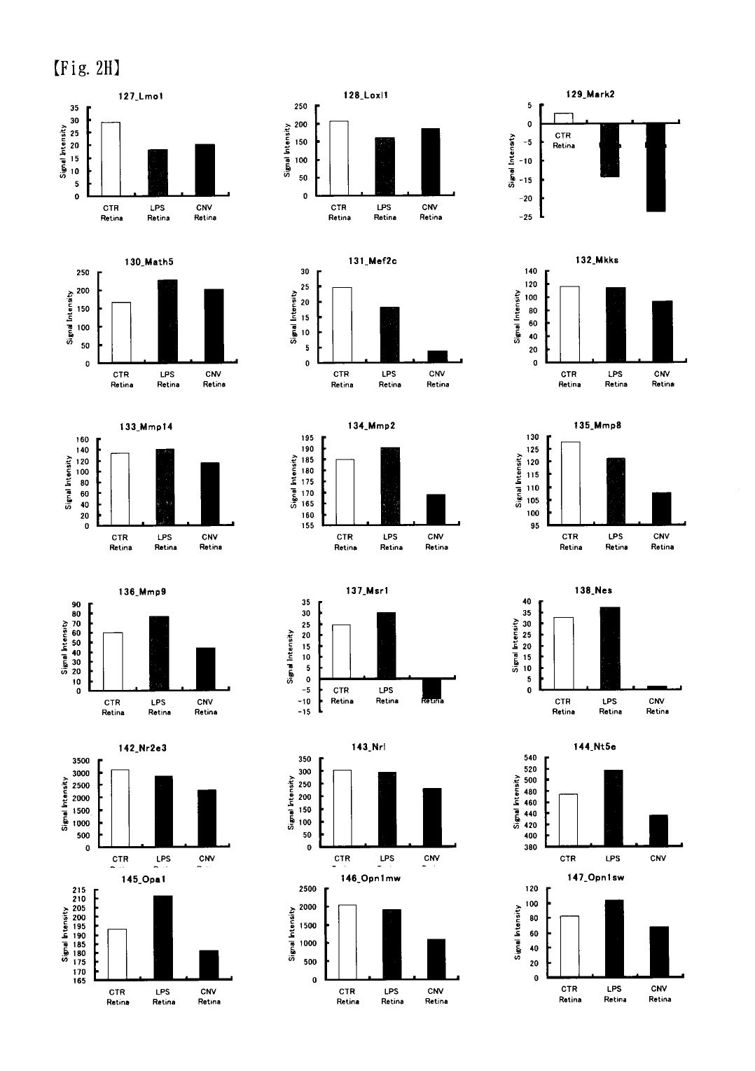

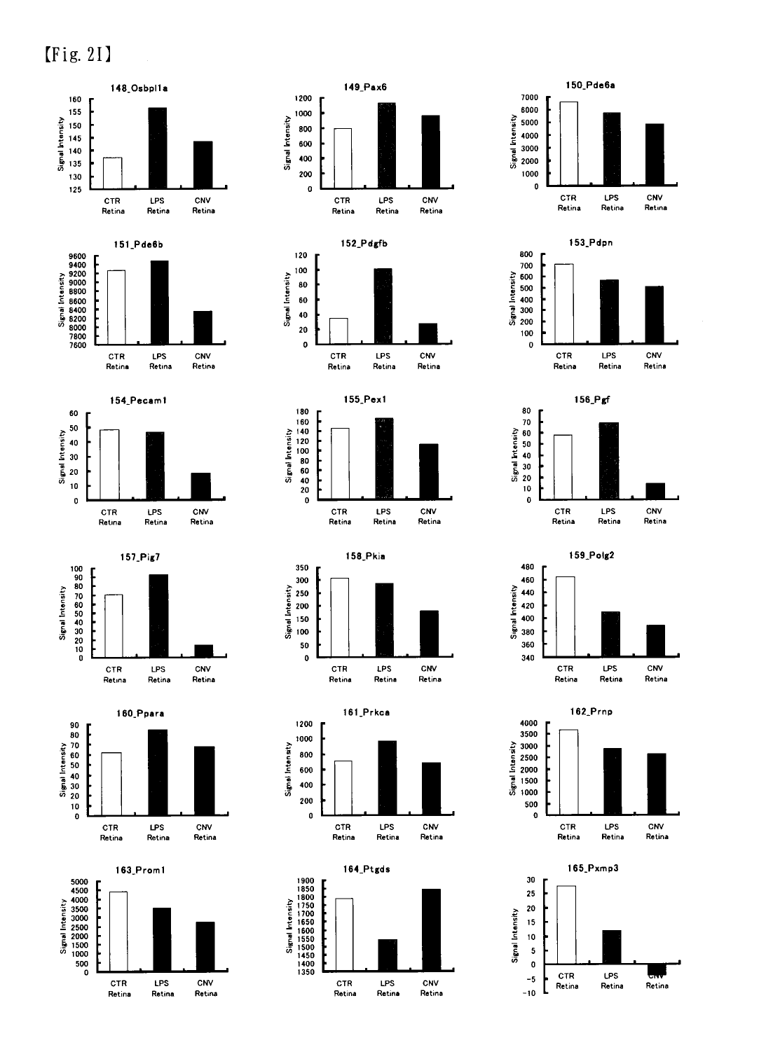

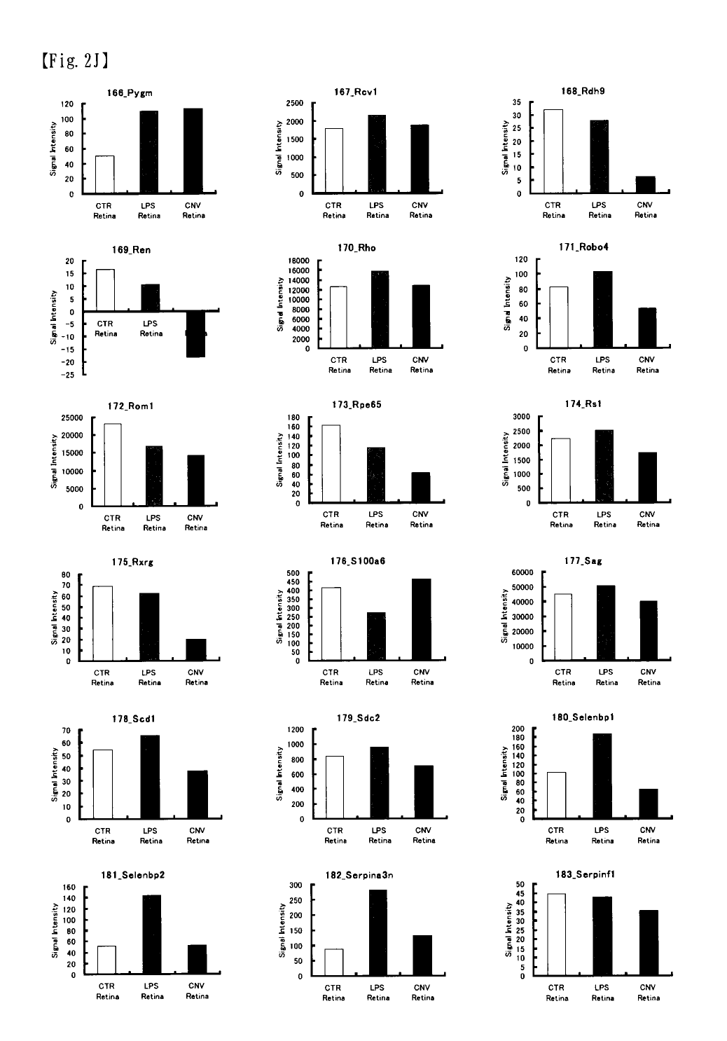

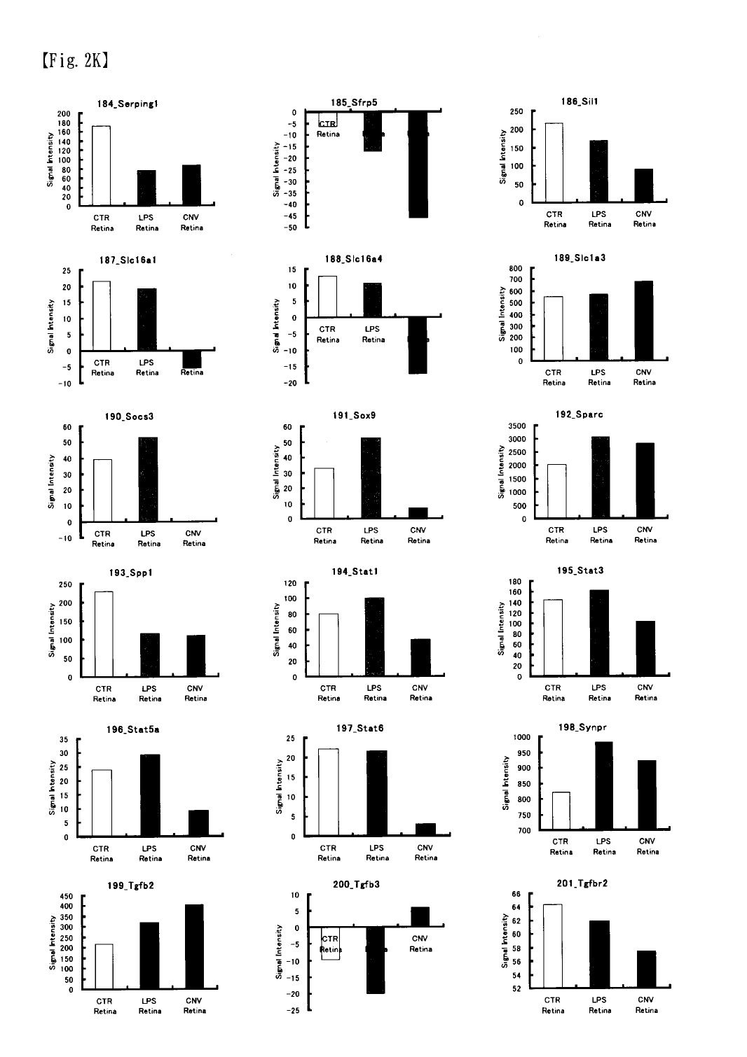

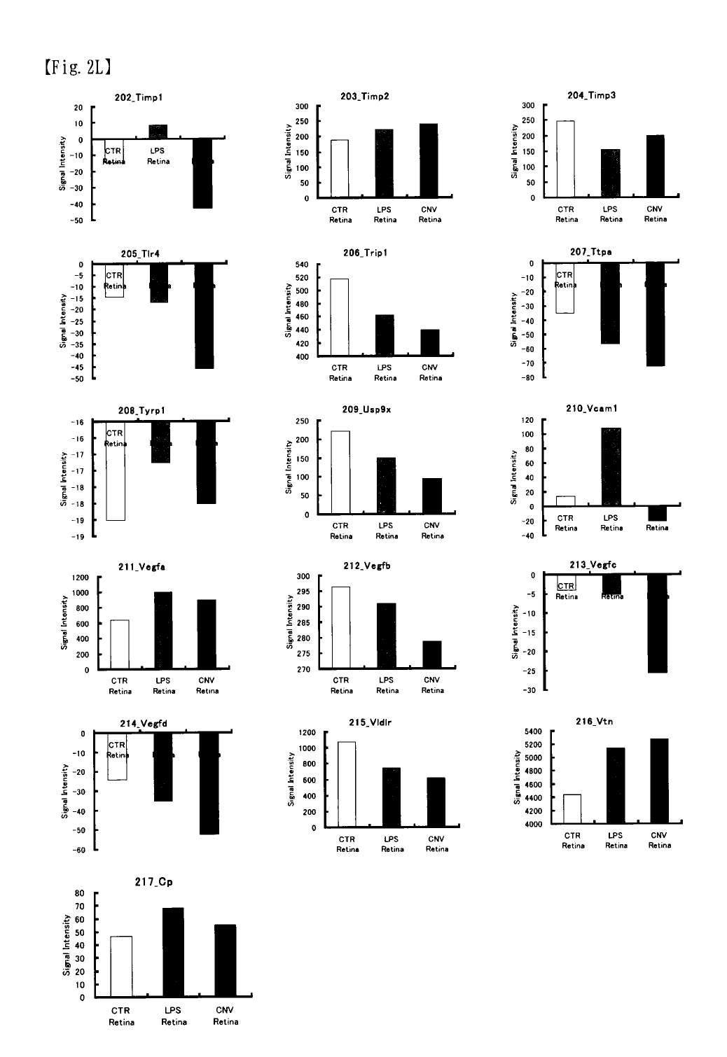

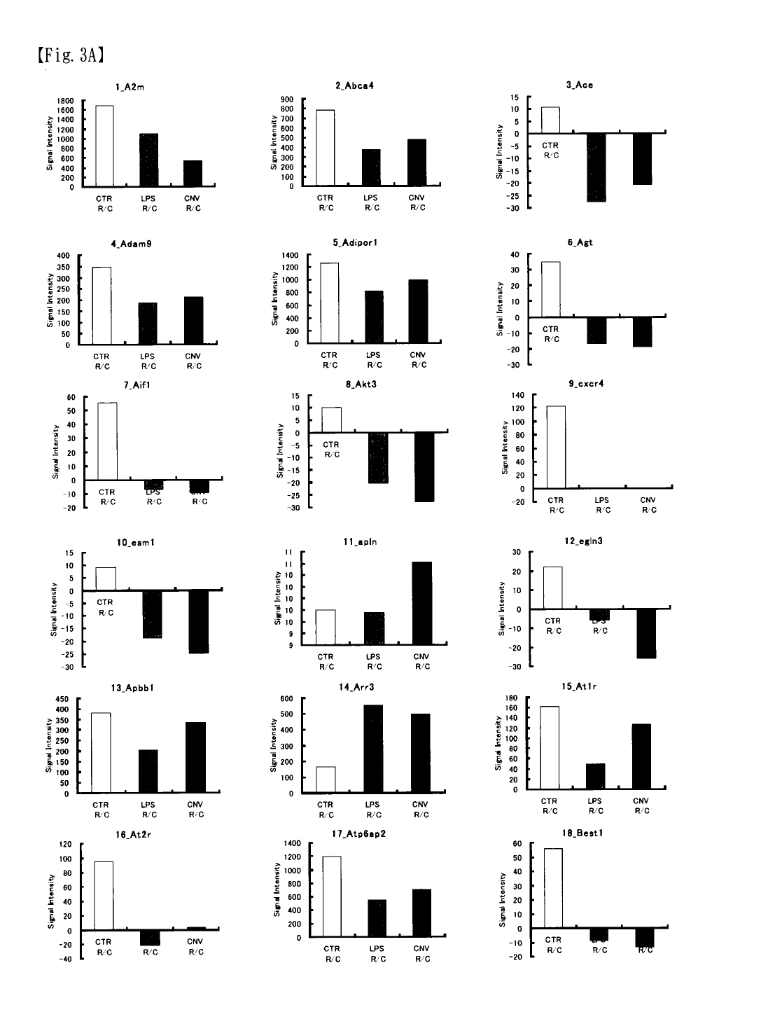

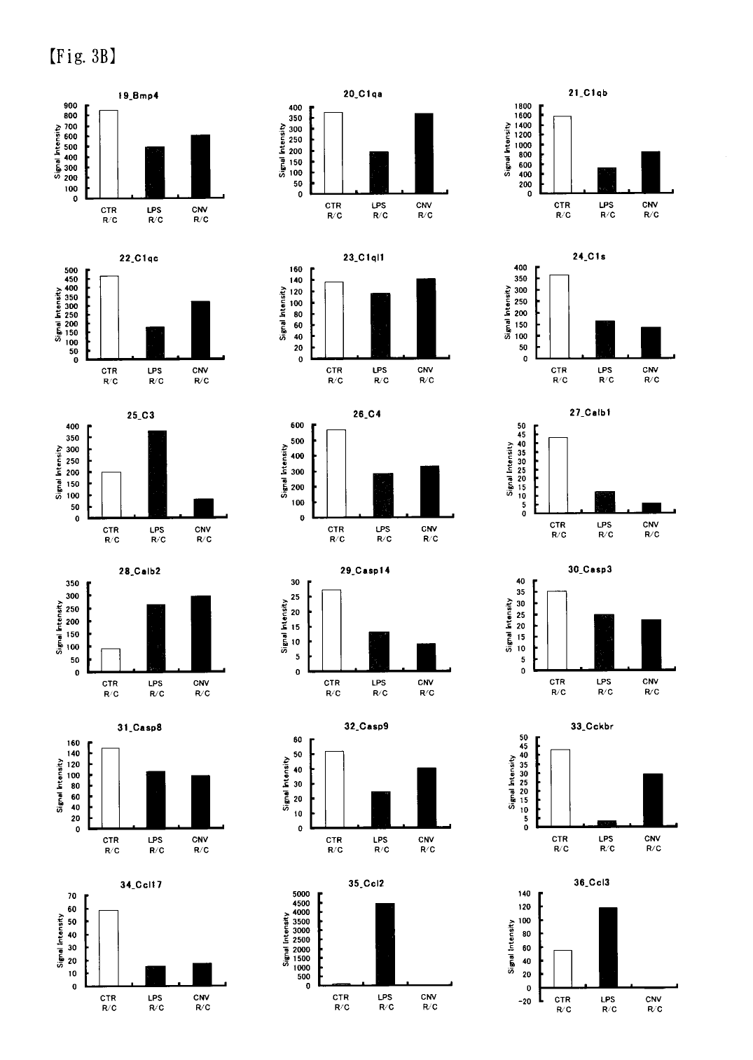

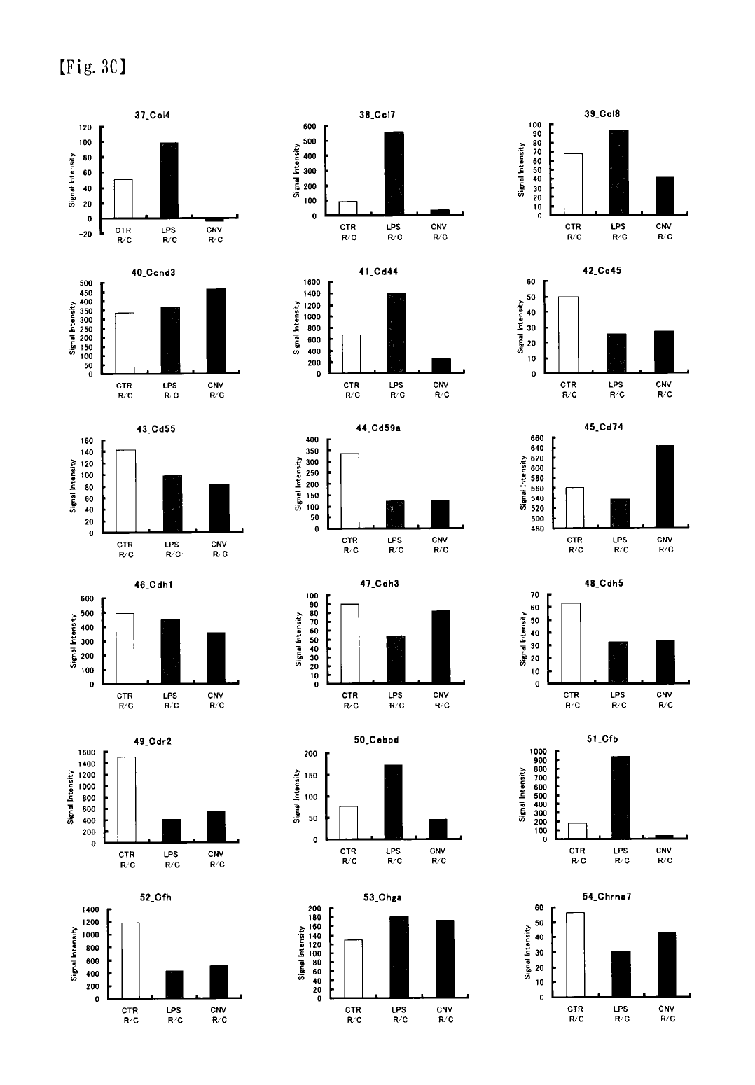

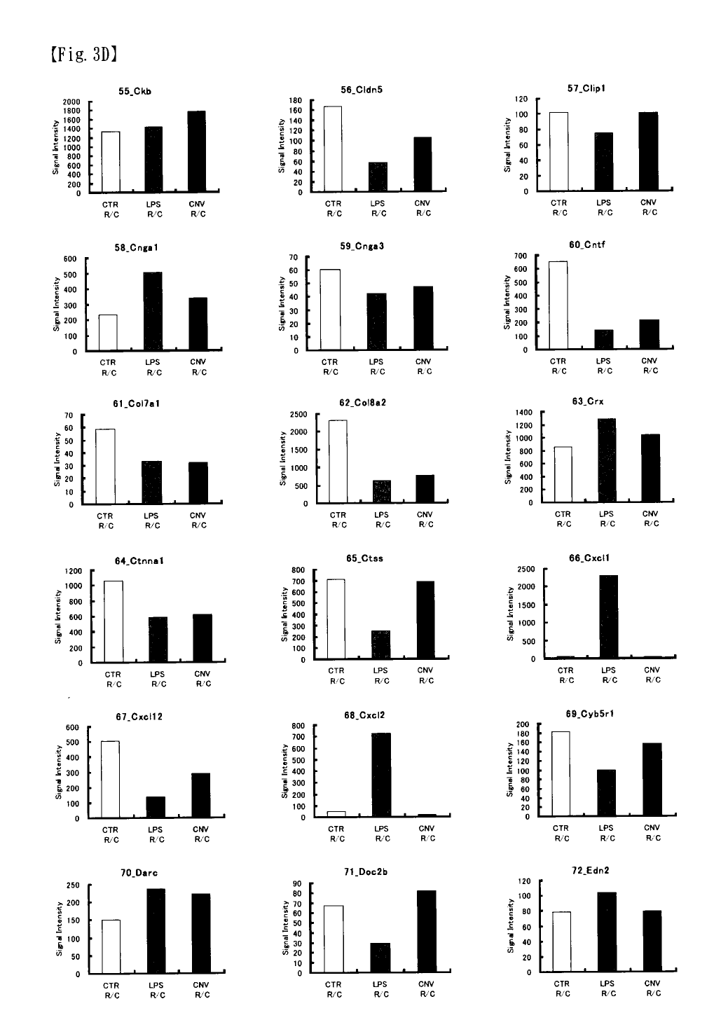

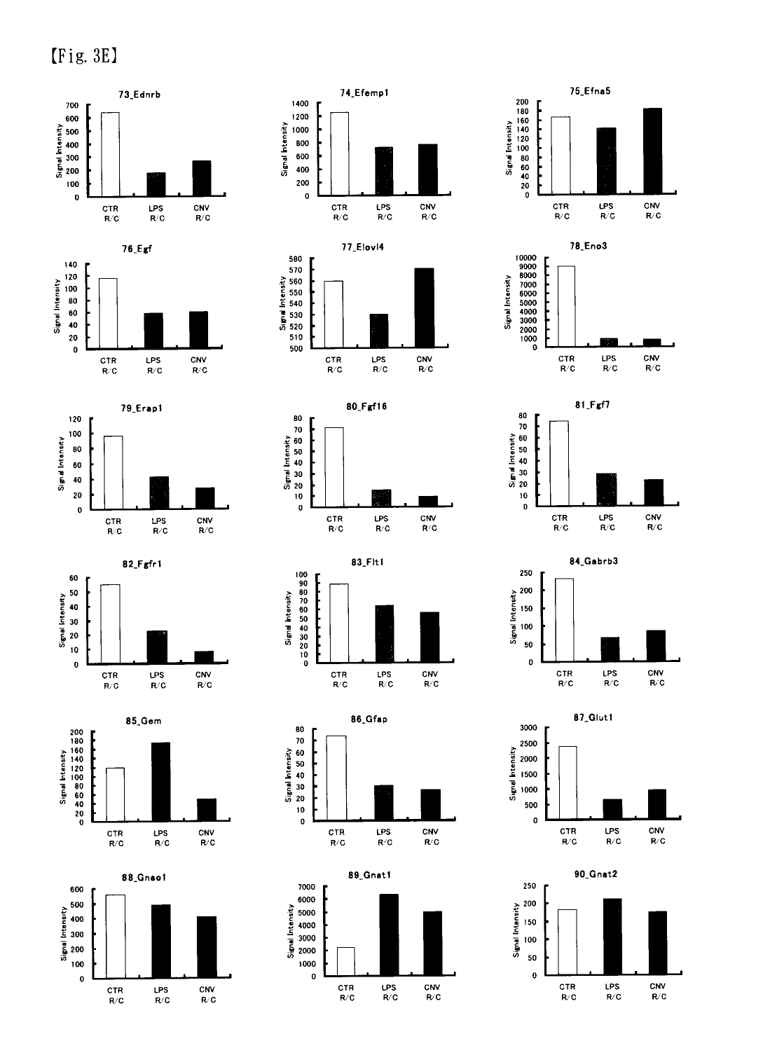

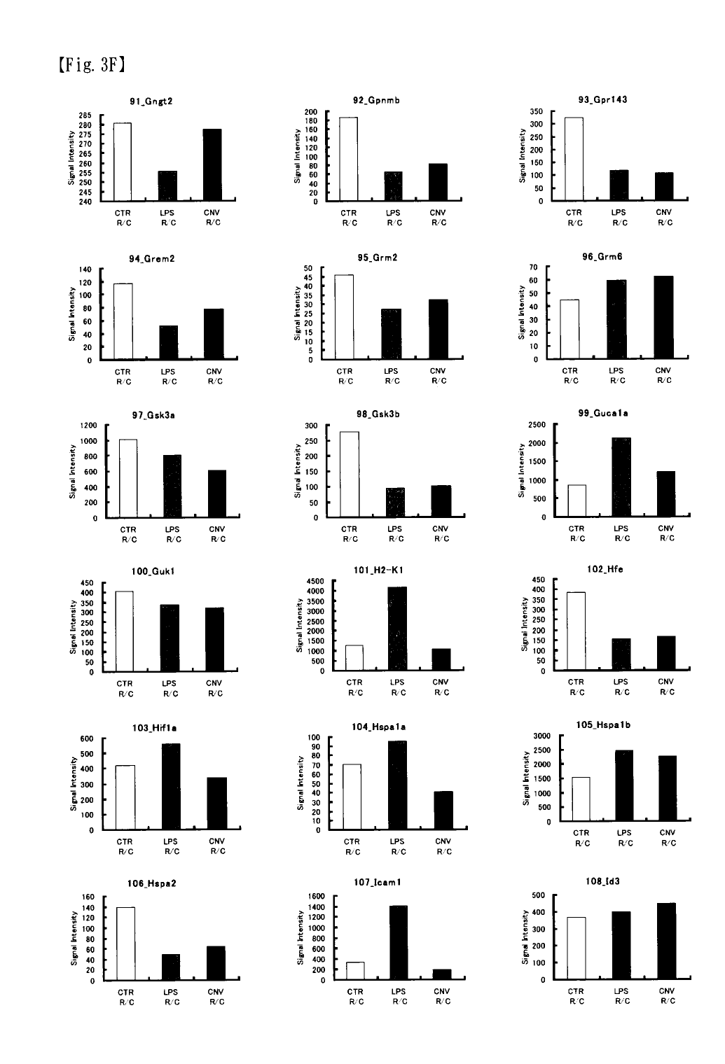

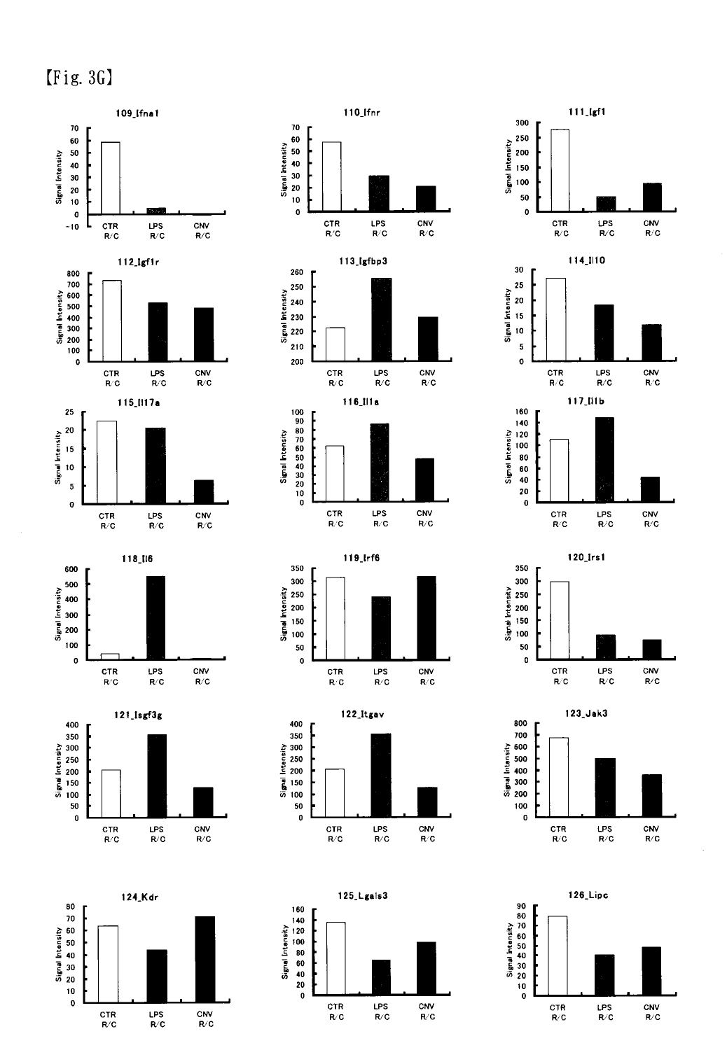

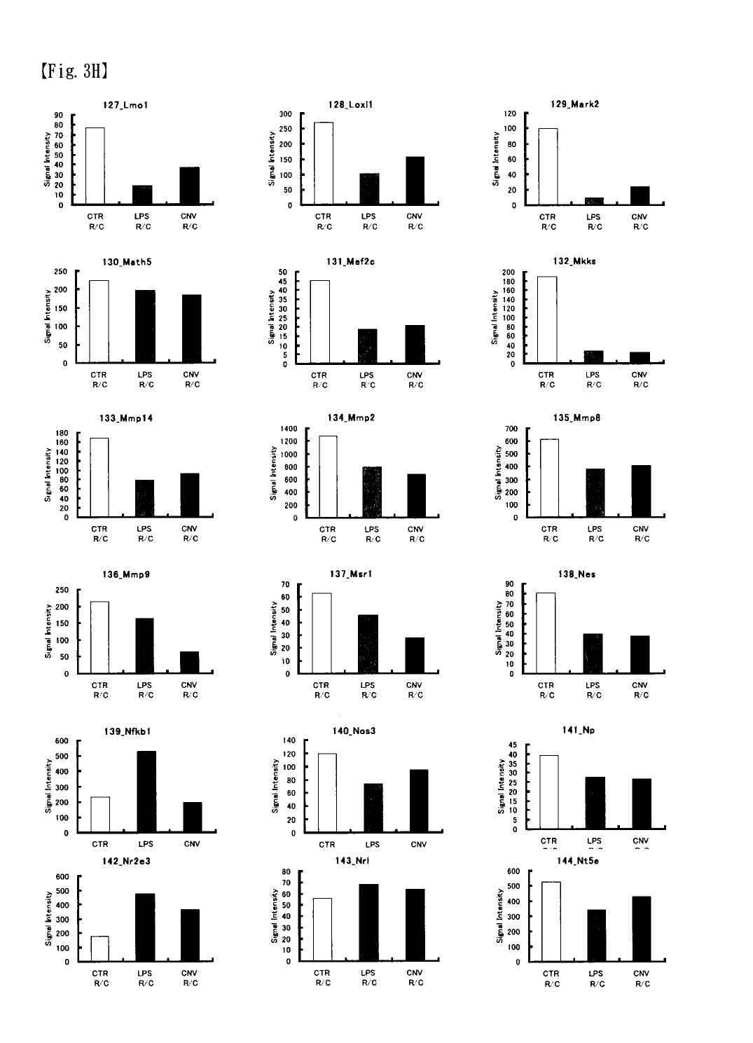

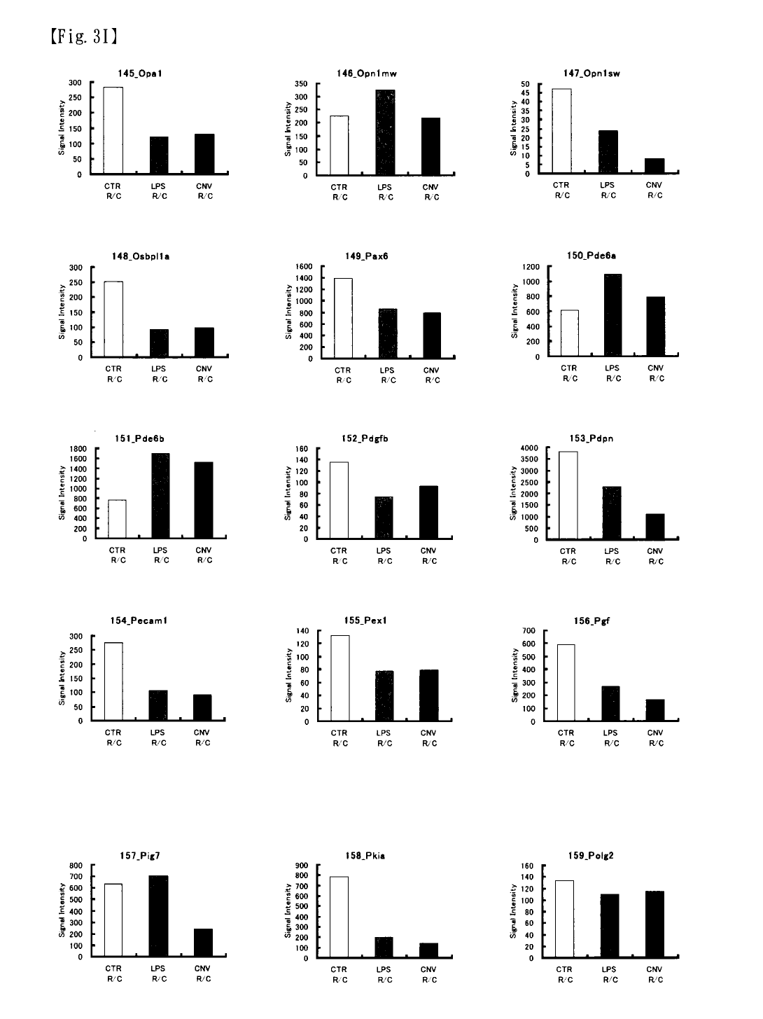

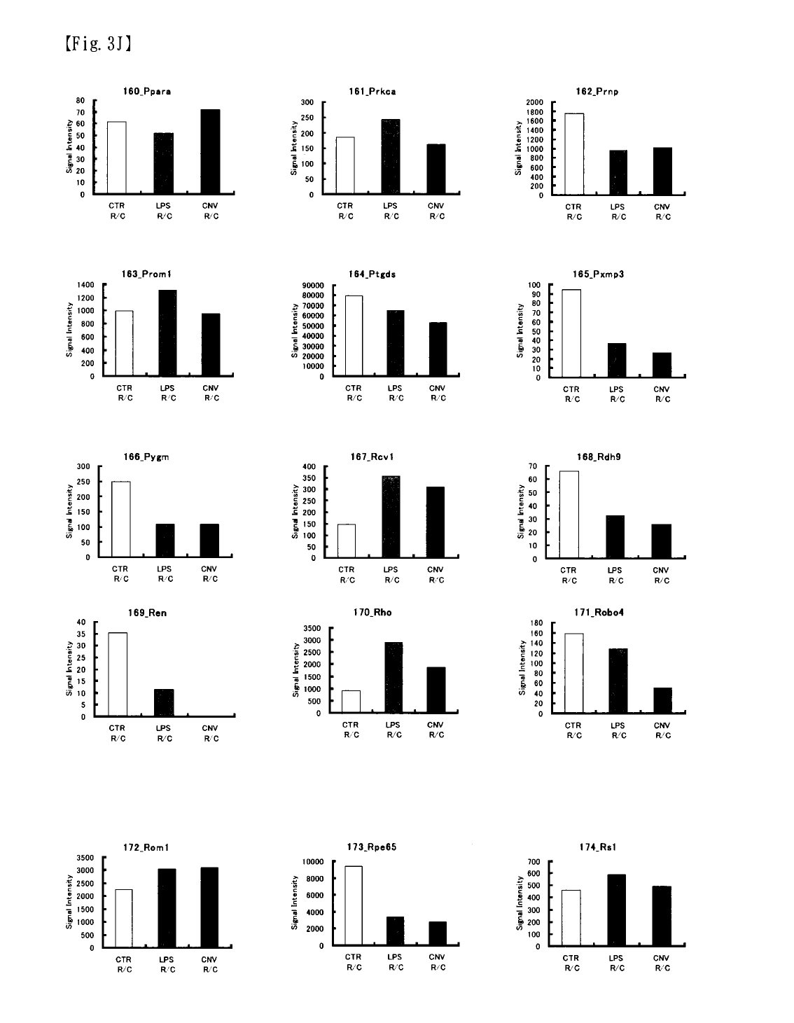

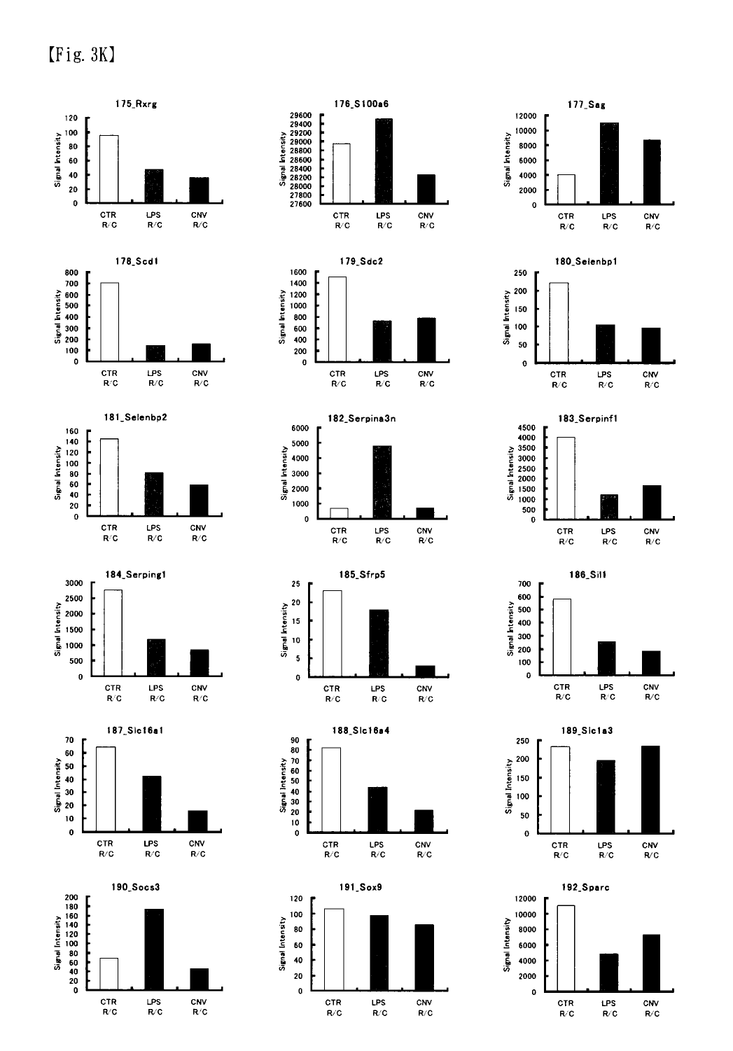

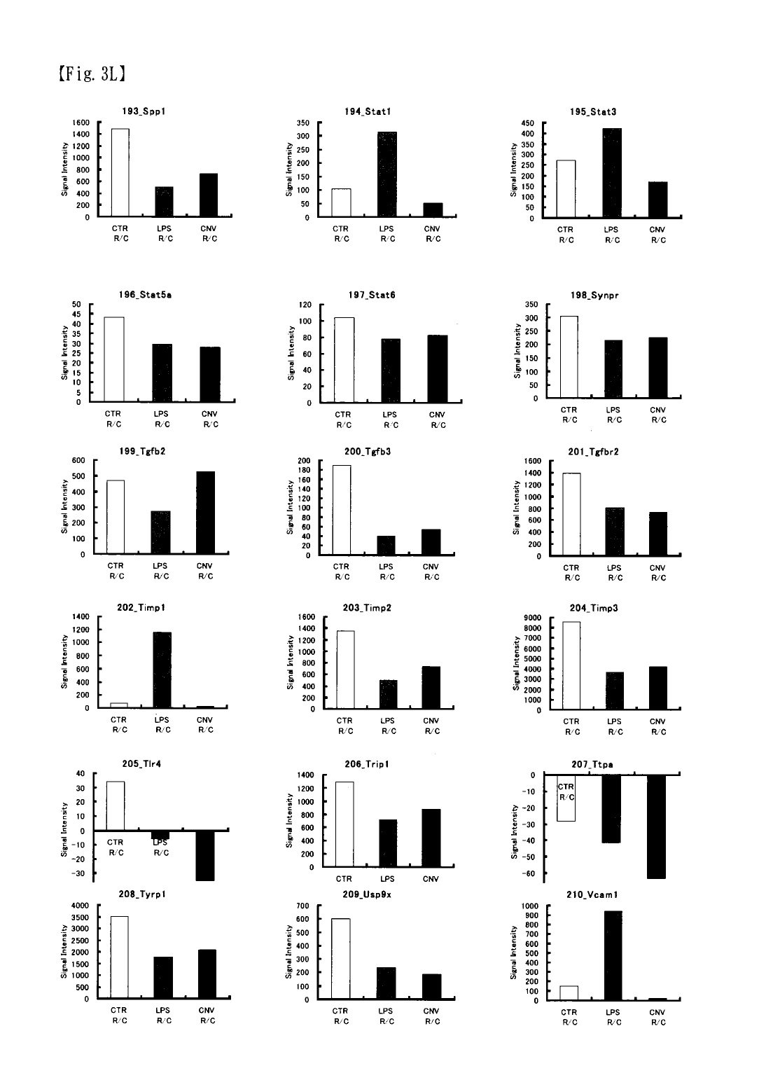

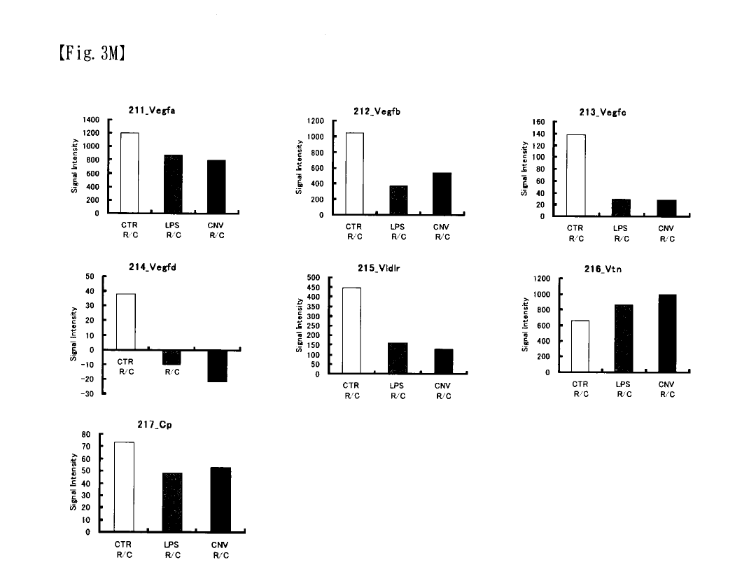

As examples of a mouse model with an eye disease, the present example uses the following: a mouse with choroidal neovascularization caused by irradiating a laser to induce angiogenesis (CNV mouse); and a mouse with inflammation induced using lipopolysaccharide (LPS) as stimulus (LPS mouse). Information pertaining to each mouse is shown in Table 2 below. The “ID term” column of Table 2 shows the same as those in FIGS. 2 and 3 and Table 3.

TABLE 2
animal

| ID | | | |
|---|---|---|---|
| term | animal | age in weeks | condition |
| CTR | C57BL/6 mouse | 8-10 weeks | normal |
| LPS | C57BL/6 mouse | 8-10 weeks | LPS stimulus: 6 hrs after |
| | | | intraperitoneal |
| | | | administration of |
| | | | LPS 0.2 mg (PBS (—) solution) |
| CNV | C57BL/6 mouse | 8-10 weeks | CNV model: 1 week after |
| | | | inducing angiogenesis by 10 μs |
| | | | irradiation of laser |
| | | | (wavelength 532 nm, |
| | | | output 100 mW) |

site

| ID term | site |
|---|---|
| Retina | retina |
| R/C | retinal pigment epithelium/choroid |

### 3. Preparation of Sample Solution

Total RNA was extracted from the retina and pigment epithelium/choroid samples taken from the mice model above. Table 3 shows the measured sample groups.

TABLE 3
measurement sample (total 8 groups)

| sample group | ID term | content | # of samples |
|---|---|---|---|
| 1 | CTR_Retina | retina of normal mouse | 6 |
| 2 | CTR_R/C | retinal pigment | 6 |
| | | epithelium/choroid of | |
| | | normal mouse | |
| 3 | LPS_Retina | retina of LPS mouse | 5 |
| 4 | LPS_R/C | retinal pigment | 5 |
| | | epithelium/choroid of | |
| | | LPS mouse | |
| 5 | CNV_Retina | retina of CNV mouse | 5 |
| | | model | |
| 6 | LPS_R/C | retinal pigment | 5 |
| | | epithelium/choroid of | |
| | | CNV mouse model | |

Using a MessageAmp II-Biotin Enhanced aRNA Amplification Kit (Applied Biosystems), aRNA was prepared from 1 μg of the extracted total RNA according to the attached protocol. Then, 5 μg of the obtained aRNA was placed in a plastic tube, 4 μL of 5× Array Fragmentation Buffer included in MessageAmpII-Biotin Enhanced Kit (Applied Biosystems) was added and diluted to the total of 20 μL. The mixture was stirred well, and heated at 94° C. for 7.5 minutes for fragmentation. A sample solution to be reacted with Genopal was prepared by mixing 24 μL of 1M Tris-HCl solution (Invitrogen), 24 μL of 1M NaCl solution (nacalai tesque) and 20 μL of 0.5% Tween 20 solution, respectively, and diluted to 180 μL by using Nuclease-free water. Then, 20 μL of the solution after fragmentation was mixed to prepare a sample solution.

### 3. Measurement

The nucleic-acid microarray (hereinafter also referred to as a “DNA chip”) was immersed in the sample solution, and hybridization reactions were conducted at 65° C. for 16 hours. After removing the sample solution used for the hybridization from the DNA chip, the DNA chip was immersed in a 65° C. 0.12M TNT solution (0.12M Tris-HCl, 0.12M NaCl, 0.5% Tween 20 solution) (for 20 minutes 2 times), then immersed in a 0.12M TN solution (0.12M Tris-HCl, 0.12M NaCl) heated to 65° C. for 10 minutes, and washed. To detect a signal in the DNA chip, a DNA chip detector device (MB-M3A, manufactured by Yokogawa Electric Corp.: laser wavelength: 633 nm) was used to measure the fluorescence intensity of Cy5 (exposure time: 0.1 sec., 1 sec., 4 sec., 40 sec.).

### 4. Data Analysis

Data were analyzed as follows:

(i) subtract the average value of the background;
(ii) correct the values by using a positive control gene (β-actin, GAPDH, RPLP0);
(iii) calculate the mean value, the expression ratio and the P value after correction (expression ratio is shown based on the CTR).
(iv) pick a gene with a significant change (a P value of 0.05 or less); and
(v) create a determination formula by using a bilateral T method which is one of the MT methods used to assign gene weights.

### 5. Results

Measurement results of a retina site (Retina) are shown in FIG. 2A-L, and measurement results of the retinal pigment epithelium and choroid site (R/C) are shown in FIG. 3A-M. Also, genes that show significant variations in expression levels in each measurement sample are shown in Table 4 below. Table 4 shows gene clusters that show changes in expression levels in CNV mice model and gene clusters that show changes in expression levels in LPS mice, respectively. Genes are listed in Table 5 in the order from a gene that showed greater contribution to a gene that showed less contribution to the calculation with respect to a normal mouse.

TABLE 4-1
gene clusters retina

| | |
|---|---|
| group i | group ii |
| significant change | significant change |
| in CNV | in LPS |

| probe # | symbol | probe # | symbol |
|---|---|---|---|
| 97 | Gsk3a | 101 | H2-K1 |
| 93 | Gpr143 | 118 | Il6 |
| 173 | Rpe65 | 66 | Cxcl1 |
| 102 | Hfe | 102 | Hfe |
| 206 | Trip1 | 206 | Trip1 |
| 78 | Eno3 | 78 | Eno3 |
| 166 | Gygm | 166 | Pygm |
| 85 | Gem | 171 | Robo4 |
| 123 | Jak3 | 85 | Gem |
| 143 | Nrl | 123 | Jak3 |
| 13 | Apbb1 | 87 | Glut1 |
| 2 | Abca4 | 2 | Abca4 |
| 120 | Irs1 | 120 | Irs1 |
| 175 | Rxrg | 40 | Ccnd3 |
| 40 | Ccnd3 | 139 | Nfkb1 |
| 209 | Usp9x | 56 | Cldn5 |
| 215 | Vldir | 209 | Usp9x |
| 17 | Atp6ap2 | 215 | Vldlr |
| 136 | Mmp9 | 159 | Pax6 |
| 238 | Jun | 238 | Jun |
| 186 | Sil1 | 186 | Sil1 |
| 44 | Cd59a | 49 | Cdr2 |
| 49 | Cdr2 | 3 | Ace |
| 154 | Pecam1 | 5 | Adipor1 |
| 158 | Pkia | 7 | Aif1 |
| 157 | Pig7 | 223 | Apln |
| 156 | Pgf | 14 | Arr3 |
| 4 | Adam9 | 15 | At1r |
| 5 | Adipor1 | 18 | Best1 |
| 7 | Aif1 | 23 | C1ql1 |
| 8 | Akt3 | 47 | Cdh3 |
| 230 | Aoc3 | 55 | Ckb |
| 14 | Arr3 | 57 | Clip1 |
| 15 | At1r | 217 | Cp |
| 16 | At2r | 63 | Crx |
| 18 | Best1 | 221 | Cxcr4 |
| 23 | C1gl1 | 73 | Ednrb |
| 32 | Casp9 | 75 | Efna5 |
| 33 | Cckbr | 12 | Egln3 |
| 47 | Cdh3 | 77 | Elovl4 |
| 55 | Ckb | 242 | Epo |
| 57 | Clip1 | 254 | Furin |
| 58 | Cnga1 | 89 | Gnat1 |
| 61 | Col7a1 | 91 | Gngt2 |
| 225 | Cp | 95 | Grm2 |
| 63 | Crx | 98 | Gsk3b |
| 67 | Cxcl12 | 100 | Guk1 |
| 69 | Cyb5r1 | 108 | Id3 |
| 70 | Darc | 122 | Itgav |
| 73 | Ednrb | 124 | Kdr |
| 75 | Efna5 | 237 | Mapk3 |
| 12 | Egln3 | 129 | Mark2 |
| 77 | Elovl4 | 134 | Mmp2 |
| 242 | Epo | 147 | Opn1sw |
| 83 | Flt1 | 155 | Pex1 |
| 254 | Furin | 160 | Ppara |
| 88 | Gnao1 | 161 | Prkca |
| 89 | Gnat1 | 162 | Pmp |
| 90 | Gnat2 | 163 | Prom1 |
| 91 | Gngt2 | 231 | Ptgs1 |
| 95 | Grm2 | 232 | Ptgs2 |
| 96 | Grm6 | 170 | Rho |
| 98 | Gsk3b | 172 | Rom1 |

retina pigment epithelium/choroid

| | |
|---|---|
| group iii | group iv |
| significant change | significant change |
| in CNV | in LPS |

| probe # | symbol | probe # | symbol |
|---|---|---|---|
| 105 | Hspa1b | 105 | Hspa1b |
| 97 | Gsk3a | 116 | Il1a |
| 117 | Il1b | 101 | H2-K1 |
| 107 | Icam1 | 117 | Il1b |
| 106 | Hspa2 | 107 | Isam1 |
| 111 | Igf1 | 118 | Il6 |
| 93 | Gpr143 | 106 | Hspa2 |
| 92 | Gpnmb | 111 | Igf1 |
| 173 | Rpe65 | 93 | Gpr143 |
| 165 | Pxmp3 | 66 | Cxcl1 |
| 102 | Hfe | 92 | Gpnmb |
| 203 | Timp2 | 173 | Rpe65 |
| 82 | Fgfr1 | 165 | Pxmp3 |
| 79 | Erap1 | 102 | Hfe |
| 78 | Eno3 | 193 | Spp1 |
| 166 | Pygm | 65 | Ctss |
| 62 | Col8a2 | 203 | Timp2 |
| 171 | Robo4 | 82 | Fgfr1 |
| 51 | Cfb | 121 | Isgf3g |
| 85 | Gem | 79 | Erap1 |
| 131 | Mef2c | 78 | Eno3 |
| 194 | Stat1 | 68 | Cxcl2 |
| 205 | Tlr4 | 166 | Pygm |
| 21 | C1qb | 62 | Cola2 |
| 195 | Stat3 | 51 | Cfb |
| 87 | Glut1 | 190 | Socs3 |
| 38 | Ccl7 | 131 | Mef2c |
| 120 | Irs1 | 194 | Stat1 |
| 64 | Ctnna1 | 226 | Tnfa |
| 148 | Osbpl1a | 205 | Tlr4 |
| 128 | Loxl1 | 21 | C1qb |
| 175 | Rxrg | 13 | Apbb1 |
| 204 | Timp3 | 195 | Stat3 |
| 40 | Ccnd3 | 87 | Glut1 |
| 212 | Vegfb | 38 | Ccl7 |
| 210 | Vcam1 | 2 | Abca4 |
| 213 | Vegfc | 120 | Irs1 |
| 60 | Cntf | 64 | Ctnna1 |
| 56 | Cldn5 | 148 | Osbpl1a |
| 241 | Ctgf | 128 | Loxl1 |
| 209 | Usp9x | 175 | Rxrg |
| 215 | Vldlr | 204 | Timp3 |
| 200 | Tgfb3 | 202 | Timp1 |
| 138 | Nes | 40 | Ccnd3 |
| 133 | Mmp14 | 212 | Vegfb |
| 136 | Mmp9 | 210 | Vcam1 |
| 25 | C3 | 213 | Vegfc |
| 164 | Ptgds | 60 | Cntf |
| 153 | Pdpn | 139 | Nfkb1 |
| 233 | Pla2g5 | 56 | Cldn5 |
| 127 | Lmo1 | 241 | Ctgf |
| 222 | Esm1 | 209 | Usp9x |
| 50 | Cebpd | 215 | Vldlr |
| 238 | Jun | 200 | Tgfb3 |
| 187 | Slc16a1 | 149 | Pax6 |
| 186 | Sil1 | 17 | Atp6ap2 |
| 178 | Scd1 | 138 | Nes |
| 35 | Ccl2 | 133 | Mmp14 |
| 234 | Il6st | 36 | Ccl3 |
| 41 | Cd44 | 22 | C1qc |
| 44 | Cd59a | 153 | Pdpn |
| 183 | Serpinf1 | 233 | Pla2g5 |
| 49 | Cdr2 | 244 | Angpt-1 |

TABLE 4-2
gene clusters retina

| | |
|---|---|
| group i | group ii |
| significant change | significant change |
| in CNV | in LPS |

| probe # | symbol | probe # | symbol |
|---|---|---|---|
| 100 | Guk1 | 179 | Sdc2 |
| 103 | Hif1a | 180 | Selenbp1 |
| 108 | Id3 | 181 | Selenbp2 |
| 109 | Ifna1 | 185 | Sfrp5 |
| 110 | Ifnr | 191 | Sox9 |
| 112 | Igf1r | 192 | Sparc |
| 122 | Itgav | 199 | Tgfb2 |
| 124 | Kdr | 211 | Vegfa |
| 134 | Mmp2 | | |
| 142 | Nr2e3 | | |
| 145 | Opa1 | | |
| 146 | Opn1mw | | |
| 147 | Opn1sw | | |
| 150 | Pde6a | | |
| 161 | Prkca | | |
| 162 | Prnp | | |
| 163 | Prom1 | | |
| 231 | Ptgs1 | | |
| 172 | Rom1 | | |
| 179 | Sdc2 | | |
| 180 | Selenbp1 | | |
| 191 | Sox9 | | |
| 192 | Sparc | | |
| 198 | Synpr | | |
| 199 | Tgfb2 | | |
| 208 | Tyrp1 | | |
| 211 | Vegfa | | |
| 214 | Vegfd | | |

retina pigment epithelium/choroid

| | |
|---|---|
| group iii | group iv |
| significant change | significant change |
| in CNV | in LPS |

| probe # | symbol | probe # | symbol |
|---|---|---|---|
| 154 | Pecam1 | 127 | Lmo1 |
| 158 | Pkia | 222 | Esm1 |
| 48 | Cdh5 | 50 | Cebpd |
| 28 | Calb2 | 187 | Slc16a1 |
| 249 | Adam19 | 186 | Sil1 |
| 24 | C1s | 178 | Scd1 |
| 248 | Adam17 | 37 | Ccl4 |
| 157 | Pig7 | 227 | Tgfb1 |
| 156 | Pgf | 35 | Ccl2 |
| 16 | At2r | 239 | Fos |
| 18 | Best1 | 234 | Il6st |
| 43 | Cd55 | 44 | Cd59a |
| 9 | Cxcr4 | 183 | Serpinf1 |
| 76 | Egf | 49 | Cdr2 |
| 81 | Fgf7 | 236 | Mapk1 |
| 83 | Flt1 | 154 | Pecam1 |
| 122 | Itgav | 158 | Pkia |
| 124 | Kdr | 48 | Cdh5 |
| 125 | Lgals3 | 249 | Adam19 |
| 134 | Mmp2 | 24 | C1s |
| 135 | Mmp8 | 248 | Adam17 |
| 180 | Selenbp1 | 229 | Tnfrsf1a |
| 181 | Selenbp2 | 250 | Mmp1 |
| 197 | Stat6 | 251 | Mmp7 |
| 214 | Vegfd | 252 | Selp |
| | | 156 | Pgf |
| | | 15 | At1r |
| | | 16 | At2r |
| | | 39 | Ccl8 |
| | | 54 | Chrna7 |
| | | 57 | Clip1 |
| | | 9 | Cxcr4 |
| | | 69 | Cyb5r1 |
| | | 76 | Egf |
| | | 81 | Fgf7 |
| | | 83 | Flt1 |
| | | 94 | Grem2 |
| | | 124 | Kdr |
| | | 125 | Lgals3 |
| | | 126 | Lipc |
| | | 134 | Mmp2 |
| | | 135 | Mmp8 |
| | | 140 | Nos3 |
| | | 152 | Pdgfb |
| | | 253 | Sele |
| | | 180 | Selenbp1 |
| | | 181 | Selenbp2 |
| | | 197 | Stat6 |

TABLE 5

| conribution | | |
|---|---|---|
| order to | | |
| formula | gene name | η |
| 1 | Hspa1b | 28.7609 |
| 2 | Il1a | 28.75654 |
| 3 | Gsk3a | 28.73853 |
| 4 | H2-K1 | 28.66572 |
| 5 | Il1b | 28.35073 |
| 6 | Icam1 | 28.17819 |
| 7 | Il6 | 28.16219 |
| 8 | Hspa2 | 28.10675 |
| 9 | Igf1 | 28.06117 |
| 10 | Gpr143 | 27.93883 |
| 11 | Cscl1 | 27.90516 |
| 12 | Gpnmb | 27.83096 |
| 13 | Rpe65 | 27.76219 |
| 14 | Pxmp3 | 27.6787 |
| 15 | Irf6 | 27.63083 |
| 16 | Hfe | 27.58361 |
| 17 | Spp1 | 27.42699 |
| 18 | Ctss | 27.42325 |
| 19 | Math5 | 27.40593 |
| 20 | Doc2b | 27.39983 |
| 21 | Timp2 | 27.33578 |
| 22 | Fgfr1 | 27.31464 |
| 23 | Isgf3g | 27.27673 |
| 24 | Erap1 | 27.25751 |
| 25 | Trip1 | 27.24695 |
| 26 | Eno3 | 27.22714 |
| 27 | Cxcl2 | 27.21546 |
| 28 | Pygm | 27.16802 |
| 29 | Col8a2 | 27.14725 |
| 30 | Lobo4 | 27.08653 |
| 31 | Cfb | 27.05906 |
| 32 | Socs3 | 27.05319 |
| 33 | Gem | 27.05211 |
| 34 | Mef2c | 27.0515 |
| 35 | Stat1 | 27.04586 |
| 36 | Tnfa | 27.03798 |
| 37 | Tlr4 | 27.03179 |
| 38 | Jak3 | 27.00856 |
| 39 | Np | 27.0025 |
| 40 | C1qb | 26.98939 |
| 41 | Gabrb3 | 26.98768 |
| 42 | Nrl | 26.95518 |
| 43 | Apbb1 | 26.93522 |
| 44 | Stat3 | 26.93438 |
| 45 | Glut1 | 26.91839 |
| 46 | Ccl7 | 26.91131 |
| 47 | Abca4 | 26.89867 |
| 48 | Irs1 | 26.86307 |
| 49 | Ctnna1 | 26.84505 |
| 50 | Osbpl1a | 26.75947 |
| 51 | Loxl1 | 26.74482 |
| 52 | Rxrg | 26.7149 |
| 53 | Timp3 | 26.67336 |
| 54 | Timp1 | 26.66398 |
| 55 | Ccnd3 | 26.65889 |
| 56 | Vegfb | 26.6176 |
| 57 | Vcam1 | 26.60984 |
| 58 | Vegfc | 26.60278 |
| 59 | Cntf | 26.5788 |
| 60 | Nfkb1 | 26.55388 |
| 61 | Nt5e | 26.54656 |
| 62 | Cnga3 | 26.53593 |
| 63 | Cldn5 | 26.53553 |
| 64 | Ctgf | 26.52942 |
| 65 | Msr1 | 26.50954 |
| 66 | Actb | 26.49759 |
| 67 | Usp9x | 26.49715 |
| 68 | Vldlr | 26.47908 |
| 69 | Tgfb3 | 26.4436 |
| 70 | Pax6 | 26.43603 |
| 71 | Atp6ap2 | 26.41118 |
| 72 | Nes | 26.33202 |
| 73 | Mmp14 | 26.30448 |
| 74 | Ccl3 | 26.29844 |
| 75 | Mmp9 | 26.29316 |
| 76 | Ttpa | 26.29071 |
| 77 | Rs1 | 26.16883 |
| 78 | C1qc | 26.16276 |
| 79 | Slc16a4 | 26.16204 |
| 80 | C3 | 26.13161 |
| 81 | Ptgds | 26.12598 |
| 82 | Pdon | 26.11834 |
| 83 | Pla2g5 | 26.11101 |
| 84 | Angpt-1 | 26.07373 |
| 85 | Lmo1 | 26.03846 |
| 86 | Esm1 | 25.95695 |
| 87 | Cebpd | 25.93072 |
| 88 | Jun | 25.88974 |
| 89 | Slc16a1 | 25.88433 |
| 90 | Sil1 | 25.87845 |
| 91 | Scd1 | 25.86244 |
| 92 | Ccl4 | 25.83053 |
| 93 | Tgfb1 | 25.72079 |
| 94 | Ccl2 | 25.67051 |
| 95 | Fos | 25.66059 |
| 96 | Il6st | 25.64978 |
| 97 | Cd44 | 25.61791 |
| 98 | Cd59a | 25.61382 |
| 99 | Serpinf1 | 25.57669 |
| 100 | Cdr2 | 25.57435 |
| 101 | Mapk1 | 25.57417 |
| 102 | Cd45 | 25.55897 |
| 103 | Pecam1 | 25.52968 |
| 104 | Pkia | 25.50032 |
| 105 | Cdh5 | 25.18971 |
| 106 | Calb2 | 24.99968 |
| 107 | Adam19 | 24.87039 |
| 108 | C1s | 24.84763 |
| 109 | Adam17 | 24.66254 |
| 110 | Tnfrsf1a | 24.56724 |
| 111 | Pig7 | 24.46434 |
| 112 | Mmp1 | 24.41771 |
| 113 | Mmp7 | 24.26331 |
| 114 | Selp | 23.96141 |
| 115 | Pgf | 23.87452 |

Moreover, the results of a study using the determination formula are shown below. In the table, η represents the signal/noise ratio of the determination formula (S/N); the greater the value, the more preferable it is.

TABLE 6

| | | | | | | determination | |
|---|---|---|---|---|---|---|---|
| | | | | | | formula S/N | |
| | | | | | | | retinal |
| | | | | | | | pigment |
| | | | | | | | epi- |
| | | | | | | | thelium/ |
| | | | | | | retina | choroid |
| name of genes | | | | | | η | η |
| Cxcl1 | At2r | At1r | Cfb | | | 6.088 | 6.635 |
| Il6 | Pig7 | Jak3 | Timp1 | Mmp9 | At2r | 1.647 | 1.560 |
| Selenbp2 | Pgf | Sil1 | Cxcl2 | Calb2 | Eno3 | 3.844 | −5.020 |
| Nfkb1 | Rxrg | Usp9x | Il6 | Robo4 | Vcam1 | 5.507 | 4.392 |
| Cxcl1 | At2r | Tgfb2 | Cxcl1 | Cfb | Fgf7 | 6.236 | 4.384 |
| Cxcl1 | At2r | Vegfa | Cxcl1 | Cfb | Fgf7 | 8.165 | 5.239 |
| Cxcl1 | At2r | Vegfa | Cxcl1 | Cfb | Vegfb | 6.489 | 4.090 |
| Cxcl1 | At2r | Vegfa | Cxcl1 | Cfb | Vegfc | 7.277 | 5.030 |
| Il6 | Pig7 | Vegfa | Timp1 | Mmp9 | Vegfc | 4.588 | 19.102 |
| Cxcl1 | At2r | Vegfa | Cxcl1 | Cfb | Vegfd | 7.277 | 3.696 |
| Cxcl1 | At2r | Vegfa | Cxcl1 | Cfb | Vldlr | 6.487 | 2.478 |

From those results, to determine the condition of the retina, namely, to determine whether the condition resulted from light damage or from inflammation caused by an inflammation-inducing substance, it was shown that such conditions are preferred to be determined by focusing on genes Cxcl1, At2r, Vegfa, Cxcl1, Cfb and Fgf7. Also, to determine the condition in the retinal epithelial cells and choroid, namely, to determine whether the condition resulted from light damage or from inflammation caused by an inflammation-inducing substance, it was shown that such conditions are preferred to be determined by focusing on genes Il6, Pig7, Vegfa, Timp1, Mmp9 and Vegfc.

The results above have identified genes that show variations when a disorder caused by light, inflammation or angiogenesis occurs in the retina and retinal pigment epithelium/choroid of the eye. Also, genes that vary in the case of age-related macular degeneration have been also identified. By analyzing variations in the expression levels of these genes in the samples taken from a subject organism, the conditions of eye diseases in the subject organism, especially age-related macular degeneration, are evaluated. Also, from a sample taken from a subject organism that has contacted, ingested or been administered a test substance, the inhibitory or restorative functions of the test substance on the eye disease of the subject organism are evaluated by analyzing the change in the expression levels of those genes.

Namely, it was found that the method related to the present invention is capable of conducting objective evaluation of eye diseases, especially age-related macular degeneration, in a subject organism. Also, it was found that the method is capable of evaluating inhibitory or restorative effects of a test substance on eye diseases, especially on age-related macular degeneration

### DESCRIPTION OF NUMERICAL REFERENCES

11 through hole
21 porous plate
31 hollow fiber
41 plate material

### SEQUENCE TEXT

SEQ ID Nos. 1˜255 synthetic DNA

1. A method for evaluating the condition of an eye disease, comprising:
measuring the expression variation of a gene with a microarray, wherein the gene is classified as at least one selected from the group consisting of:
(a) a gene associated with both light damage to the retina and retinal inflammation caused by an inflammation-inducing substance;
(b) a gene associated with light damage to the retina, but excluding the gene classified as (a);
(c) a gene associated with retinal inflammation caused by an inflammation-inducing substance, but excluding the gene classified as (a);
(d) a gene associated with both light damage to the retinal pigment epithelial cells and choroid and inflammation caused by an inflammation-inducing substance;
(e) a gene associated with light damage to the retinal pigment epithelial cells and choroid, but excluding the gene classified as (d);
(f) a gene associated with inflammation of the retinal pigment epithelial cells and choroid caused by an inflammation-inducing substance, but excluding the gene classified as (d).

2. A method for evaluating inhibitory or restorative functions of a test substance for an eye disease, comprising:
measuring the expression variation of a gene with a microarray, wherein the gene is classified as at least one selected from the group consisting of:
(a) a gene associated with both light damage to the retina and retinal inflammation caused by an inflammation-inducing substance;
(b) a gene associated with light damage to the retina, but excluding the gene classified as (a);
(c) a gene associated with retinal inflammation caused by an inflammation-inducing substance, but excluding the gene classified as (a);
(d) a gene associated with both light damage to the retinal pigment epithelial cells and choroid and inflammation caused by an inflammation-inducing substance;
(e) a gene associated with light damage to the retinal pigment epithelial cells and choroid, but excluding the gene classified as (d);
(f) a gene associated with inflammation of the retinal pigment epithelial cells and choroid caused by an inflammation-inducing substance, but excluding the gene classified as (d).

3. The method according to claim 1, wherein: the gene classified as (a) is at least one cluster selected from the group consisting of At1r, Jak3, Ccnd3, H2-K1, C1ql1, Id3, Tgfb2, Ckb, Gnat1, Efna5, Crx, Rom1, Arr3, Gsk3a, Adipor1, Hspa1b, Guk1, Abca4, egln3, Gngt2, Clip1, Prnp, Sparc, Elovl4, Gsk3b, Itgav, Vegfa, Vegfb, Vegfc, Vegfd, Sdc2, and Trip1;
the gene classified as (b) is at least one cluster selected from the group consisting of At2r, Pig7, Pgf, Rxrg, Col7a1, Casp9, Pecam1, Rpe65, Cckbr, Cd59a, Opn1mw, Grm6, Pkia, Darc, Apbb1, Prom1, Adam9, Cyb5r1, Gpr143, Atp6ap2, Nr2e3, Pde6a, Nr1, Cnga1, Hif1a, Gnat2, and Mmp2;
the gene classified as (c) is at least one cluster selected from the group consisting of Cxcl1, Il6, Selenbp2, Nfkb1, Cldn5, Sox9, Cp, Grm2, Pax6, Prkca, Mark2, Ppara, Gem, Opn1sw, Robo4, Rho, Glut1, and Pex1;
the gene classified as (d) is at least one cluster selected from the group consisting of cxcr4, At2r, Vcam1, Mef2c, Pkia, Scd1, Loxl1, H2-K1, Pxmp3, Erap1, Pgf, Tgfb3, Pdpn, Gsk3a, Fgf7, Ccl2, Cntf, Col8a2, Pecam1, Cd59a, Rxrg, C1s, Ccl7, Osbpl1a, Glut1, Selenbp2, Serpinf1, Gpnmb, Hspa2, Nes, Stat1, Egf, C1qb, Mmp14, Timp2, Lmo1, Lgals3, Mmp8, Flt1, Cldn5, Mmp2, Vegfa, Vegfb, Vegfc, Vegfd, Hspa1b, Kdr, and Stat6;
the gene classified as (e) is at least one cluster selected from the group consisting of Cfb, Mmp9, Calb2, Robo4, Gpr143, Cd44, Pig7, Il1b, C3, Gem, Cd55, Cebpd, Stat3, and Ccnd3; and
the gene classified as (f) is at least one cluster selected from the group consisting of Cxcl1, Timp1, Cxcl2, Il6, Igf1, Icam1, Lipc, At1r, Spp1, Ctss, C1qc, Nfkb1, Grem2, Abca4, Apbb1, Chrna7, Cyb5r1, Pdgfb, Isgf3g, Nos3, and Clip1.

4. The method according to claim 1, wherein the condition of an eye disease is evaluated using the determination formula:
M=(Xli×η1/β1+ . . . +Xki×ηk/(βk)(/η1+ . . . + [symbol] k) wherein:
when M1 max+σₘ₁<M2 min is satisfied, the condition of an evaluation sample cluster is the same as that in each disease model; and
“M” represents the Mahalanobis distance to show the distance from the base space, “Xni (n=1˜k)” represents a gene expression level or gene expression ratio, “ηn (n=1˜k)” represents a signal-to-noise ratio (S/N) in each sample, “βn (n=1˜k)” represents the sensitivity of each sample, “M1” represents a normal sample cluster or a control sample cluster, “M1 max” indicates the maximum value of “M1”, “σ m1” represents the standard deviation from “M1”, “M2” represents an evaluation sample cluster, and “M2 min” indicates the minimum value of “M2”.

5. A microarray configured to evaluate the condition of an eye disease, wherein the microarray is mounted with a nucleic acid or a part thereof, the nucleic acid being selected from the group consisting of:
(i) a nucleic acid comprising a gene selected from the group consisting of:
(a) a gene associated with both light damage to the retina and retinal inflammation caused by an inflammation-inducing substance;
(b) a gene associated with light damage to the retina, but excluding the gene classified as (a);
(c) a gene associated with retinal inflammation caused by an inflammation-inducing substance, but excluding the gene classified as (a);
(d) a gene associated with both light damage to the retinal pigment epithelial cells and choroid and inflammation caused by an inflammation-inducing substance;
(e) a gene associated with light damage to the retinal pigment epithelial cells and choroid, but excluding the gene classified as (d);
(f) a gene associated with inflammation of the retinal pigment epithelial cells and choroid caused by an inflammation-inducing substance, but excluding the gene classified as (d).
(ii) a nucleic acid comprising a base sequence complementary to the base sequence of the nucleic acid in (i); and
(iii) a nucleic acid capable of hybridizing under stringent conditions with a nucleic acid comprising a base sequence complementary to the base sequence of the nucleic acid in (i) or (ii).

6. The microarray according to claim 5, wherein:
the gene classified as (a) is at least one cluster selected from the group consisting of At1r, Jak3, Ccnd3, H2-K1, C1ql1, Id3, Tgfb2, Ckb, Gnat1, Efna5, Crx, Rom1, Arr3, Gsk3a, Adipor1, Hspa1b, Guk1, Abca4, egln3, Gngt2, Clip1, Prnp, Sparc, Elovl4, Gsk3b, Itgav, Vegfa, Vegfb, Vegfc, Vegfd, Sdc2, and Trip1;
the gene classified as (b) is at least one cluster selected from the group consisting of At2r, Pig7, Pgf, Rxrg, Col7a1, Casp9, Pecam1, Rpe65, Cckbr, Cd59a, Opn1mw, Grm6, Pkia, Darc, Apbb1, Prom1, Adam9, Cyb5r1, Gpr143, Atp6ap2, Nr2e3, Pde6a, Nr1, Cnga1, Hif1a, Gnat2, and Mmp2;
the gene classified as (c) is at least one cluster selected from the group consisting of Cxcl1, Il6, Selenbp2, Nfkb1, Cldn5, Sox9, Cp, Grm2, Pax6, Prkca, Mark2, Ppara, Gem, Opn1sw, Robo4, Rho, Glut1, and Pex1;
the gene classified as (d) is at least one cluster selected from the group consisting of cxcr4, At2r, Vcam1, Mef2c, Pkia, Scd1, Loxl1, H2-K1, Pxmp3, Erap1, Pgf, Tgfb3, Pdpn, Gsk3a, Fgf7, Ccl2, Cntf, Col8a2, Pecam1, Cd59a, Rxrg, C1s, Ccl7, Osbpl1a, Glut1, Selenbp2, Serpinf1, Gpnmb, Hspa2, Nes, Stat1, Egf, C1qb, Mmp14, Timp2, Lmo1, Lgals3, Mmp8, Flt1, Cldn5, Mmp2, Vegfa, Vegfb, Vegfc, Vegfd, Hspa1b, Kdr, and Stat6;
the gene classified as (e) is at least one cluster selected from the group consisting of Cfb, Mmp9, Calb2, Robo4, Gpr143, Cd44, Pig7, Il1b, C3, Gem, Cd55, Cebpd, Stat3, and Ccnd3; and
the gene classified as (f) is at least one cluster selected from the group consisting of Cxcl1, Timp1, Cxcl2, Il6, Igf1, Icam1, Lipc, At1r, Spp1, Ctss, C1qc, Nfkb1, Grem2, Abca4, Apbb1, Chrna7, Cyb5r1, Pdgfb, Isgf3g, Nos3, and Clip1.

7. A microarray configured to evaluate the condition of an eye disease, comprising a probe equipped to detect at least one gene selected from the group consisting of Hspa1b, Gsk3a and H2-K1.

8. The method according to claim 2, wherein:
the gene classified as (a) is at least one cluster selected from the group consisting of At1r, Jak3, Ccnd3, H2-K1, C1ql1, Id3, Tgfb2, Ckb, Gnat1, Efna5, Crx, Rom1, Arr3, Gsk3a, Adipor1, Hspa1b, Guk1, Abca4, egln3, Gngt2, Clip1, Prnp, Sparc, Elovl4, Gsk3b, Itgav, Vegfa, Vegfb, Vegfc, Vegfd, Sdc2, and Trip1;
the gene classified as (b) is at least one cluster selected from the group consisting of At2r, Pig7, Pgf, Rxrg, Col7a1, Casp9, Pecam1, Rpe65, Cckbr, Cd59a, Opn1mw, Grm6, Pkia, Darc, Apbb1, Prom1, Adam9, Cyb5r1, Gpr143, Atp6ap2, Nr2e3, Pde6a, Nr1, Cnga1, Hif1a, Gnat2, and Mmp2;
the gene classified as (c) is at least one cluster selected from the group consisting of Cxcl1, Il6, Selenbp2, Nfkb1, Cldn5, Sox9, Cp, Grm2, Pax6, Prkca, Mark2, Ppara, Gem, Opn1sw, Robo4, Rho, Glut1, and Pex1;
the gene classified as (d) is at least one cluster selected from the group consisting of cxcr4, At2r, Vcam1, Mef2c, Pkia, Scd1, Loxl1, H2-K1, Pxmp3, Erap1, Pgf, Tgfb3, Pdpn, Gsk3a, Fgf7, Ccl2, Cntf, Col8a2, Pecam1, Cd59a, Rxrg, C1s, Ccl7, Osbpl1a, Glut1, Selenbp2, Serpinf1, Gpnmb, Hspa2, Nes, Stat1, Egf, C1qb, Mmp14, Timp2, Lmo1, Lgals3, Mmp8, Flt1, Cldn5, Mmp2, Vegfa, Vegfb, Vegfc, Vegfd, Hspa1b, Kdr, and Stat6;
the gene classified as (e) is at least one cluster selected from the group consisting of Cfb, Mmp9, Calb2, Robo4, Gpr143, Cd44, Pig7, Il1b, C3, Gem, Cd55, Cebpd, Stat3, and Ccnd3; and
the gene classified as (f) is at least one cluster selected from the group consisting of Cxcl1, Timp1, Cxcl2, Il6, Igf1, Icam1, Lipc, At1r, Spp1, Ctss, C1qc, Nfkb1, Grem2, Abca4, Apbb1, Chrna7, Cyb5r1, Pdgfb, Isgf3g, Nos3, and Clip1.

9. The method according to claim 2, wherein the condition of an eye disease is evaluated using the determination formula:
M=(Xli×η1/β1+ . . . +Xki×ηk/(βk)(/η1+ . . . + [symbol] k)
wherein:
when M1 max+σₘ₁<M2 min is satisfied, the condition of an evaluation sample cluster is the same as that in each disease model; and
“M” represents the Mahalanobis distance to show the distance from the base space, “Xni (n=1˜k)” represents a gene expression level or gene expression ratio, “ηn (n=1˜k)” represents a signal-to-noise ratio (S/N) in each sample, “13n (n=1˜k)” represents the sensitivity of each sample, “M1” represents a normal sample cluster or a control sample cluster, “M1 max” indicates the maximum value of “M1”, “σ m1” represents the standard deviation from “M1”, “M2” represents an evaluation sample cluster, and “M2 min” indicates the minimum value of “M2”.

10. The method according to claim 3, wherein the condition of an eye disease is evaluated using the determination formula:
M=(Xli×η1/β1+ . . . +Xki×ηk/(βk)(/η1+ . . . + [symbol] k)
wherein:
when M1 max+σₘ₁<M2 min is satisfied, the condition of an evaluation sample cluster is the same as that in each disease model; and
“M” represents the Mahalanobis distance to show the distance from the base space, “Xni (n=1˜k)” represents a gene expression level or gene expression ratio, “ηn (n=1˜k)” represents a signal-to-noise ratio (S/N) in each sample, “βn (n=1˜k)” represents the sensitivity of each sample, “M1” represents a normal sample cluster or a control sample cluster, “M1 max” indicates the maximum value of “M1”, “σm1” represents the standard deviation from “M1”, “M2” represents an evaluation sample cluster, and “M2 min” indicates the minimum value of “M2”.
